(19) United States
(12) Patent Application Publication
(10) Pub. No.: US 2005/0137191 A1
(43) Pub. Date: Jun. 23, 2005

(54) Nitrate esters and their use for mitigating cellular damage

(76) Inventors: Gregory R.J. Thatcher, Chicago, IL (US); Brian M. Bennett, Kingston (CA); James N. Reynolds, Kingston (CA); Roland J. Boegman, Kingston (CA); Khem Jhamandas, Kingston (CA)

Correspondence Address: CLARK & ELBING LLP, 101 FEDERAL STREET, BOSTON, MA, 02110, US

(21) Appl. No.: 10/943,264

(22) Filed: Sep. 17, 2004

Related U.S. Application Data
(63) Continuation-in-part of application No. 10/147,808, filed on May 20, 2002.
Application No. 10/147,808 is a division of application No. 09/267,379, filed on Mar. 15, 1999, now Pat. No. 6,310,052.
Application No. 09/267,379 is a continuation-in-part of application No. 08/867,856, filed on Jun. 3, 1997, now Pat. No. 5,883,122.
Application No. 08/867,856 is a continuation-in-part of application No. 08/658,145, filed on Jun. 4, 1996, now Pat. No. 5,807,847.
(63) Continuation-in-part of application No. 09/473,713, filed on Dec. 29, 1999.

(51) Int. Cl. A61K 31/537; A61K 31/455; A61K 31/381; C07D 265/30; C07D 339/02

(52) U.S. Cl. 514/232.2; 514/509; 514/355; 514/406; 514/464; 514/440; 514/365; 544/162; 546/315; 549/20; 549/39; 546/280.1; 558/484

(57) ABSTRACT

Nitrate esters and methods for mitigating neurodegeneration, affecting neuroprotection, affecting cognition enhancement, and/or preventing or mitigating tissue and/or cellular damage in a subject are described. Neurological or cognitive conditions, or damage mediated by free radicals are treated by administering to a subject an effective amount of a therapeutic compound comprising a nitrate ester, or a pharmaceutically acceptable salt thereof.

### CROSS REFERENCE TO RELATED APPLICATIONS

This application is a continuation-in-part of application Ser. No. 10/147,808, filed May 20, 2002, which is a division of application Ser. No. 09/267,379, filed Mar. 15, 1999 and issued on Oct. 30, 2001 as U.S. Pat. No. 6,310,052, which is in turn a continuation-in-part of application Ser. No. 08/867,856, filed Jun. 3, 1997 and issued Mar. 16, 1999 as U.S. Pat. No. 5,883,122, which is in turn a continuation-in-part of application Ser. No. 08/658,145, filed Jun. 4, 1996 and issued Sep. 15, 1998 as U.S. Pat. No. 5,807,847. This application also claims the benefit of application Ser. No. 09/473,713, filed Dec. 29, 1999. Each of above applications is hereby incorporated by reference in its entirety.

### BACKGROUND OF THE INVENTION

This invention relates to nitrate esters and use thereof in mitigating cellular damage. Particularly, this invention relates to selected organic nitrates, most particularly those bearing a sulfur or phosphorus atom β or γ to the nitrate group, having therapeutic utility as agents that protect tissues from oxidative injury.

The nitrate ester glyceryl trinitrate (GTN) or nitroglycerin, has been used as a vasodilator in the treatment of angina pectoris for over a hundred years, and the dominant contemporary belief is that GTN exerts its therapeutic effect through in vivo release of nitric oxide (NO). Other organic nitrates, such as isosorbide dinitrate, have also been identified as effective and clinically important vasodilators. NO itself has been identified as Endothelium Derived Relaxing Factor (EDRF) and several classes of compounds, for example nitrosothiols, in addition to organic nitrates, have been proposed as NO donors or NO prodrugs. Endogenous stimulation or exogenous administration of NO have been shown to inhibit production of reactive oxygen species (ROS) and expression of oxidant-mediated molecular or tissue injury. Well-known examples of these classes of compounds and one nitrate, GTN itself, have been suggested to demonstrate neurotoxic or neuroprotective effects by dint of interactions with the redox modulatory site of the N-methyl-D-aspartate (NMDA) excitatory amino acid receptor. Thus GTN is firstly a potent vasodilator and secondly possesses potential neuroprotective properties. In addition, GTN has been found to suppress renal oxidant damage caused by potassium bromate (Rahman et al., Redox Rep. 4: 263-9, 1999). Several attempts have been made to increase the efficacy or potency of alternative organic nitrates as vasodilators relative to GTN, for example, by incorporation of propanolamine or cysteine functionalities. However, no attempt has been made to separately regulate the vasodilatory and cytoprotective effects of GTN. Indeed, postural hypotension and weakness are signs of cerebral ischemia, and are adverse effects associated with the vasodilatory effects of GTN. Observed in treatment, these effects are highly contraindicative of GTN itself, and by extrapolation GTN derivatives (1,2,3-trinitratopropane derivatives), as clinically useful protective therapeutic agents.

### SUMMARY OF THE INVENTION

In as much as the potent vasodilatory effects of organic nitrates may prove (a) deleterious to, or alternatively (b) synergistic with the protective effects of GTN, it is postulated herein that regulation of these two effects is required for development of new and useful protective therapeutic agents. Further, it is postulated that such regulation may be achieved through use of an appropriate organic nitrate, such as, for example, nitrate esters incorporating sulfur-containing or phosphorus-containing functionalities into the structure of the nitrate esters.

Accordingly, a first aspect the invention features nitrate esters having the formula: 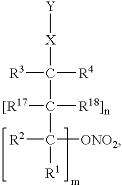
or a pharmaceutically acceptable salt thereof,
wherein:
each of m and n is, independently, an integer from 0 to 10;
each of R³, R⁴, R¹⁷ is, independently, hydrogen, a nitrate group, or A;
R¹ is hydrogen or A;
where A is selected from: a substituted or unsubstituted aliphatic group having from 1 to 24 carbon atoms in the chain, which optionally contains 1 to 4 O, S, NR⁶, and/or unsaturations in the chain, optionally bearing from 1 to 4 hydroxy, nitrate, amino, aryl, or heterocyclic groups; an unsubstituted or substituted cyclic aliphatic moiety having from 3 to 7 carbon atoms in the aliphatic ring, which optionally contains 1 to 2 O, S, NR⁶, and/or unsaturations in the ring, optionally bearing from 1 to 4 hydroxy, nitrate, amino, aryl, or heterocyclic groups; an unsubstituted or substituted aliphatic moiety comprising a linkage from 0 to 5 carbon atoms between R¹ and R³ and/or between R¹⁷ and R⁴, which optionally contains 1 to 2 O, S, NR⁶, and/or unsaturations in the linkage, optionally bearing from 1 to 4 hydroxy, nitrate, amino, aryl, or heterocyclic groups; a substituted or unsubstituted aliphatic group having from 1 to 24 carbon atoms in the chain, containing linkages selected from C═O, C═S, and C═NOH, which optionally contains 1 to 4 O, S, NR⁶, and/or unsaturations in the chain, optionally bearing from 1 to 4 hydroxy, nitrate, amino, aryl, or heterocyclic groups; a substituted or unsubstituted aryl group; a heterocyclic group; an amino group selected from alkylamino, dialkylamino, cyclic amino, cyclic diamino, cyclic triamino, arylamino, diarylamino, and alkyarylamino; a hydroxy group; an alkoxy group; and a substituted or unsubstituted aryloxy group;
each of R², R⁵, R¹⁸ is, independently, hydrogen, A, or X-Y;
where X is F, Br, Cl, NO₂ CH₂, CF₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂H, PO₂M, P(O)(OR¹⁴), P(O)(R¹³), SO, SO₂, C(O)(SR¹³), SR⁵, SSR⁷, or SSR⁵;
Y is F, Br, Cl, CH₃, CF₂H, CF₃, OH, NH₂, NHR⁶, NR⁶R⁷, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O)R¹², C(O)(OR¹³), C(O)(SR¹³), SR⁵, SSR⁷, or SSR⁵, or does not exist;
each of R⁶, R⁷, R⁸, R⁹, R¹¹, R¹², R¹³, R¹⁴, R¹⁵, R¹⁶ is, independently, an alkyl or acyl group containing 1-24 carbon atoms, which may contain 1-4 ONO₂ substituents;
a C₁-C₆ connection to R¹-R⁴ in a cyclic derivative; a hydrogen, a nitrate group, or A; and
M is H, Na⁺, K⁺, NH₄⁺, or N⁺HₖR¹¹₍₄₋ₖ₎ where k is 0 to 3, or other pharmaceutically acceptable counterion;
with the proviso that:
when m=0; n=1;
each of R¹⁸ and R³ is, independently, H, a nitrate group, or a C₁-C₄ alkyl chain, which may include one O linking R¹⁸ and R³ to form a pentosyl, hexosyl, cyclopentyl, or cyclohexyl ring, which ring optionally bears a hydroxyl substituent;
each of R¹⁷ and R⁴ is, independently H, a nitrate group, a C₁-C₄ alkyl, optionally bearing 1 to 3 nitrate groups, or an acyl group (—C(O)R⁵);
each of R⁵, R⁶, R⁸, R⁹, R¹², R¹³, R¹⁴, R¹⁵, R¹⁶ is, independently, an alkyl group containing 1 to 12 carbon atoms, which may contain 1 to 4 ONO₂ substituents; or a C₁ or C₂ connection to R¹⁸, R¹⁷, or R³ in a cyclic derivative;
each of R⁷ and R¹¹ is, independently, a C₁ to C₈ alkyl or acyl group;
M is H, Na⁺, K⁺, NH₄⁺, or N⁺HₖR¹¹₍₄₋ₖ₎ where k is 0 to 3; and
X is CH₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂M, P(O)(OR¹⁴), P(O)(R¹³), SO, SO₂, C(O)(SR¹³), or SSR⁴;
then Y is not CN, N₂H₂R¹³, N₂HR¹³R¹⁴, N₃, SCN, SC(═NH)N(R¹⁵)₂, SC(═NH)NHR¹⁵, SC(O)N(R¹⁵)₂, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R¹¹, C(O)R¹², C(O)(SR¹³), SR⁴, SR⁵, or SSR⁵, or Y does not exist.

In accordance with the present invention, one skilled in the recognizes that one of m, n, or p must be equal to at least one.

In one embodiment, the compound of the invention is: 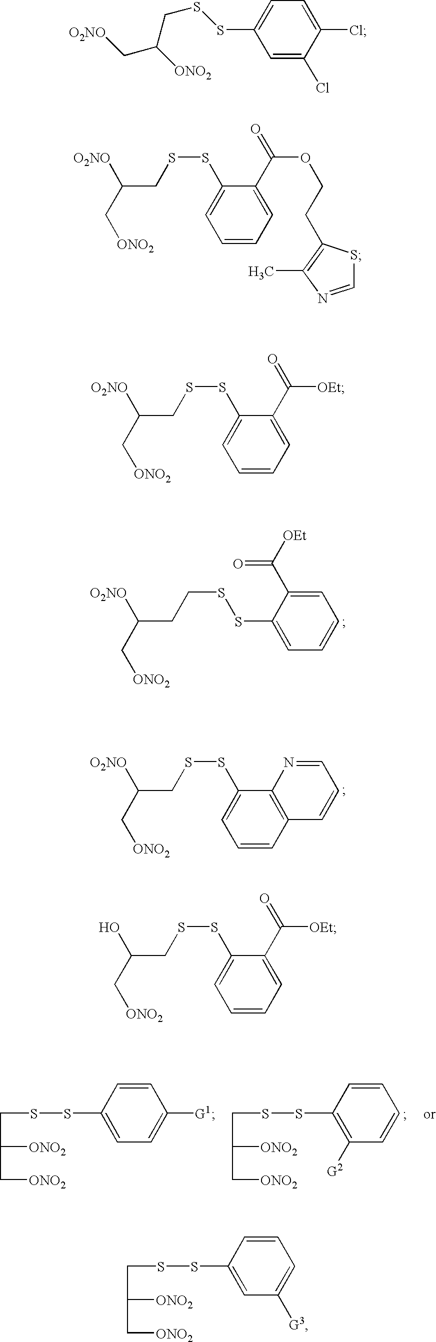
wherein G¹ is Me, OMe, Cl, NO₂, Br, or H; G² is CO₂Et, CO₂H, CO₂Me, CONH₂, or CO(CH₂)₂NEt₂; and G³ is Cl, OMe, or CONH₂.

In another embodiment, the compound is:

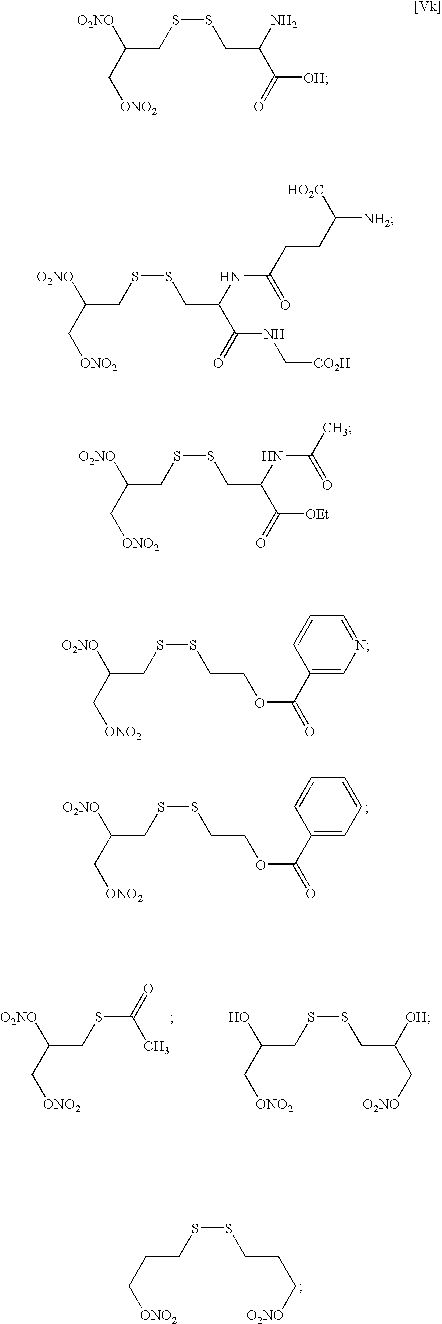

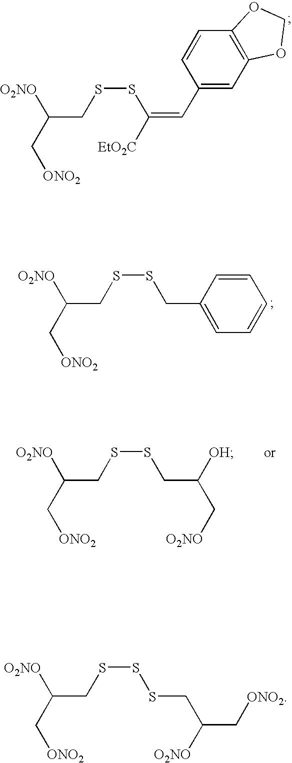

In another embodiment, the compound is: 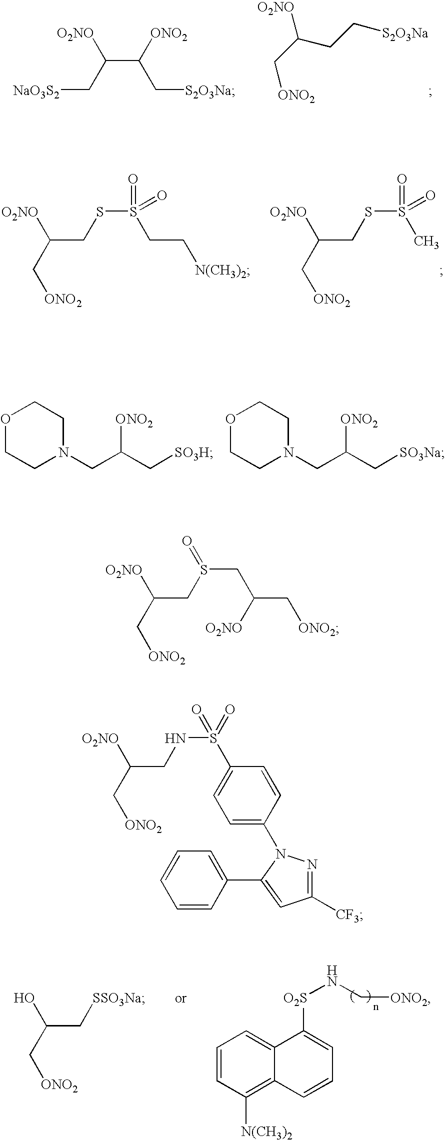
wherein n is 2 or 3.

In another embodiment, the compound is:

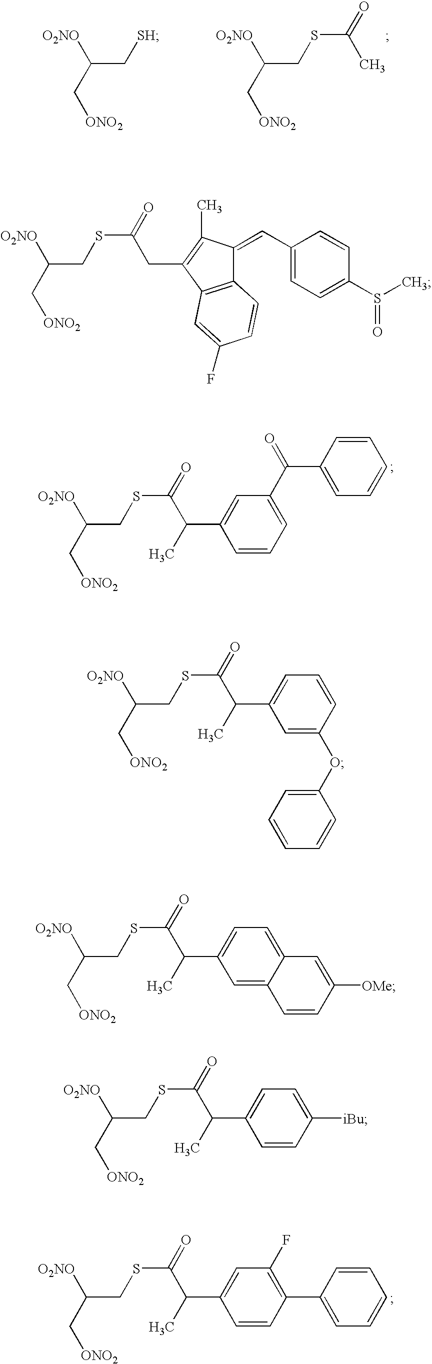

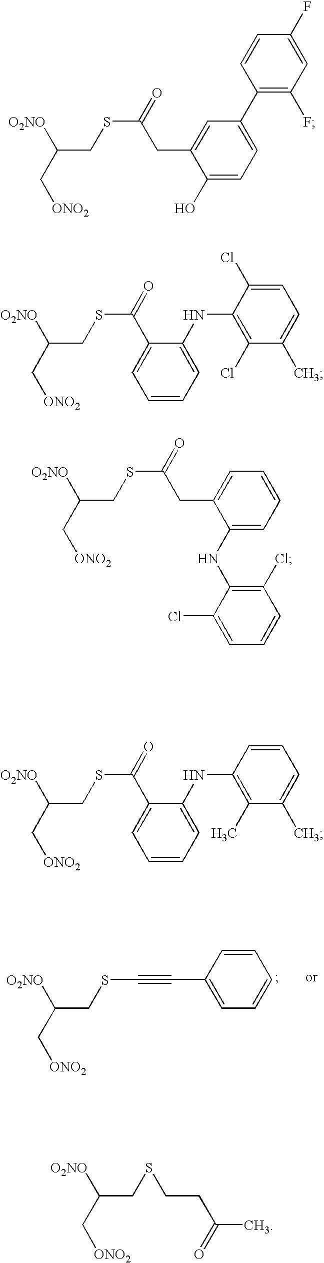

In another embodiment, the compound is:

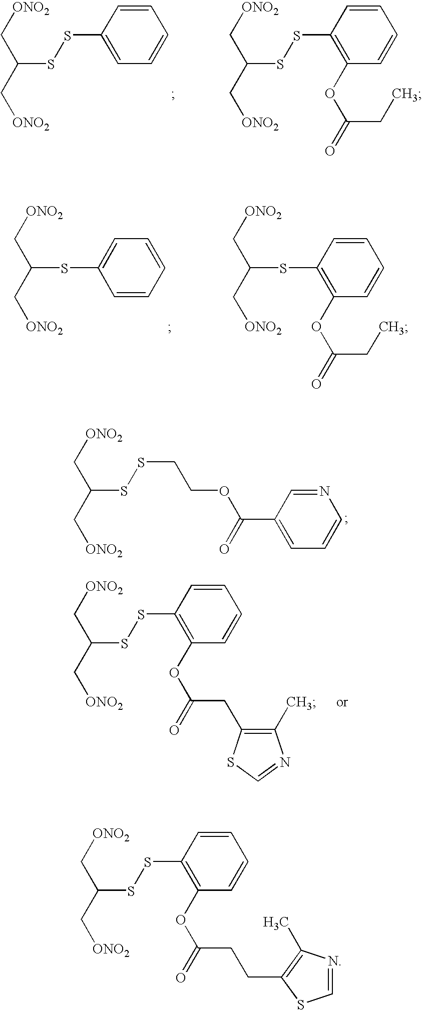

In yet another embodiment, the compound is:

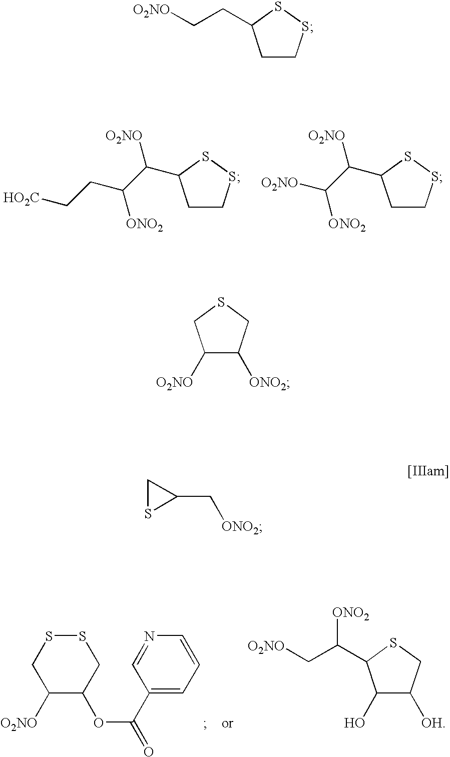

In a second aspect, the invention features a compound having the formula: 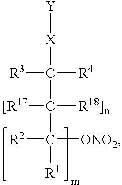
or a pharmaceutically acceptable salt thereof, containing from 1 to 3 nitrate groups and an S atom in proximity to a nitrate group,
wherein
each of m and n is, independently, an integer from 0 to 10;
R¹ is a hydrogen or A;
each of R², R⁵, and R¹⁸ is, independently, hydrogen or A;
each of R³, R⁴, and R¹⁷, is independently, a hydrogen, a nitrate group, or A;
each of R⁶, R⁷, R⁸, R⁹, R¹¹, R¹², R¹³, R¹⁴, R¹⁵, and R¹⁶ is, independently, A, a hydrogen, a nitrate group, or a C₁-C₂₄ alkyl or acyl group, optionally containing 1-4 ONO₂ substituents or a C₁-C₆ linkage to R¹, R², R³, or R⁴ in cyclic derivatives;
each of R⁷ and R¹¹ is, independently, a substituted or unsubstituted C₁-C₈ alkyl or acyl group;
A is selected from:
a C₁-C₂₄ alkyl group, which optionally contains 1 to 4 O, S, NR⁶, and/or unsaturations in the chain, optionally bearing from 1 to 4 hydroxy, Cl, F, amino, unsubstituted or substituted aryl, unsubstituted or substituted heteroaryl, or unsubstituted or substituted heterocyclic groups, or 1-2 nitrate groups;
a C₃-C₂₄ alkyl group, containing 1-5 C═O, C═S, or C═NOR⁷ linkages, which optionally contains 1 to 4 O, S, NR⁶, and/or unsaturations in the carbon chain, optionally bearing from 1 to 4 hydroxy, nitrate, Cl, F, amino, unsubstituted or substituted aryl, unsubstituted or substituted heteroaryl, or unsubstituted or substituted heterocyclic groups;
a C₃-C₇ linkage to any of R¹, R², R⁴, R⁴, or R¹⁷, forming an aliphatic ring, which optionally contains 1 to 2 O, S, NR⁶, and/or unsaturations in the linkage, optionally bearing from 1 to 6 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
a C₀-C₅ linkage to or between any of R¹, R³, R⁴, or R¹⁷, which optionally contains 1 to 2 O, S, NR⁶, and/or unsaturations in the linkage, bearing two or more substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
an unsubstituted C₀-C₅ linkage to or between any of R¹, R³, and R⁴, which optionally contains 1 to 2 non-adjacent O, S, NR⁶, and/or unsaturations in the linkage;
a C₁-C₅ linkage to or between any of R¹, R³, R⁴, and R¹⁷ containing 1 to 2 C═O, C═S, or C═NOR⁷ linkages, which optionally contains O, S, NR⁶, and/or unsaturations in the linkage, optionally bearing from 1 to 4 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
a substituted or unsubstituted aryl group;
a substituted or unsubstituted heteroaryl group;
a substituted or unsubstituted heterocyclic group;
an amino, cyclic amino, diamino, triamino, alkylamino, dialkylamino, arylamino, diarylamino, or alkylarylamino group;
a hydroxy group;
an alkoxy group; and
a substituted or unsubstituted aryloxy group;
X is F, Br, NO₂, CH₂ CF₂, O, NH, NMe, NHOH, N₂H₃, N₂H₂R¹³, N₂HR¹³R¹⁴, N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)R⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷, or does not exist;
Y is F, Br, CH₃, CF₂H, CF₃, OH, NH₂, NHR⁶, NR⁶R⁷, NHOH, N₂H₃, N₂H₂R¹³, N₂HR¹³R¹⁴, N₃, SC(═NH)N(R¹⁵)₂, SC(═NH)NHR¹⁵, SC(O)N(R¹⁵)₂, SC(O)NHR¹⁵, SH, SR⁷, SO₂M, S(O)R⁸, S(O)R⁵, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R⁵, C(O)(OR¹³), C(O)(SR¹³), C(S)R⁵, C(S)R¹², C(S)OR¹², or does not exist; and
M is H, Na⁺, K⁺, NH₄⁺, N⁺HₖR¹¹₍₄₋ₖ₎ where k is 0-3, or other pharmaceutically acceptable counterion;
and with the proviso that,
when m=0 and n=1;
each of R¹⁸ and R³ is, independently, a hydrogen, a nitrate group, or a C₁-C₄ alkyl chain, which may include one O, linking R¹⁸ and R³ together to form a pentosyl, a hexosyl, a cyclopentyl, or a cyclohexyl ring, said ring optionally bearing from 1-4 hydroxyl substituents;
each of R¹⁷ and R⁴ is, independently, a hydrogen, a nitrate group, a C₁-C₄ alkyl group, optionally bearing from 1-3 nitrate groups, or an acyl group (—C(O)R⁵);
each of R⁵, R⁶, R⁸, R⁹, R¹², R¹³, R¹⁴, R¹⁵, and R¹⁶ is, independently, a C₁-C₁₂ alkyl group, optionally bearing from 1-4 ONO₂ substituents, or a C₁-C₂ alkyl linkage to R¹⁸, R¹⁷, or R³;
X is F, Br, NO₂, CH₂ CF₂, O, NH, NMe, NHOH, N₂H₃, N₂H₂R¹³, N₂HR¹³R¹⁴, N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)R⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷; and
Y is not CN, N₂H₂R¹³, N₂HR¹³R¹⁴, N₃, SCN, SC(═NH)N(R¹⁵)₂, SC(═NH)NHR¹⁵, SC(O)N(R¹⁵)₂, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R⁵C(O)R¹², C(O)(SR¹³), SR⁴, SR⁵, or SSR⁵, or Y does not exist.

In one embodiment of the second aspect, the compound is:

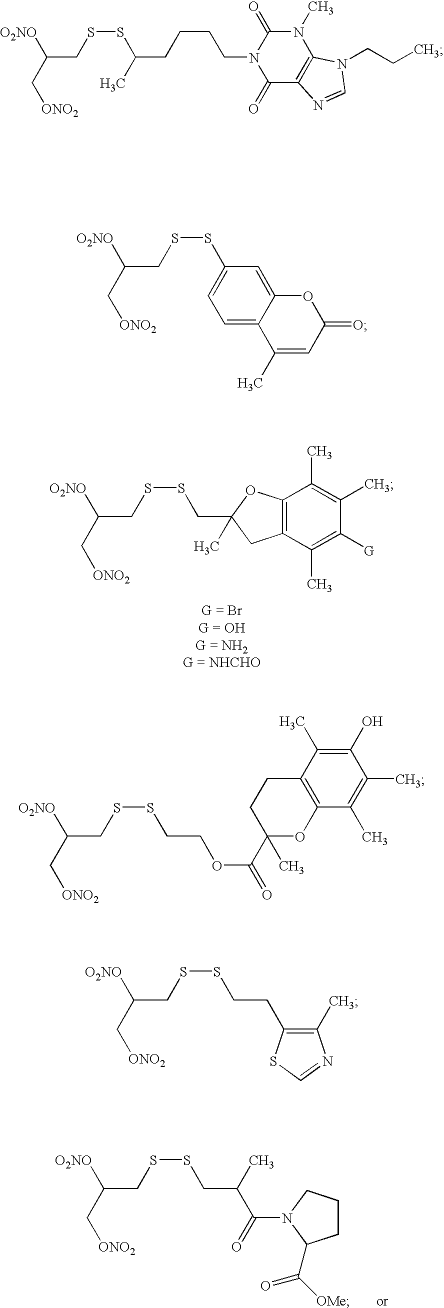

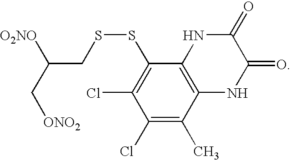

In another embodiment, the compound is:

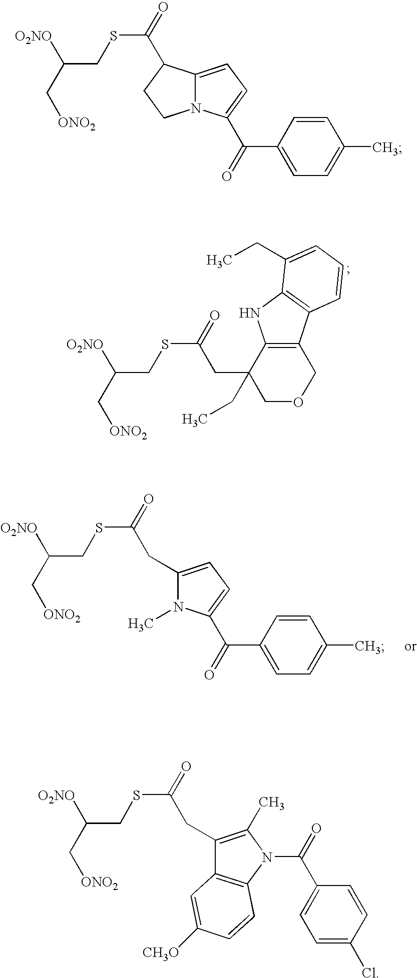

In another embodiment, the compound is:

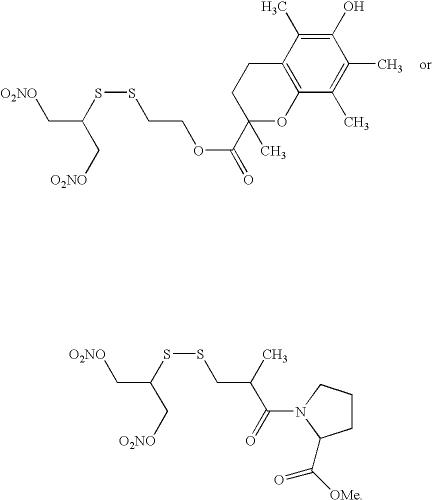

In another embodiment the compound is:

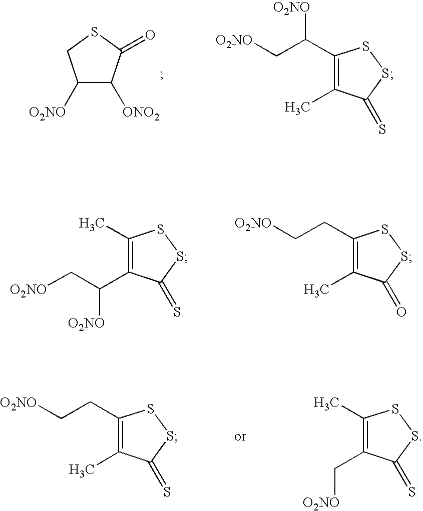

In another embodiment, the compound is:

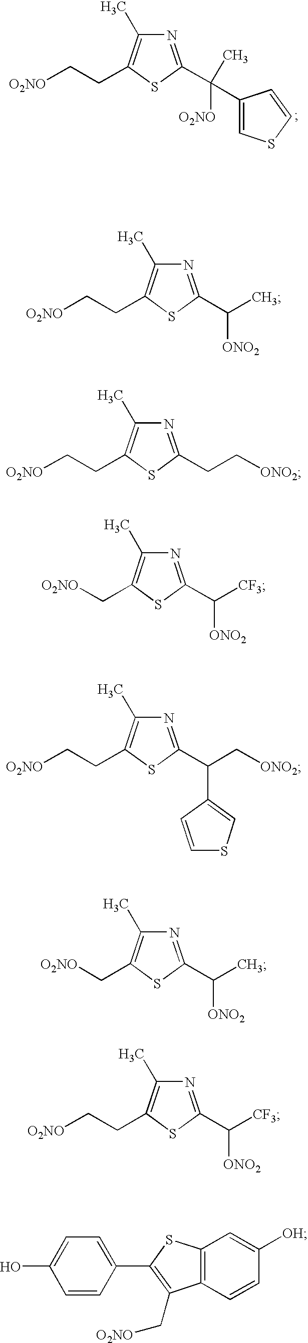

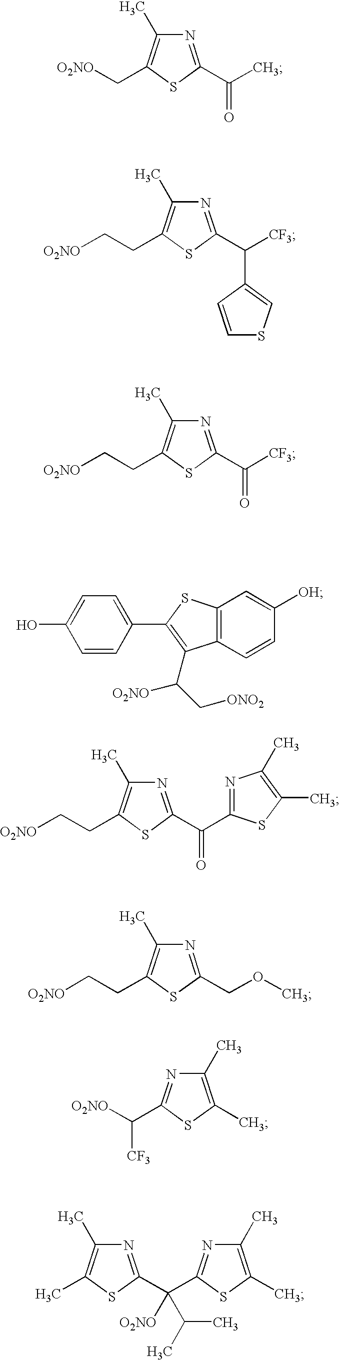

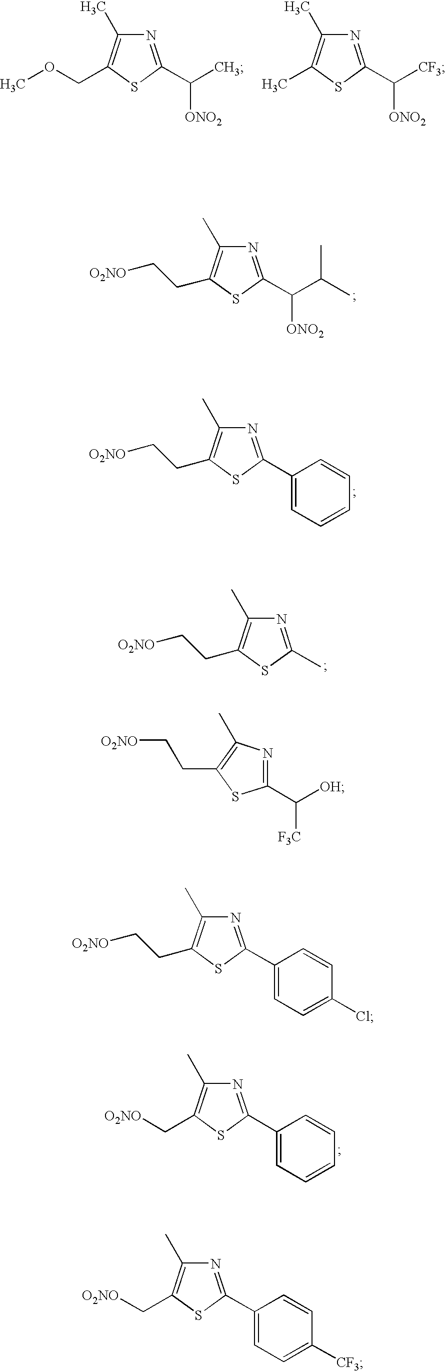

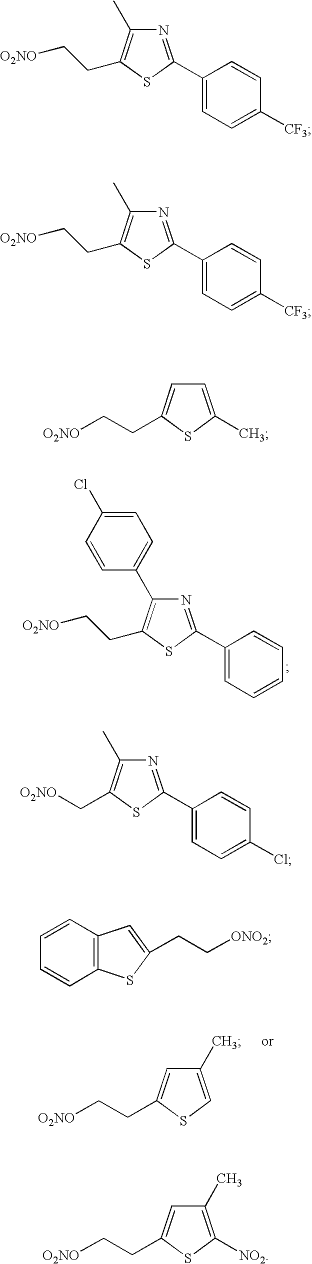

In another embodiment, the compound is:

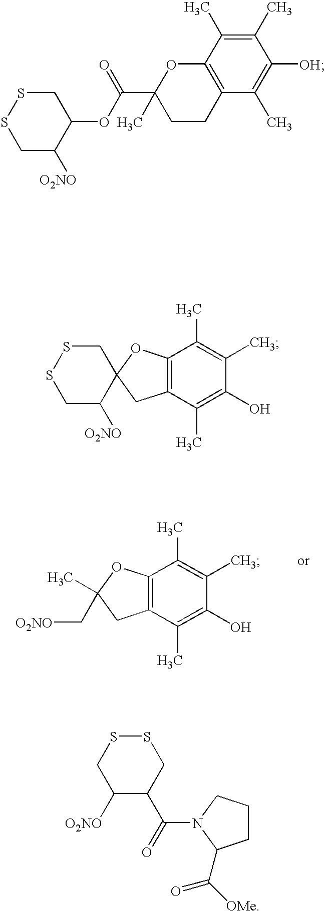

In yet another embodiment of the second aspect, the compound is:

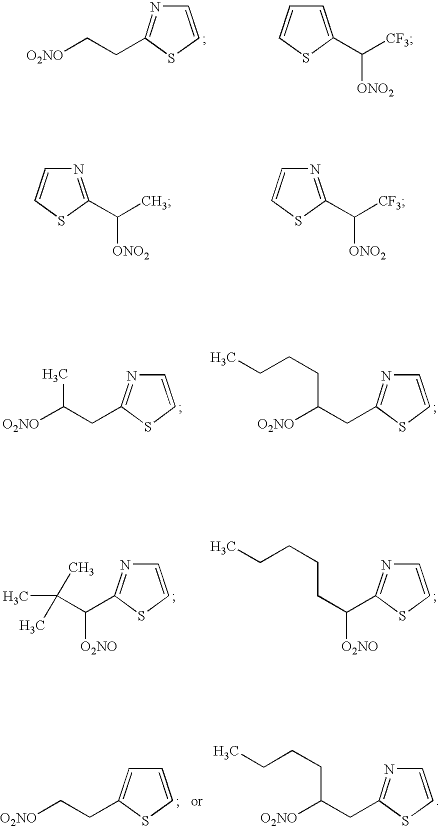

In a third aspect, the invention features a compound having the formula:

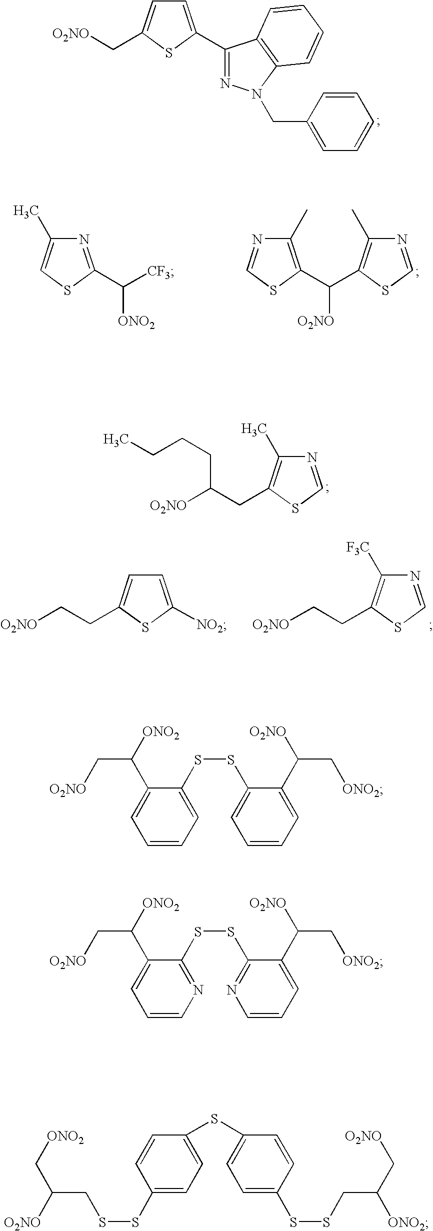

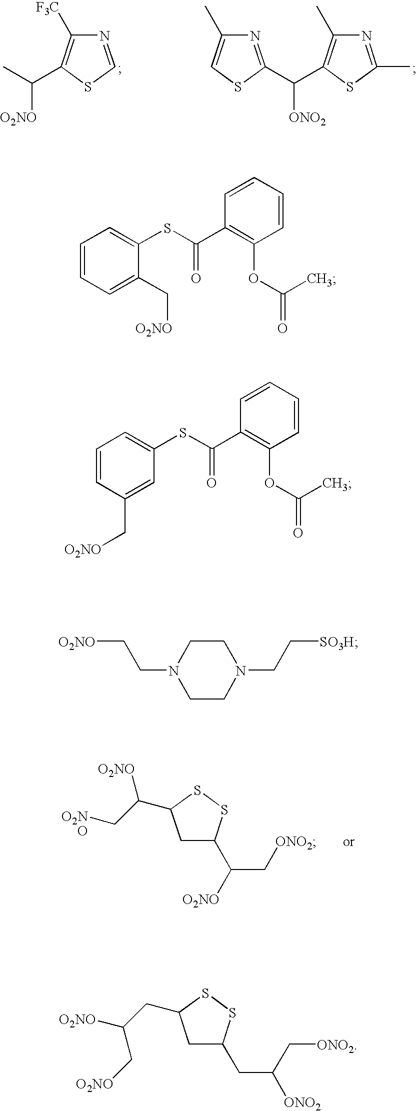

In a fourth aspect, the invention features a composition that includes a pharmaceutically acceptable carrier and a compound having the general formula: 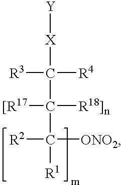
or a pharmaceutically acceptable salt thereof, containing 1 to 3 nitrate groups and an S atom in proximity to a nitrate group,
wherein
each of m and n is, independently, an integer from 0 to 10;
R¹ is a hydrogen or A;
each of R², R⁵, and R¹⁸ is, independently, hydrogen or A;
each of R³, R⁴, and R¹⁷, is independently, a hydrogen, a nitrate group, or A;
each of R⁶, R⁷, R⁸, R⁹, R¹¹, R¹², R¹³, R¹⁴, R¹⁵, and R¹⁶ is, independently, A, a hydrogen, a nitrate group, or a C₁-C₂₄ alkyl or acyl group, optionally containing 1-4 ONO₂ substituents or a C₁-C₆ linkage to R¹, R², R³, or R⁴ in cyclic derivatives;
each of R⁷ and R¹¹ is, independently, a substituted or unsubstituted C₁-C₈ alkyl or acyl group;
A is selected from:
a C₁-C₂₄ alkyl group, which optionally contains 1 to 4 O, S, NR⁶, and/or unsaturations in the chain, optionally bearing from 1 to 4 hydroxy, Cl, F, amino, unsubstituted or substituted aryl, unsubstituted or substituted heteroaryl, or unsubstituted or substituted heterocyclic groups, or 1-2 nitrate groups;
a C₃-C₂₄ alkyl group, containing 1-5 C═O, C═S, or C═NOR⁷ linkages, which optionally contains 1 to 4 O, S, NR⁶, and/or unsaturations in the carbon chain, optionally bearing from 1 to 4 hydroxy, nitrate, Cl, F, amino, unsubstituted or substituted aryl, unsubstituted or substituted heteroaryl, or unsubstituted or substituted heterocyclic groups;
a C₃-C₇ linkage to any of R¹, R², R³, R⁴, or R¹⁷, forming an aliphatic ring, which optionally contains 1 to 2 O, S, NR⁶, and/or unsaturations in the linkage, optionally bearing from 1 to 6 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
a C₀-C₅ linkage to or between any of R¹, R³, R⁴, or R¹⁷, which optionally contains 1 to 2 O, S, NR⁶, and/or unsaturations in the linkage, bearing two or more substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
an unsubstituted C₀-C₅ linkage to or between any of R¹, R³, and R⁴, which optionally contains 1 to 2 non-adjacent O, S, NR⁶, and/or unsaturations in the linkage;
a C₁-C₅ linkage to or between any of R¹, R³, R⁴, and R¹⁷ containing 1 to 2 C═O, C═S, or C═NOR⁷ linkages, which optionally contains O, S, NR⁶, and/or unsaturations in the linkage, optionally bearing from 1 to 4 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
a substituted or unsubstituted aryl group;
a substituted or unsubstituted heteroaryl group;
a substituted or unsubstituted heterocyclic group;
an amino, cyclic amino, diamino, triamino, alkylamino, dialkylamino, arylamino, diarylamino, or alkylarylamino group;
a hydroxy group;
an alkoxy group; and
a substituted or unsubstituted aryloxy group;
X is F, Br, NO₂, CH₂ CF₂, O, NH, NMe, NHOH, N₂H₃, N₂H₂R¹³, N₂HR¹³R¹⁴ , N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)R⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷, or does not exist;
Y is F, Br, CH₃, CF₂H, CF₃, OH, NH₂, NHR⁶, NR⁶R⁷, NHOH, N₂H₃, N₂H₂R¹³N₂HR¹³R¹⁴, N₃, SC(═NH)N(R¹⁵)₂, SC(═NH)NHR¹⁵, SC(O)N(R¹⁵)₂, SC(O)NHR¹⁵, SH, SR⁷, SO₂M, S(O)R⁸, S(O)R⁵, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R⁵, C(O)(OR¹³), C(O)(SR¹³), C(S)R⁵, C(S)R¹², C(S)OR¹², or does not exist; and
M is H, Na⁺, K⁺, NH₄⁺, N⁺HₖR¹¹₍₄₋ₖ₎ where k is 0-3, or other pharmaceutically acceptable counterion;
and with the proviso that,
when m=0 and n=1;
each of R¹⁸ and R³ is, independently, a hydrogen, a nitrate group, or a C₁-C₄ alkyl chain, which may include one O, linking R¹⁸ and R³ together to form a pentosyl, a hexosyl, a cyclopentyl, or a cyclohexyl ring, said ring optionally bearing from 1-4 hydroxyl substituents;
each of R¹⁷ and R⁴ is, independently, a hydrogen, a nitrate group, a C₁-C₄ alkyl group, optionally bearing from 1-3 nitrate groups, or an acyl group (—C(O)R⁵);
each of R⁵, R⁶, R⁸, R⁹, R¹², R¹³, R¹⁴, R¹⁵, and R¹⁶ is, independently, a C₁-C₁₂ alkyl group, optionally bearing from 1-4 ONO₂ substituents, or a C₁-C₂ alkyl linkage to R¹⁸, R¹⁷, or R³;
X is F, Br, NO₂, CH₂ CF₂, O, NH, NMe, NHOH, N₂H₃, N₂H₂R¹³, N₂HR¹³R¹⁴, N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)R⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷; and
Y is not CN, N₂H₂R¹³, N₂HR¹³R¹⁴, N₃, SCN, SC(═NH)N(R¹⁵⁾₂, SC(═NH)NHR¹⁵, SC(O)N(R¹⁵)₂, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R⁵, C(O)R¹², C(O)(SR¹³), SR⁴, SR⁵, or SSR⁵, or Y does not exist.

In a fifth aspect, the invention features a composition that includes a pharmaceutically acceptable carrier and one of the following compounds:

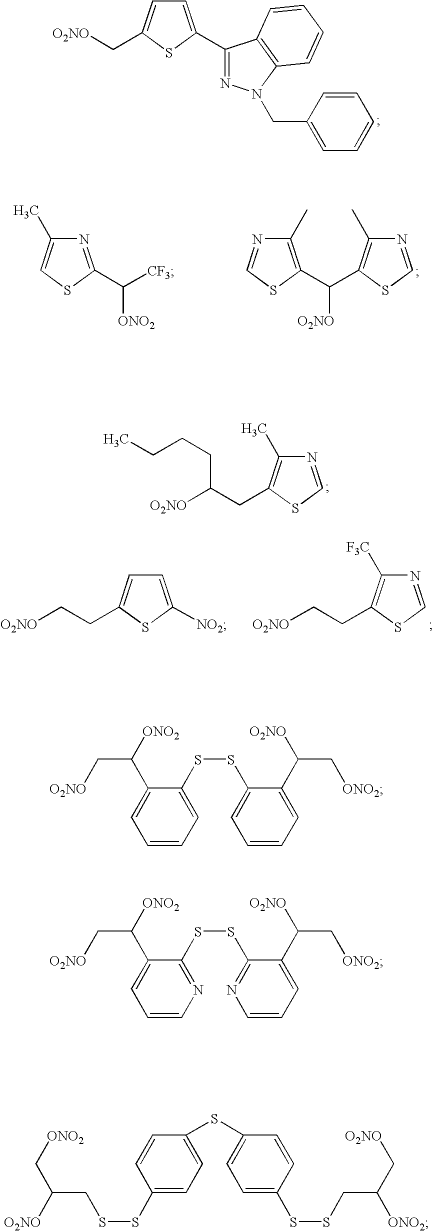

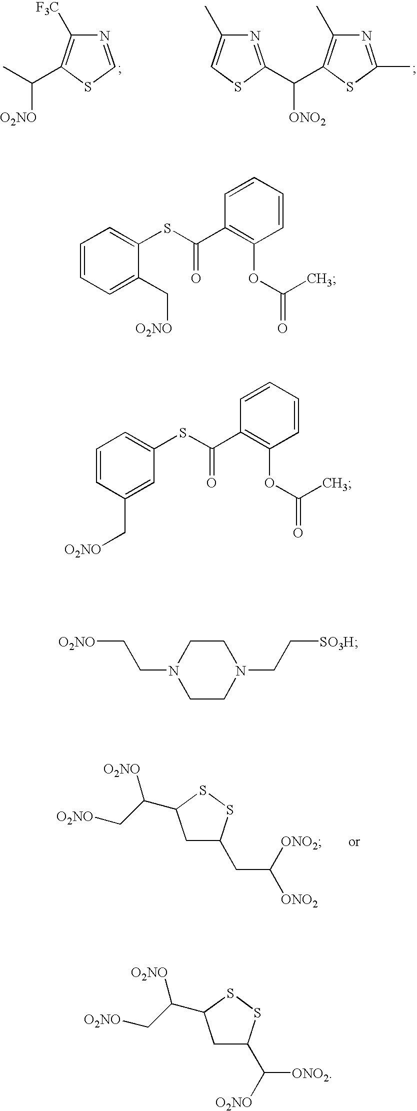

In a sixth aspect, the invention features a method for preventing or mitigating tissue and/or cellular damage in a subject by modulating intercellular and/or intracellular free radical concentration in the subject by administering to the subject an effective amount of a compound containing at least one aliphatic nitrate group and at least one sulfur atom in proximity to the nitrate group. By “sulfur atom in proximity” or “proximal functional group” is meant a sulfur atom or functional group that is connected through bonds in a β, γ, or δ relationship to a nitrate ester group (i.e., the atom connectivity is 1,2, or 1,3, or 1,4). The functional group may also be referred to as “proximally located” or “situated in proximity.” Proximal functional groups also include those groups that have a through-space intramolecular juxtaposition with a nitrate group that is within 3 Å.

In one embodiment, the tissue and/or cellular damage can be associated with aging, septic shock, ischemia/reperfusion injury, ulcers, gastritis, ulcerative colitis, Crohn's disease, diabetes, rheumatoid arthritis, asthma, cirrhosis of the liver, allograft rejection, encephalomyelitis, meningitis, pancreatitis, peritonitis, vasculitis, lymphocytic choriomeningitis, glomerulonephritis, uveitis, glaucoma, blepharitis, chalazion, allergic eye disease, corneal ulcer, keratitis, cataracts, age-related macular degeneration, optic neuritis, ileitis, hemorrhagic shock, anaphylactic shock, bacterial infection, viral infection, fungal infection, parasitic infection, hemodialysis, chronic fatigue syndrome, stroke, toxic shock syndrome, adult respiratory distress syndrome, cachexia, myocarditis, eczema, psoriasis, dermatitis, urticaria, cerebral ischemia, systemic lupus erythematosis, chronic neurodegenerative disease, priapism, cystic fibrosis, schizophrenia, depression, premenstrual syndrome, anxiety, addiction, migraine, gastrointestinal motility disorders, obesity, hyperphagia, hematologic cancers, myelofibrosis, graft-versus-host disease, CNS trauma, hepatitis, renal failure, chronic hepatitis C, drug-induced lung injury (e.g., paraquat), bacterial translocation, circulatory shock, traumatic shock, vascular aneurysm, metastatic cancer, or myocardial infarction.

In another embodiment, the tissue and/or cellular damage can associated with neurological diseases such as, for example, Parkinson's disease; Alzheimer's disease; Huntington's disease; multiple sclerosis; amylotrophic lateral sclerosis; AIDS-induced dementia; epilepsy; alcoholism; alcohol withdrawal; drug-induced seizures; viral/bacterial/fever-induced seizures; trauma to the head; hypoglycemia; hypoxia due to myocardial infarction; cerebral vascular occlusion; cerebral vascular hemorrhage; hemorrhage; or environmental excitotoxins of plant, animal, or marine origin.

In yet another embodiment, the tissue and/or cellular damage can be associated with cytokine therapy, wherein a nitrate ester of the invention is administered to the subject before, during, and/or after the administration of the therapeutic cytokine.

In another embodiment of the sixth aspect, the compound has the formula: 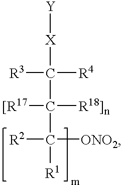
containing from 1 to 3 nitrate groups and an S atom in proximity to a nitrate group, wherein
m is an integer from 0 to 10;
n is an integer from 0 to 10;
each of R^{3,4,17} is, independently, hydrogen, a nitrate group, or A;
R¹ is hydrogen or A;
A is selected from:
a substituted or unsubstituted C₁-C₂₄ alkyl group, optionally containing 1 to 4 O, S, NR⁶, and/or unsaturations in the chain, optionally bearing from 1 to 4 hydroxy, nitrate, Cl, F, amino or unsubstituted or substituted aryl, or unsubstituted or substituted heterocyclic groups;
an unsubstituted or substituted cyclic moiety having from 3 to 7 carbon atoms in the ring, which optionally containing 1 to 2 O, S, NR⁶, and/or unsaturations in the ring, optionally bearing from 1 to 4 hydroxy, nitrate, Cl, F, amino or unsubstituted or substituted aryl, or unsubstituted or substituted heterocyclic groups;
an unsubstituted or substituted moiety constituting a linkage from 0 to 5 carbons, to or between any of R¹, R², R¹⁷ and R⁴, which optionally contains 1 to 4 O, S, NR⁶, and/or unsaturations in the linkage, and optionally bearing from 1; to 4 hydroxy, nitrate, Cl, F, amino or unsubstituted or substituted aryl, or unsubstituted or substituted heterocyclic groups;
a substituted or unsubstituted C₁-C₂₄ alkyl group, containing 1-4 linkages selected from C═O, C═S, and C═NOR, which optionally contains O, S, NR⁶, and/or unsaturations in the chain, optionally bearing from 1 to 4 hydroxy, nitrate, Cl, F, amino or unsubstituted or substituted aryl, or unsubstituted or substituted heterocyclic groups;
a substituted or unsubstituted aryl group;
a substituted or unsubstituted heterocyclic group;
an amino group selected from alkylamino, dialkylamino., cyclic amino, cyclic diamino, cyclic triamino, arylamino, diarylamino, and alkyarylamino;
a hydroxy group;
an alkoxy group; and
a substituted or unsubstituted aryloxy group;
R², R⁵, R¹⁸, are optionally hydrogen, A, or X-Y
X is F, Br, Cl, NO₂, CH₂, CF₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)R⁵, S(O)₂R⁵, S(O)OR⁸, S(O)₂OR⁹, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂H, PO₂M, P(O)(OR¹⁴), P(O)(R¹³), SO, SO₂, C(O)(SR¹³), SR⁵, SSR⁷ or SSR⁵, SS or does not exist;
Y is F, Br, Cl, CH₃, CF₂H, CF₃, OH, NH₂, NHR⁶, NR⁶R⁷, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)R⁵, S(O)₂R⁵, S(O)₂OR⁹, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R⁵, C(O)R¹², C(O)(OR¹³), C(O)(SR¹³), SR⁵, SSR⁷ or SSR⁵, C(S)R⁵, C(S)R¹², C(S)OR¹², or does not exist; each of R⁶, R⁷, R⁸, R⁹, R¹¹, R¹², R¹³, R¹⁴, R¹⁵, and R¹⁶ is, independently, a C₁-C₂₄ alkyl group, optionally containing 1-4 ONO₂ substituents, a C₁-C₂₄ acyl group, optionally containing 1-4 ONO₂ substituents, a C₁-C₆ ring-forming connection to any of R¹-R⁴, a hydrogen, a nitrate group, or A; and
M is H, Na⁺, K⁺, NH₄+, N+HₖR¹¹₍₄₋ₖ₎ where k is 0-3, or other pharmaceutically acceptable counterion;
and with the proviso that,
when m=0; n=1;
each of R¹⁸ and R³ is, individually, H, a nitrate group, or a C₁-C₄ alkyl group, which may include one O, linking R¹⁸ and R³ to form pentosyl, hexosyl, cyclopentyl, or cyclohexyl rings, which optionally bears hydroxyl substituents;
each of R¹⁷ and R⁴ is, individually, H, a nitrate group, a C₁-C₄ alkyl group, optionally bearing from 1-3 nitrate groups, or —C(O)R⁵;
each of R⁵, R⁶, R⁸, R⁹, R¹², R¹³, R¹⁴ , R¹⁵, R¹⁶ is, individually, a C₁-C₁₂ alkyl group, optionally containing 1-4 ONO₂ substituents or a C₁-C₂ ring-forming connections to R¹⁸, R¹⁷, or R³;
each of R⁷ and R¹¹ is, independently, a C₁-C₈ alkyl group or a C₁-C₈ acyl group;
M is H, Na⁺, K⁺, NH₄+, N+HₖR¹¹₍₄₋ₖ₎ where k is 0-3;
X is CH₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂M, P(O)(OR¹⁴) P(O)(R¹³), SO, SO₂, C(O)(SR¹³), SR⁵, or SSR⁴; and
Y is not CN, N₂H₂R¹³, N₂HR¹³R¹⁴, N₃, SCN, SC(═NH)N(R¹⁵)₂, SC(═NH)NHR¹⁵, SC(O)N(R¹⁵)₂, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R⁵, C(O)R¹², C(O)(SR¹³), SR⁴, SR⁵, or SSR⁵, or Y does not exist.

In another embodiment, the compound contains at least 2 nitrate groups. In another embodiment, the nitrate is beta or gamma to said sulfur atom.

In another embodiment of the sixth aspect, the compound can be any of the compounds that were cited individually as an embodiment of either the first or second aspect of the invention.

In another embodiment, the compound is:

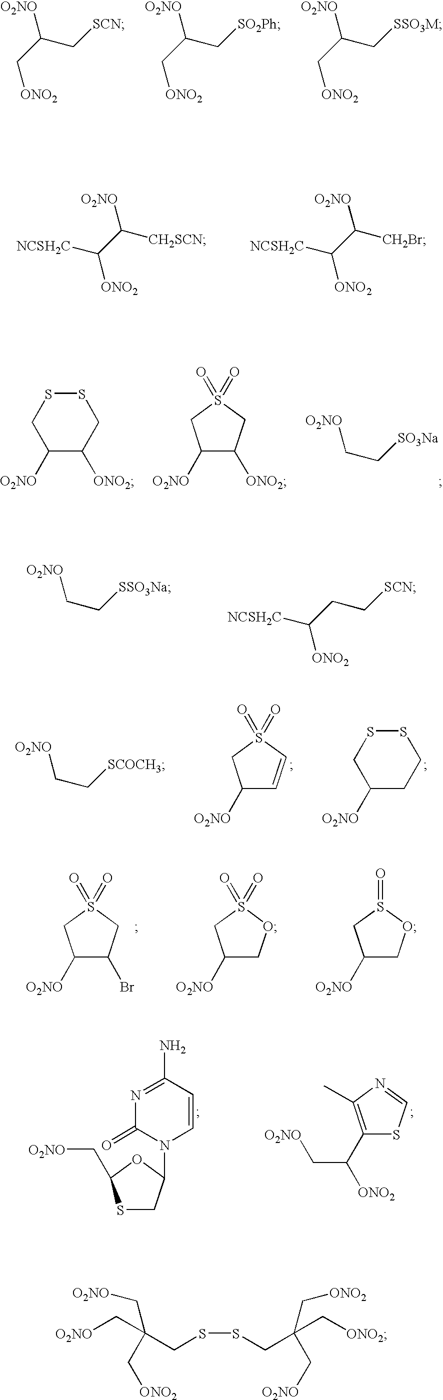

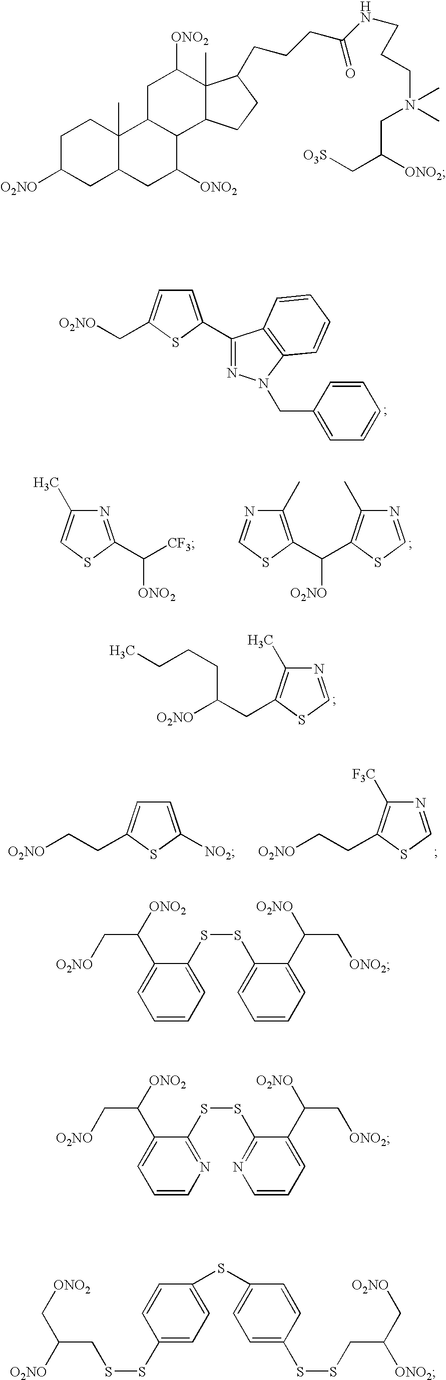

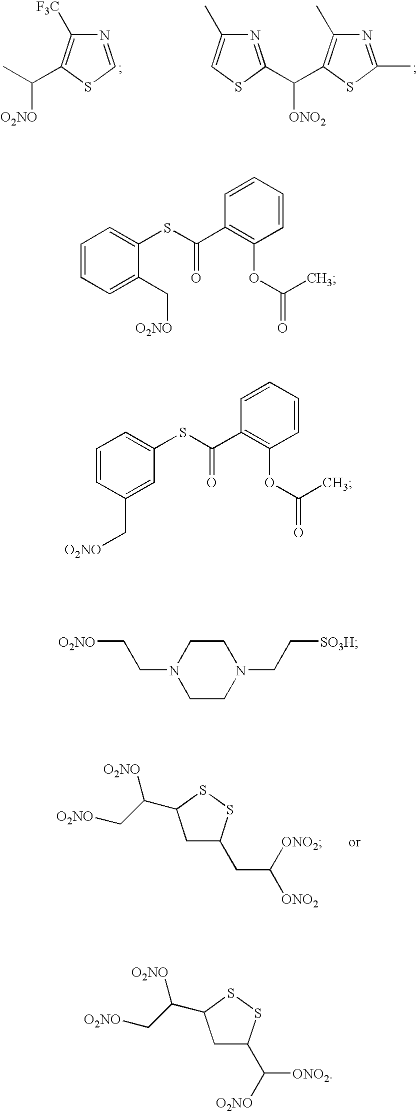

In yet another embodiment of the sixth aspect, the compound is:

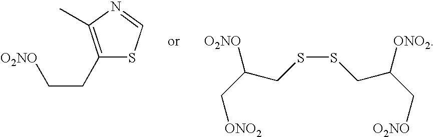

As used herein, the terms “alkyl” and the prefix “alk-” are inclusive of both straight chain and branched chain saturated or unsaturated groups, and of cyclic groups, i.e., cycloalkyl and cycloalkenyl groups. Unless otherwise specified, acyclic alkyl groups are from 1 to 6 carbons and contain at least one C—H bond. The number of carbons in an alkyl group refers to the total number of carbons contained in the group. Cyclic groups can be monocyclic or polycyclic and preferably have from 3 to 8 ring carbon atoms. Exemplary cyclic groups include cyclopropyl, cyclopentyl, cyclohexyl, and adamantyl groups. Unless otherwise indicated, alkyl groups may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “aryl” is meant a carbocyclic aromatic ring or ring system. Unless otherwise specified, aryl groups are from 6 to 18 carbons. Examples of aryl groups include phenyl, naphthyl, biphenyl, fluorenyl, and indenyl groups.

By “heterocycle” is meant an aromatic or non-aromatic ring system that contains at least one ring heteroatom (e.g., O, S, N). The term “heteroaryl” refers to an aromatic heterocyclic ring or ring system that contains at least one ring heteroatom (e.g., O, S, N). Unless otherwise specified, heteroaryl rings contain from 1 to 9 carbons. Exemplary heteroaryl groups include furanyl, thienyl, pyrrolyl, imidazolyl, pyrazolyl, oxazolyl, isoxazolyl, thiazolyl, isothiazolyl, triazolyl, oxadiazolyl, oxatriazolyl, pyridyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl rings or ring systems.

Unless otherwise specified, non-aromatic heterocyclic groups are from 2 to 9 carbons and can include, for example, dihydropyrrolyl, tetrahydropyrrolyl, piperazinyl, pyranyl, dihydropyranyl, tetrahydropyranyl, dihydrofuranyl, tetrahydrofuranyl, dihydrothiophene, tetrahydrothiophene, and morpholinyl groups. As defined in the present application, the definition of heterocycle specifically excludes β-lactam rings.

Unless otherwise specified, aryl, heteroaryl, or heterocyclic groups may be unsubstituted or substituted by one or more substituents selected from the group consisting of C₁₋₆ alkyl, hydroxy, halo, nitro, C₁₋₆ alkoxy, C₁₋₆ alkylthio, trifluoromethyl, C₁₋₆ acyl, arylcarbonyl, heteroarylcarbonyl, nitrile, C₁₋₆ alkoxycarbonyl, arylalkyl (wherein the alkyl group has from 1 to 6 carbon atoms) and heteroarylalkyl (wherein the alkyl group has from 1 to 6 carbon atoms).

By “halide” or “halogen” or “halo” is meant bromine, chlorine, iodine, or fluorine. As used herein, the terms “alkyl” and the prefix “alk-” are inclusive of both straight chain and branched chain saturated or unsaturated groups, and of cyclic groups, i.e., cycloalkyl and cycloalkenyl groups. Unless otherwise specified, acyclic alkyl groups are from 1 to 6 carbons. Cyclic groups can be monocyclic or polycyclic and preferably have from 3 to 8 ring carbon atoms. Exemplary cyclic groups include cyclopropyl, cyclopentyl, cyclohexyl, and adamantyl groups. Alkyl groups may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups.

By “alkoxy” is meant a chemical substituent of the formula —OR, where R is an alkyl group. By “aryloxy” is meant a chemical substituent of the formula —OR′, where R′ is an aryl group. By “alkaryl” is meant a chemical substituent of the formula —RR′, where R is an alkyl group and R′ is an aryl group, By “alkheteraryl” is meant a chemical substituent of the formula RR″, where R is an alkyl group and R″ is a heteroaryl group.

What is meant by “aliphatic nitrate” is a nitrate group that is connected to a non-aromatic carbon.

What is meant by “antioxidant” is a compound that attenuates or prevents oxidation of a target biomolecule through the inhibition of the initiation or propagation steps that constitute oxidative and peroxidative processes.

“Mitigating neurodegeneration” as used herein involves affecting neuroprotection, inhibiting or preventing neurodegeneration, and/or ameliorating the manifestations or impact of neurodegeneration. Such amelioration includes effecting cognition enhancement, as is quantified by tests known in the art (e.g., Venault et al., 1992, incorporated herein by reference). “Modulating” a biological process as used herein (for example, modulating activity of the non-glutamate neuroreceptors), encompasses both increasing (positively modulating) and decreasing (negatively modulating) such activity, and thus inhibition, potentiation, agonism, and antagonism of the biological process.

It is an object of the present invention to provide provide novel organic nitrates, including aliphatic nitrate esters bearing a sulfur or phosphorus moiety β or γ to a nitrate group, or congeners thereof. Another object of the present invention is to provide methods for making such novel organic nitrates. Another object of the invention is to provide methods for affecting neuroprotection, mitigating neurodegeneration, affecting cognition enhancement, and/or protecting tissues from oxidative injury employing selected organic nitrates. Another object of the present invention is to provide novel drugs as neuroprotective agents. Another another object of the present invention is to provide novel drugs for use in cognition enhancement. Another object of the invention is to provide novel drugs for use in protecting tissues from oxidative injury.

It will be understood by those skilled in the art, that when organic radicals, such as Rⁿ, X, or Y, are represented as “does not exist,” that the valency of the carbon bound to the radical is adjusted accordingly (i.e., by a point of unsaturation).

Compounds may be constructed according to formulas of the invention in which an S atom is appropriately proximally placed with respect to a nitrate functional group, but in which a carbonyl group intervenes to form a thioester linkage. Such compounds do not form part of the invention if cleavage of this thioester bond produces two entirely separate molecules, one containing the nitrate functionality, and another the S-functionality. An example of such a compound is one containing the motif, O₂NOCH₂C(CH₃)₂C(O)S-(organic radical), which would liberate on thioester cleavage an aliphatic nitrate that does not contain a S-functionality. It is understood by one skilled in the art that facile cleavage of a thioester in an aqueous biological milieu will render an aliphatic nitrate that does not contain a proximal S, and therefore, such a thioester does not comprise part of the current invention.

### BRIEF DESCRIPTION OF THE DRAWINGS

FIG. 1 is a synthetic scheme showing the use of Bunte salt IVd in the preparation of compounds Va, IVr, Vbb, and Vbc.

FIG. 2 is a graph showing a comparison of 50 μM GTN (stippled bars), 10 μM Va (cross-hatched bars), 30 μM Va (upward hatched bars) and 50 μM Va (downward hatched bars) on lactate dehydrogenase (LDH) release from isolated perfused rat heart after left coronary artery occlusion (LCAO) for 45 min followed by reperfusion for 90 min. Hearts were perfused with drugs for ten min prior to and throughout the period of LCAO. Data are the mean±standard errors (n=3−6).

FIG. 3 is a graph showing a comparison of 50 μM GTN (stippled bars), 50 μM Va (hatched bars) and 50 μM compound IIIam (cross-hatched bars) on lactate dehydrogenase (LDH) release from isolated perfused rat heart after left coronary artery occlusion (LCAO) for 45 min followed by reperfusion for 90 min. Hearts were perfused with drugs for ten min prior to and throughout the period of reperfusion. Data are the mean±standard errors (n=4−6).

FIG. 4 is a graph showing a comparison of 50 μM GTN (open bar) and 50 μM Va (hatched bar) on infarct size in isolated perfused rat heart after left coronary artery occlusion (LCAO) for 45 min followed by reperfusion for 90 min. Hearts were perfused with drugs for ten min prior to and throughout the period of LCAO. Data are the mean±standard errors (n=5−6).

FIG. 5 is a graph showing a comparison of 50 μM GTN (open bar), 50 μM Va (hatched bar) and 50 μM compound IIIam (cross-hatched bar) on infarct size in isolated perfused rat heart after left coronary artery occlusion (LCAO) for 45 min followed by reperfusion for 90 min. Hearts were perfused with drugs for ten min prior to and throughout the period of reperfusion. Data are the mean±standard errors (n=3-6).

FIG. 6 is a graph showing a comparison of 50 μM GTN (stippled bars) and 50 μM Va (hatched bars) on coronary perfusion pressure in isolated perfused rat heart after left coronary artery occlusion (LCAO) for 45 min followed by reperfusion for 90 min. Hearts were perfused with drugs for ten min prior to and throughout the period of LCAO. Coronary perfusion pressure was recorded just prior to LCAO (baseline), at the beginning of reperfusion (0 min reperfusion) and at the end of the 90 min reperfusion period (90 min reperfusion). Data are the mean±standard errors (n=6).

FIG. 7 is an immunocytochemical analysis of tyrosine hydroxylase immunoreactivity in the substantia nigra of rats. FIG. 7A is an analysis of a brain section in which the animal was administered the neurotoxic compound 6-hydroxydopamine (6-OHDA). FIG. 7B is an analysis of a brain section in which the animal was administered only vehicle control (dimethyl sulfoxide). FIG. 7C is an analysis of a brain section in which the animal was treated with compound Va before and after administration of 6-OHDA. FIG. 7D is an analysis of a brain section in which the animal was administered only vehicle control.

FIG. 8 is a graph showing the inhibition and potentiation of lipid peroxidation induced by FeSO₄ (50 μM) as assessed by TBARS (thiobarbituric acid reactive substances) determination, on incubation of rat brain synaptosomes with ascorbic acid or α-tocopherol (n=3). Percentage TBARS detected is given relative to TBARS in presence of FeSO₄ (100%). “Control” experiment is in the presence of vehicle and absence of FeSO₄. Error bars show S.E.M.

FIG. 9 is a graph showing the inhibition of lipid peroxidation induced by FeSO₄ (50 μM) as assessed by TBARS determination, on incubation of rat brain synaptosomes with Trolox (n=3). Percentage inhibition is given relative to: control TBARS in presence of vehicle and absence of FeSO₄ (100%); TBARS in presence of vehicle and FeSO₄ (0%). Error bars show S.E.M; data is fitted to a sigmoidal curve (EC₅₀=6.8×10⁻⁵ M).

FIG. 10 is a graph showing the inhibition of lipid peroxidation induced by FeSO₄ (50 μM) as assessed by TBARS determination, on incubation of rat brain synaptosomes with lipoic acid (LA, dashed line) or dihydrolipoic acid (LAH₂, solid line) (n=3). Percentage inhibition relative to: control TBARS in presence of vehicle and absence of FeSO₄ (100%); TBARS in presence of vehicle and FeSO₄ (0%). Error bars show S.E.M.

FIG. 11 is a graph showing the inhibition of lipid peroxidation induced by FeSO₄ (50 μM) as assessed by TBARS determination, on incubation of rat brain synaptosomes with: GTN (varied)+LAH₂ (1 mM) (▪, solid line); Va (▾, dashed line); or Va (varied)+LAH₂ (1 mM) (▴, dashed line), (n=3). Percentage inhibition relative to: control TBARS in presence of vehicle and absence of FeSO₄ (100%); TBARS in presence of vehicle, adjuvants and FeSO₄ (0%). In experiments with adjuvant LAH₂, 0% inhibition corresponds to TBARS in presence of vehicle containing LAH₂. Error bars show S.E.M.

FIG. 12 is a graph showing the inhibition of lipid peroxidation induced by FeSO₄ (50 μM) as assessed by TBARS determination, on incubation of rat brain synaptosomes with: Va (varied)+cysteine (1 mM) (▪, solid line); or Va+PhSH (1 mM) (▴, solid), (n=3). For comparison, inhibition curve with NONOate, DETA/NO (in the absence of adjuvants; ●, dashed line) is shown. Percentage inhibition relative to controls: TBARS in presence of vehicle and absence of FeSO₄ (100%); TBARS in presence of vehicle, adjuvant and FeSO₄ (0%). In both experiments with adjuvant thiols, 0% inhibition corresponds to TBARS in presence of vehicle containing thiol (1 mM). Error bars show S.E.M.

FIG. 13 is a graph showing the inhibition of lipid peroxidation induced by FeSO₄ (50 μM) as assessed by TBARS determination, on incubation of rat brain synaptosomes with: IVr (varied) (▪); or Va+PhSH (1 mM) (▴), (n=3). Percentage inhibition relative to: control TBARS in presence of vehicle and absence of FeSO₄ (100%); TBARS in presence of vehicle, adjuvant, and FeSO₄ (0%). In experiments with thiol, 0% inhibition corresponds to TBARS in presence of vehicle containing adjuvant PhSH. Error bars show S.E.M.

FIG. 14 is a graph showing the effects of NO-donors. Inhibition of lipid peroxidation induced by FeSO₄ (50 μM) as assessed by TBARS determination, on incubation of rat brain synaptosomes with: (a) DEA/NO (diethylamine NONOate, ▪, solid line); and Sper/NO (spermine NONOate, □, dashed lines), (n=3). Percentage inhibition relative to: control TBARS in presence of vehicle, adjuvant, and absence of FeSO₄ (100%); TBARS in presence of vehicle and FeSO₄ (0%). Error bars show S.E.M. Data is fitted to sigmoidal curves (EC₅₀: DEA/NO=7×10⁻⁵ M; Sper/NO=2×10⁻⁴ M).

FIG. 15 is a graph showing the inhibition of lipid peroxidation induced by FeSO₄ (50 μM) as assessed by TBARS determination, on incubation of rat brain synaptosomes with i-amyl nitrite (IAN, ▪ solid line) (n=3). Percentage inhibition relative to: control TBARS in presence of vehicle and absence of FeSO₄ (100%); TBARS in presence of FeSO₄ (0%). Data is fitted to a sigmoidal curve: EC₅₀=1.6×10⁻⁴ M.

FIG. 16 is a graph showing the comparison of inhibition by nitrate IVs in: (1) FeSO₄ (50 μM) induced lipid peroxidation in rat brain synaptosomes assessed by TBARS determination (◯ dashed line); and, (2) ABAP induced lipid peroxidation in liposomes assessed by BODIPY peroxidation (● solid line) (n=3). Percentage inhibition relative to: control response in presence of vehicle and absence of initiator (100%); response in presence of vehicle and initiator (0%). Data is fitted to a sigmoidal curve: EC₅₀ (synaptosomes)=1.0×10⁻³ M, (liposomes)=1.3×10⁻⁴ M.

FIG. 17 is a graph showing the comparison of inhibition of lipid peroxidation induced by FeSO₄ (50 μM) as assessed by TBARS determination, on incubation of rat brain synaptosomes with nitrate Vbb (▾ solid line) and nitrate Vbc (▪ solid line), compared to Trolox (□ dashed line) (n=3). Percentage inhibition relative to: control TBARS in presence of vehicle and absence of FeSO₄ (100%); TBARS in presence of FeSO₄ (0%). Data is fitted to a sigmoidal curve: EC₅₀ Vbb=2.0×10⁻⁵ M; EC₅₀ Vbc=7×10⁻⁷ M and 7×10⁻⁵ M).

### DETAILED DESCRIPTION OF THE INVENTION

This invention pertains to methods and compositions useful for treating neurodegeneration or preventing or mitigating tissue and/or cellular damage by administering to a subject a therapeutic nitrate ester. Neuroprotection and/or cognition enhancement can be affected, for example, by modulating an interaction with guanylyl cyclase (GCase, the enzyme responsible for cGMP production in various areas of the brain), modulating a glutamate or non-glutamate neuroreceptor or attenuating free radical damage. The attenuation of free radical concentration by a nitrate ester of the invention can also be useful for preventing or mitigating tissue and/or cellular damage.

According to certain aspects of the invention, neurodegeneration is mitigated by stimulating cerebral GCase. One of the major targets for the novel organic nitrates of the invention is GCase activation, resulting in the production of cGMP. Experimental evidence obtained in a number of in vitro model systems supports the notion that elevated levels of cGMP help to prevent apoptotic (programmed) cell death. Thus, a cGMP-dependent mechanism significantly increases the survival of trophic factor-deprived PC12 cells and rat sympathetic neurons (Farinelli et al., 1996), and of primary cultures of rat embryonic motor neurons (Estevez et al., 1998). The mechanism of action for selected organic nitrates in preventing apoptotic cell death may be inhibition of caspase-3 activation indirectly through elevations in cGMP levels or directly via protein S-nitrosylation of the enzyme by an NO-intermediate (Kim et al., 1997). Caspase-3 is a member of the cysteine protease family of enzymes that are essential for the execution step in apoptosis (Cohen, 1997; Nicholson and Thornberry, 1997). Activation of caspase-3 is required for apoptotic cell death in trophic factor-deprived PC12 cells (Haviv et al., 1997) and in glutamate-mediated apoptotic cell death of cultured cerebellar granule neurons (Du et al., 1997). In animal models of cerebral ischemia, caspase-3 activity is induced and may be responsible for the apoptotic component of delayed neuronal cell death (Chen et al., 1998; Namura et al., 1998; Ni et al., 1998). Inhibitors of caspase-3 significantly decrease the apoptotic component of delayed neuronal cell death in response to ischemic injury both in vitro (Gottron et al., 1997) and in vivo (Endres et al., 1998). A secreted region of the Alzheimer's disease β-amyloid precursor protein lowers intracellular calcium levels and provides neuroprotective effects on target cells through increases in cGMP levels and activation of protein kinase G (Barger et al., 1995; Furukawa et al., 1996). In preferred embodiments of the methods of the invention, nitrated molecules that have the capacity to activate GCase directly or via release of an NO-containing intermediate are used to modulate GCase activity.

According to certain other aspects of the invention, cognition enhancement (e.g., improved memory performance) is achieved by stimulating cerebral GCase. Several lines of experimental evidence support the notion that GCase and cGMP are involved in the formation and retention of new information. cGMP has been directly implicated in both long-term potentiation (LTP) and long-term depression (LTD), which are proposed cellular models for learning and memory (Arancio et al., 1995; Wu et al., 1998). In animal models, elevation of hippocampal cGMP levels leading to increased protein kinase G activity has been shown to be important for retention and consolidation of new learning (Bernabeu et al., 1996, 1997). Thus, stimulation of cerebral GCase activity is expected to improve learning and memory performance in individuals in whom cognitive abilities are impaired by injury, disease, or aging.

We have shown that novel organic nitrate esters have differential effects to activate soluble GCase and to cause cGMP accumulation in vascular and brain tissue. There is a clear dissociation between the vascular relaxation effects of organic nitrate esters and ability to affect neuroprotection. Activation of GCase and accumulation of cGMP have been shown to be important in the neuroprotection of hippocampal brain slices subjected to a period of in vitro ischemia.

Cerebral ischemia results in marked increases in the release of the excitatory amino acid glutamate in the affected brain region (Bullock et al., 1998; Huang et al., 1998; Yang et al., 1998). In both humans (Bullock et al., 1998) and experimental animals (Huang et al., 1998; Goda et al., 1998; Yang et al., 1998), the amount of glutamate released during ischemia is positively correlated with the extent of brain injury. In experimental animal models of cerebral ischemia, decreased release of glutamate during ischemia (Goda et al., 1998) or blockade of glutamate receptors with antagonists (Ibarrola et al., 1998; O'Neill et al., 1998; Umemura et al., 1997) significantly reduces the extent of brain injury. However, these interventions are only effective when given prior to or during the ischemic insult. To be broadly useful, a therapeutic intervention is preferably effective when administered after the period of ischemia. We have designed a class of novel organic nitrate esters having high efficacy in effecting neuroprotection in vivo in models of transient global and focal cerebral ischemia when given after the ischemic insult. It will be appreciated, therefore, that these selected organic nitrates can be used for treatment of conditions including but not limited to: stroke; Parkinson's disease; Alzheimer's disease; Huntington's disease; multiple sclerosis; amylotrophic lateral sclerosis; AIDS-induced dementia; epilepsy; alcoholism; alcohol withdrawal; drug-induced seizures; viral/bacterial/fever-induced seizures; trauma to the head; hypoglycemia; hypoxia; myocardial infarction; cerebral vascular occlusion; cerebral vascular hemorrhage; hemorrhage; environmental excitotoxins of plant, animal, or marine origin; and the like.

The direct effects of selected organic nitrates on amino acid neurotransmitter receptors has been tested using the Xenopus oocyte expression system and two-electrode voltage-clamp recording methods. Selected organic nitrates were found to have direct, modulatory effects on GABA_{A} receptor function (see Working Examples below). These allosteric modulatory effects of selected organic nitrates were not shared by direct NO-generating compounds, indicating a novel mechanism of action for selected organic nitrates to interact with GABA_{A} receptors. In behavioural models of learning and memory, drugs which decrease GABA_{A} receptor function improve performance on learning and memory tasks (Venault et al., 1992). Thus, the behavioural effect of selected organic nitrates, developed to act as modulators of GABA_{A} receptor function, will be to improve memory performance and cognition in patient populations. It will be appreciated, therefore, that these selected organic nitrates can be used for treatment of conditions including but not limited to: stroke; dementias of all type; trauma; drug-induced brain damage; and aging.

According to certain aspects of the invention, neurodegeneration is mitigated by inhibition of free radical damage. Reoxygenation and reperfusion after a period of ischemia contributes significantly to the development of brain injury. Oxygen radicals, especially superoxide and peroxynitrite, formed in the period after an ischemic event may initiate processes such as breakdown of membrane lipids (lipid peroxidation), leading to loss of cell membrane integrity and inhibition of mitochondrial function (Macdonald and Stoodley, 1998; Gaetani et al, 1998). Oxidative stress is also believed to be one factor involved in initiation of apoptotic neuronal cell death (Tagami et al., 1998). In experimental animal models of ischemic brain injury, free radical scavengers and enhanced activity of superoxide dismutase have been found to reduce the extent of neuronal injury and cell death (Chan et al., 1998; Mizuno et al., 1998; Tagami et al., 1998).

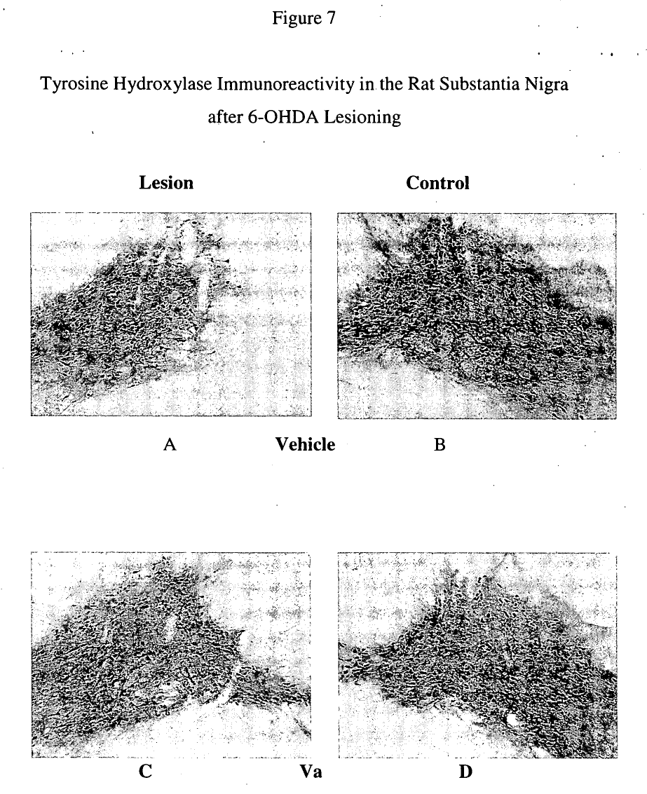

It has been shown that 2,3-dinitrooxy-(2,3-bis-nitrooxy-propyldisulfanyl)-propane, compound Va, improves task acquisition in cognitively impaired animals (see Smith et al., NeuroReport 11: 3883, 2000), suggesting to us that novel nitrates administered to an intact animal can have direct effects on the brain. These observations also lead us to postulate that the nitrates of the present invention could protect tissues against oxidative injury. Accordingly, we tested the effects of compound Va in the 6-hydroxydopamine (6-OHDA) model of Parkinson's disease. 6-OHDA is a neurotoxin selectively taken up into dopaminergic neurons, resulting in a selective killing of these neurons, via a mechanism involving oxidative stress that is evident by 4 days after injection of the toxin. Previous studies have demonstrated that the monoamine oxidase type B (MAO-B) inhibitor, deprenyl, can prevent 6-OHDA-induced killing of dopaminergic neurons. We therefore employed deprenyl as a positive control in this study. Loss of dopaminergic innervation to the striatum results in an upregulation of postsynaptic dopamine receptors, and the development of dopamine receptor supersensitivity 2-3 weeks after 6-OHDA lesioning of the substantia nigra. This supersensitivity to dopamine can be unmasked by challenging the animal with a dopamine receptor agonist, such as apomorphine. Since the substantia nigra pars compacta is a bilateral structure, unilateral destruction of the substantia nigra will induce a dopamine receptor imbalance, which can manifest behaviourally as apomorphine-induced turning in the direction contralateral to the lesion. This behavioural manifestation of 6-OHDA toxicity provides a convenient, quantifiable index of neuronal injury and neuroprotection. Immunocytochemical analysis confirmed that compound Va preserved TH-positive neurons in the substantia nigra of 6-OHDA-injected rats (see Example 3 and FIG. 7). These data demonstrate that compound Va is a very effective neuroprotective agent against 6-OHDA-induced killing of dopaminergic neurons in the rat substantia nigra pars compacta.

Accordingly, in certain aspects and embodiments of the invention, the invention features nitrated molecules which have the capacity to inhibit production of free radicals and/or which act as free radical scavengers.

Free radical overproduction is associated with a wide range of disease states and/or indications, such as, for example, aging, septic shock, ischemia, overexpression of cytokines, ulcers, inflammatory bowel disease (e.g., gastritis, ulcerative colitis or Crohn's disease), diabetes, arthritis (e.g., rheumatoid arthritis), asthma, cirrhosis, allograft rejection (e.g., transplant rejection), encephalomyelitis, meningitis, pancreatitis, peritonitis, vasculitis, lymphocytic choriomeningitis, glomerulonephritis, ophthalmologic diseases (e.g., uveitis, glaucoma, blepharitis, chalazion, allergic eye disease, corneal ulcer, keratitis, cataract, retinal disorders, age-related macular degeneration, optic neuritis, and the like), ileitis, inflammation induced by overproduction of inflammatory cytokines (e.g., liver inflammation, renal inflammation, airway inflammation, and the like), hemorrhagic shock, anaphylactic shock, burn, infection leading to the overproduction of inflammatory cytokines (including bacterial (e.g., E. coli infection), viral (e.g., HIV), fungal (e.g., Candidiosis and histoplasmosis) and parasitic (e.g., Leishmaniasis and Schistosomiasis) infections), hemodialysis, chronic fatigue syndrome, stroke, cancers, including metastatic cancers (e.g., breast cancer, bladder cancer, lung cancer, colon cancer, or cancer of the other organs, or skin or other noncutaneous portions of the body), cardiovascular diseases associated with overproduction of inflammatory cytokines (e.g., heart disease, cardiopulmonary bypass, ischemic/reperfusion injury, and the like), ischemic/reperfusion associated with overproduction of inflammatory cytokines, toxic shock syndrome, adult respiratory distress syndrome, cachexia, myocarditis, autoimmune disorders, eczema, psoriasis, heart failure, dermatitis, urticaria, cerebral ischemia, systemic lupus erythematosis, AIDS, neurodegenerative disorders (e.g., chronic neurodegenerative disease), chronic pain, priapism, cystic fibrosis, schizophrenia, depression, premenstrual syndrome, anxiety, addiction, migraine, gastrointestinal motility disorders, obesity, hyperphagia, solid tumors (e.g., neuroblastoma), malaria, hematologic cancers, myelofibrosis, lung injury, graft-versus-host disease, head injury, CNS trauma, hepatitis, renal failure, liver disease (e.g., chronic hepatitis C), drug-induced lung injury (e.g., paraquat), transplant rejection and preservation, fertility enhancement, bacterial translocation, circulatory shock, traumatic shock, and vascular aneurysm (e.g., aortic aneurysm), ileus, or myocardial infarction.

In addition, the compounds or methods of the present invention may find use in cytokine therapy (with consequent induction of free radical overproduction) which, for example, is commonly used in the treatment of cancers, including metastatic cancers (e.g., breast cancer, bladder cancer, lung cancer, colon cancer, or cancer of the other organs, or skin or other noncutaneous portions of the body), autoimmune disease, and in AIDS patients. Systemic hypotension due to the induction of free radical overproduction is a dose-limiting side effect of cytokine therapy. Thus, a large patient population exists which will benefit from the invention methods.

It will also be appreciated by those skilled in the art, that any organic nitrate in which vasodilatory potency is reduced and neuroprotective potency increased, represents a new and useful therapeutic agent for use in neuroprotection, particularly in treatment of conditions including but not limited to: stroke; Parkinson's disease; Alzheimer's disease; Huntington's disease; multiple sclerosis; amylotrophic lateral sclerosis; AIDS-induced dementia; epilepsy; alcoholism; alcohol withdrawal; drug-induced seizures; viral/bacterial/fever-induced seizures; trauma to the head; hypoglycemia; hypoxia; myocardial infarction; cerebral vascular occlusion; cerebral vascular hemorrhage; hemorrhage; environmental excitotoxins of plant, animal, or marine origin. GTN itself, proposed as a neuroprotective agent, has no clinical utility as a neuroprotective agent in therapy owing to its extraordinarily high vasodilatory potency. Similarly, by extrapolation, 1,2,3-trinitratopropane (GTN) derivatives are not expected to have clinical utility as neuroprotective agents in therapy owing to their especially high vasodilatory potency.

It will additionally be appreciated by those skilled in the art, that the use in therapy of any organic nitrate in cognition enhancment, represents a new and useful treatment for cognition enhancement, particularly in treatment of conditions including but not limited to: stroke; dementias of all type, trauma, drug-induced brain damage, and aging.

In particular, the therapeutic compounds of the invention comprise at least one nitrate group. The nitrate groups(s) can optionally be covalently bound to a carrier moiety or molecule (e.g., an aromatic group, an aliphatic group, peptide, steroid, nucleoside, peptidomimetic, steroidomimetic, or nucleoside analogue, or the like). In addition to functioning as a carrier for the nitrate functionality, the carrier moiety or molecule can enable the compound to traverse biological membranes and to be biodistributed preferentially, without excessive or premature metabolism. Further, in addition to functioning as a carrier for the nitrate functionality, the carrier moiety or molecule can enable the compound to exert amplified neuroprotective effects and/or cognition enhancement through synergism with the nitrate functionality.

In one aspect, the invention provides a method of treating a neurological condition and/or preventing an undesirable mental condition (e.g., memory loss) including the step of administering to a subject an effective amount of a therapeutic compound capable of mitigating neurodegeneration which has at least one nitrate group. In one embodiment, the therapeutic compound is capable of effecting neuroprotection. In another embodiment of the invention, the therapeutic compound is capable of effecting cognition enhancement. The therapeutic compound has the formula (Formula I): 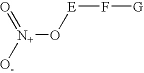
wherein E, F, G are organic radicals which may contain inorganic counterions; so that a neurological condition is treated.

In another aspect, the invention provides a pharmaceutical composition including a physiologically acceptable carrier and a compound having the formula (Formula I): 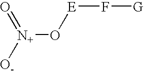
wherein: E, F, G are organic radicals which may contain inorganic counterions. The composition is employed for mitigating neurodegeneration, effecting neuroprotection and/or effecting cognition enhancement. The composition may also be employed for preventing or mitigating tissue and/or cellular damage in a subject by modulating intercellular and/or intracellular free radical concentration in the subject.

In another aspect, therapeutic compounds of the invention that effect neuroprotection and/or effect cognition enhancement in a subject to which the therapeutic compound is administered have the formula (Formula II): 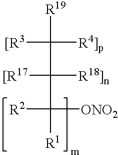
in which: m, n, p are integers from 0 to 10; R^{3,17} are each, independently, hydrogen, a nitrate group, or A; R^{1,4} are each, independently, hydrogen or A, where A is selected from: a substituted or unsubstituted aliphatic group (preferably a branched, or straight-chain aliphatic moiety having from 1 to 24 carbon atoms in the chain, which optionally contains 1 to 4 O, S, NR⁶ and/or unsaturations in the chain, optionally bearing from 1 to 4 hydroxy, nitrate, amino or aryl, or heterocyclic groups; an unsubstituted or substituted cyclic aliphatic moiety having from 3 to 7 carbon atoms in the aliphatic ring, which optionally contains 1 to 2 O, S, NR⁶, and/or unsaturations in the ring, optionally bearing from 1 to 4 hydroxy, nitrate, amino or aryl, or heterocyclic groups; an unsubstituted or substituted aliphatic moiety constituting a linkage from 0 to 5 carbons, between R¹ and R³ and/or between R¹⁷ and R⁴, which optionally contains 1 to 2 O, S, NR⁶ and/or unsaturations in the linkage, and optionally bearing from 1 to 4 hydroxy, nitrate, amino or aryl, or heterocyclic groups); a substituted or unsubstituted aliphatic group (preferably a branched, cyclic or straight-chain aliphatic moiety having from 1 to 24 carbon atoms in the chain), containing linkages selected from C═O, C═S, and C═NOH, which optionally contains 1 to 4 O, S, NR⁶, and/or unsaturations in the chain, optionally bearing from 1 to 4 hydroxy, nitrate, amino or aryl, or heterocyclic groups; a substituted or unsubstituted aryl group; a heterocyclic group; an amino group selected from alkylamino, dialkylamino, cyclic amino, cyclic diamino, cyclic triamino, arylamino, diarylamino, and alkyarylamino; a hydroxy group; an alkoxy group; and a substituted or unsubstituted aryloxy group; R², R⁵, R¹⁸, R¹⁹ are optionally hydrogen, A, or X-Y; where X is F, Br, Cl, NO₂, CH₂, CF₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂H, PO₂M, P(O)(OR¹⁴), P(O)(R¹³), SO, SO₂, C(O)(SR¹³), SR⁵, SSR⁷ or SSR⁵; Y is F, Br, Cl, CH₃, CF₂H, CF₃, OH, NH₂, NHR⁶, NR⁶R⁷, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O)R¹², C(O)(OR¹³), C(O)(SR¹³), SR⁵, SSR⁷ or SSR⁵, or does not exist; R⁶, R⁷, R⁸, R⁹, R¹¹, R¹², R¹³, R¹⁴, R¹⁵, R¹⁶ are the same or different alkyl or acyl groups containing 1-24 carbon atoms which may contain 1-4 ONO₂ substituents; or C₁-C₆ connections to R¹-R⁴ in cyclic derivatives; or are each independently hydrogen, a nitrate group, or A; M is H, Na⁺, K⁺, NH₄⁺, N⁺HₖR¹¹₍₄₋ₖ₎ where k is 0-3, or other pharmaceutically acceptable counterion.

Pharmaceutical compostions comprising a compound of Formula II in admixture with a pharmaceutically acceptable carrier therefor are provided by the invention. The invention further provides methods of mitigating neurodegeneration, effecting neuroprotection and/or effecting cognition enhancement in a subject comprising the step of administering a compound of Formula II to a subject such that said mitigation and/or said neuroprotection an/or cognition enhancement occurs.

According to this aspect of the invention, preferred therapeutic compounds for effecting neuroprotection and/or cognition enhancement in a subject to which the compound is administered include compounds in which R¹⁹ is X—Y. In some preferred embodiments: R¹⁹ is X—Y and R⁵, R⁶, R⁸, R⁹, R¹², R¹³, R¹⁴, R¹⁵, R¹⁶ are the same or different alkyl groups containing 1-24 carbon atoms which may contain 1-4 ONO₂ substituents, or C₁ or C₂ connections to R¹-R³ in cyclic derivatives; R¹ and R³ are the same or different and selected from H, C₁-C₄, alkyl chains, which may include one O, linking R¹ and R³ to form pentosyl, hexosyl, cyclopentyl, or cycohexyl rings, which rings optionally bear hydroxyl substituents; R² and R⁴, are the same or different and selected from H, a nitrate group, C₁-C₄ alkyl optionally bearing 1-3 nitrate group, and acyl groups (—C(O)R⁵); and R⁷, R¹¹ are the same or different C₁-C₈, alkyl or acyl.

In certain embodiments in which R¹⁹ is X—Y, m, p=1, and n=0. In other embodiments in which R¹⁹ is X—Y, X is selected from: CH₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³) PO₂M, P(O)(OR¹⁴), P(O)(R¹³), SO, SO₂, C(O)(SR¹³), SSR⁴. In certain other embodiments in which R¹⁹ is X—Y, Y is selected from CN, N₂H₂R¹³, N₂HR¹³R¹⁴, N₃, SCN, SC(═NH)N(R¹⁵)₂, SC(O)N(R¹⁵)₂, SC(O)NHR¹⁵, SO₃M, SR⁴, SO₂M, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O)R¹², C(O)(SR¹³), SR⁵, SSR⁵, or does not exist. In some embodiments, X and/or Y contains a sulfur-containing functional group. In certain embodiments, the compound of the invention comprises a heterocyclic functionality, more preferably, a nucleoside or nucleobase. In other embodiments, the compound of the invention comprises a carbocyclic functionality, more preferably, a steroidal or carbohydrate moiety.

In another aspect of the invention, a therapeutic compound of the invention is represented by the formula (Formula III): 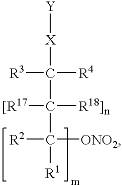
in which: m, n are 1-10; R¹⁻⁹, R¹¹⁻¹⁸, X, and Y have the meaning as defined above. In certain preferred embodiments, R⁶-R⁹ and R¹¹-R¹⁶ are the same or different alkyl or acyl groups containing 1-24 carbon atoms which may contain 1-4 ONO₂ substituents, or C₁-C6 connections to R¹-R⁴ in cyclic derivatives. In certain preferred embodiments, R¹⁸ is A and m=n=1.

Examples and preferred embodiments of this aspect include:

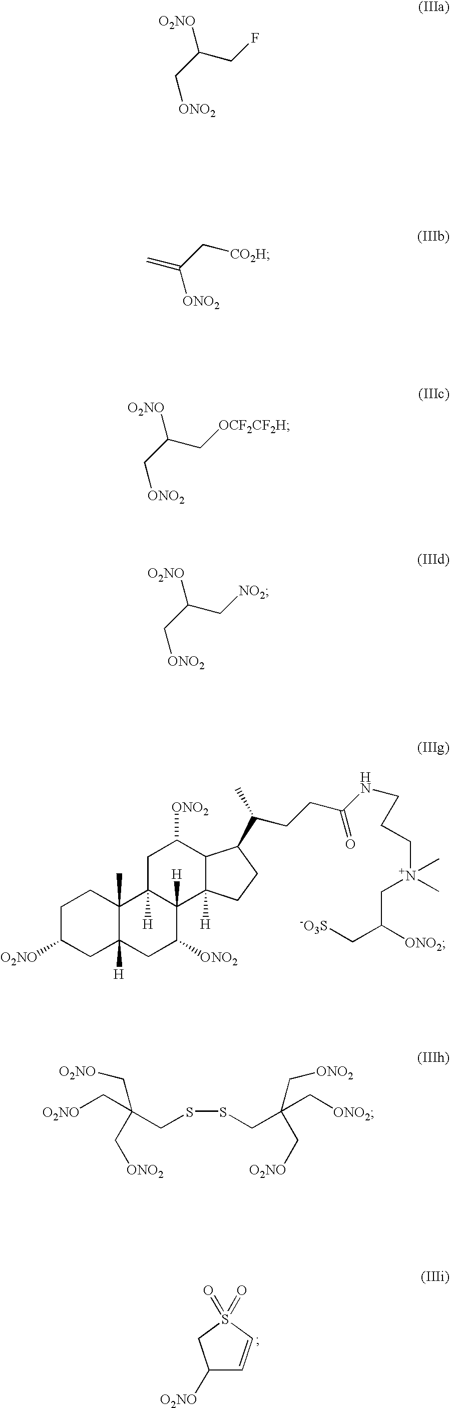

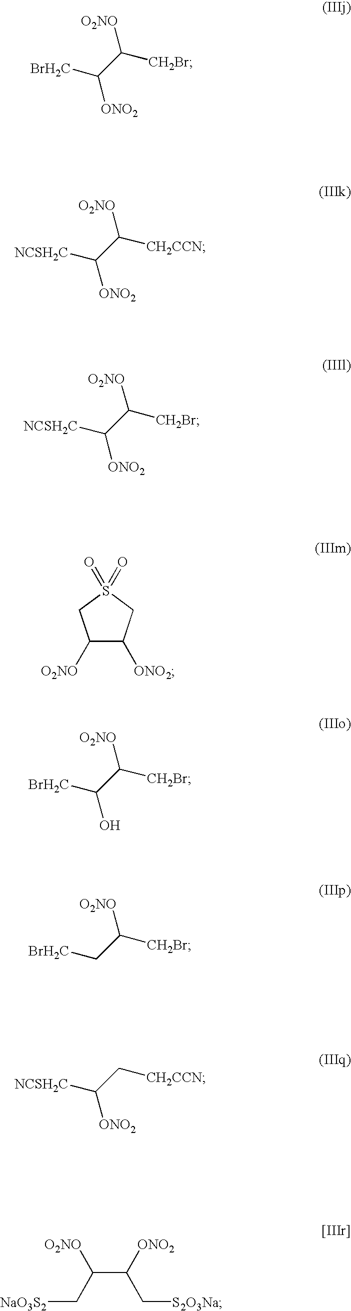

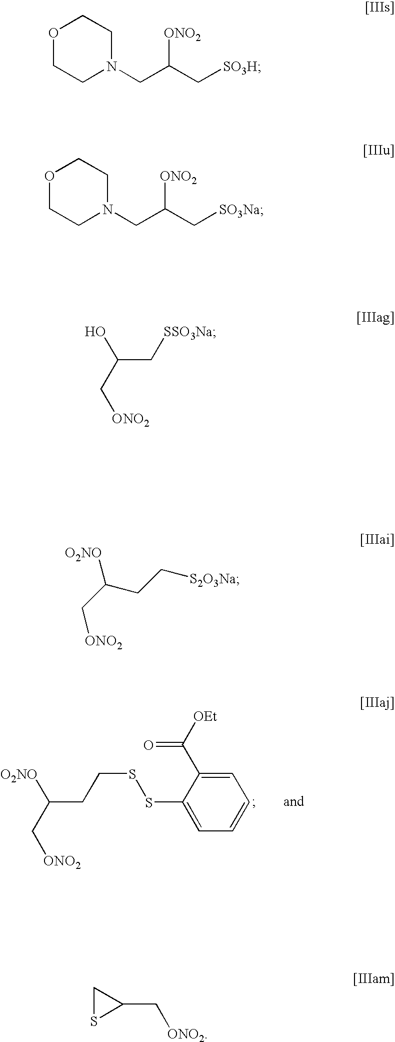

In another aspect of the invention, a therapeutic compound of the invention is represented by the formula III in which the compound contains from 1 to 3 nitrate groups and an S atom in proximity to a nitrate group, where each of m and n is, independently, an integer from 0 to 10; R¹ is a hydrogen or A; each of R², R⁵, and R¹⁸ is, independently, hydrogen or A; each of R³, R⁴, and R¹⁷, is independently, a hydrogen, a nitrate group, or A; each of R⁶, R⁷, R⁸, R⁹, R¹¹, R¹², R¹³, R¹⁴, R¹⁵, and R¹⁶ is, independently, A, a hydrogen, a nitrate group, or a C₁-C₂₄ alkyl or acyl group, optionally containing 1-4 ONO₂ substituents or a C₁-C₆ linkage to R¹, R², R³, or R⁴ in cyclic derivatives; each of R⁷ and R¹¹ is, independently, a substituted or unsubstituted C₁-C₈ alkyl or acyl group;
A is a C₁-C₂₄ alkyl group, which optionally contains 1 to 4 O, S, NR⁶, and/or unsaturations in the chain, optionally bearing from 1 to 4 hydroxy, Cl, F, amino, unsubstituted or substituted aryl, unsubstituted or substituted heteroaryl, or unsubstituted or substituted heterocyclic groups, or 1-2 nitrate groups; a C₃-C₂₄ alkyl group, containing 1-5 C═O, C═S, or C═NOR⁷ linkages, which optionally contains 1 to 4 O, S, NR⁶, and/or unsaturations in the carbon chain, optionally bearing from 1 to 4 hydroxy, nitrate, Cl, F, amino, unsubstituted or substituted aryl, unsubstituted or substituted heteroaryl, or unsubstituted or substituted heterocyclic groups; a C₃-C₇ linkage to any of R¹, R², R³, R⁴, or R¹⁷, forming an aliphatic ring, which optionally contains O, S, NR⁶, and/or unsaturations in the linkage, optionally bearing from 1 to 6 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups; a C₀-C₅ linkage to or between any of R¹, R³, R⁴, or R¹⁷, which optionally contains O, S, NR⁶, and/or unsaturations in the linkage, bearing two or more substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups; an unsubstituted C₀-C₅ linkage to or between any of R¹, R³, and R⁴, which optionally contains 1 to 2 non-adjacent O, S, NR⁶, and/or unsaturations in the linkage; a C₁-C₅ linkage to or between any of R¹, R³, R⁴, and R¹⁷ containing 1 to 2 C═O, C═S, or C═NOR⁷ linkages, which optionally contains 1 to 2 O, S, NR⁶, and/or unsaturations in the linkage, optionally bearing from 1 to 4 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups; a substituted or unsubstituted aryl group; a substituted or unsubstituted heteroaryl group; a substituted or unsubstituted heterocyclic group; an amino, cyclic amino, diamino, triamino, alkylamino, dialkylamino, arylamino, diarylamino, or alkylarylamino group; a hydroxy group; an alkoxy group; or a substituted or unsubstituted aryloxy group;
X is F, Br, NO₂, CH₂ CF₂, O, NH, NMe, NHOH, N₂H₃, N₂H₂R¹³, N₂HR¹³R¹⁴, N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)R⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷, or does not exist;
Y is F, Br, CH₃, CF₂H, CF₃, OH, NH₂, NHR⁶, NR⁶R⁷, NHOH, N₂H₃, N₂H₂R¹³, N₂HR¹³R¹⁴, N₃; SC(═NH)N(R¹⁵)₂, SC(═NH)NHR¹⁵, SC(O)N(R¹⁵)₂, SC(O)NHR¹⁵, SH, SR⁷, SO₂M, S(O)R⁸, S(O)R⁵, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R⁵, C(O)(OR¹³), C(O)(SR¹³), C(S)R⁵, C(S)R¹², C(S)OR¹², or does not exist; and
M is H, Na⁺, K⁺, NH₄⁺, N⁺HₖR¹¹₍₄₋ₖ₎ where k is 0-3, or other pharmaceutically acceptable counterion;
and with the proviso that, when m=0 and n=1, each of R¹⁸ and R³ is, independently, a hydrogen, a nitrate group, or a C₁-C₄ alkyl chain, which may include one O, linking R¹⁸ and R³ together to form a pentosyl, a hexosyl, a cyclopentyl, or a cyclohexyl ring, said ring optionally bearing from 1-4 hydroxyl substituents; each of R¹⁷ and R⁴ is, independently, a hydrogen, a nitrate group, a C₁-C₄ alkyl group, optionally bearing from 1-3 nitrate groups, or an acyl group (—C(O)R⁵); each of R⁵, R⁶, R⁸, R⁹, R¹⁰, R¹², R¹³, R¹⁴, R¹⁵, and R¹⁶ is, independently, a C₁-C₁₂ alkyl group, optionally bearing from 1-4 ONO₂ substituents, or a C₁-C₁₂ alkyl linkage to R¹⁸, R¹⁷, or R³; X is F, Br, NO₂, CH₂ CF₂, O, NH, NMe, NHOH, N₂H₃, N₂H₂R¹³, N₂HR¹³R¹⁴, N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)R⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷; and Y is not CN, N₂H₂R¹³, N₂HR¹³R¹⁴, N₃, SCN, SC(═NH)N(R¹⁴)₂, SC(═NH)NHR¹⁵, SC(O)N(R¹⁵)₂, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R⁵, C(O)R¹², C(O)(SR¹³), SR⁴, SR⁵, or SSR⁵, or Y does not exist.

Examples and preferred embodiments of this aspect include:

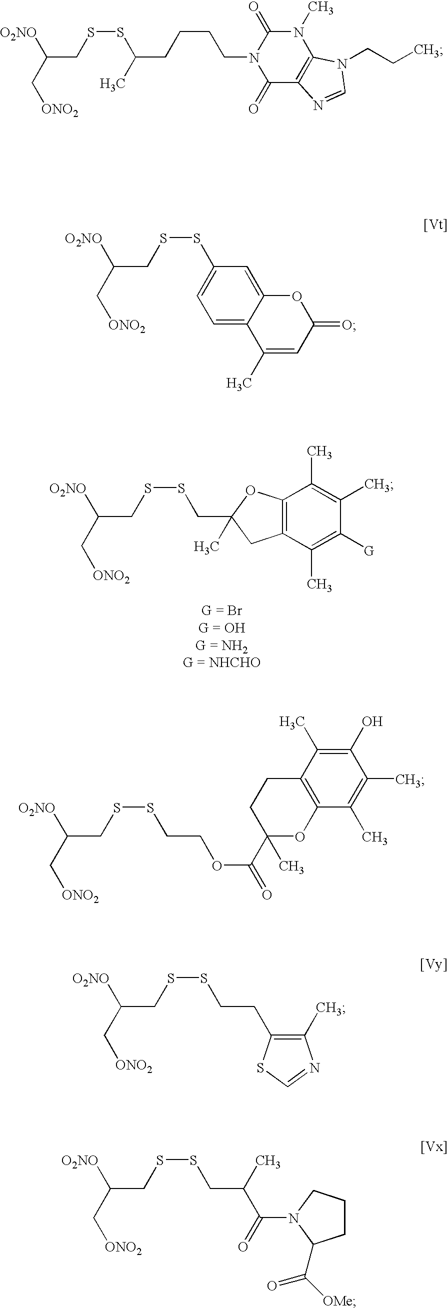

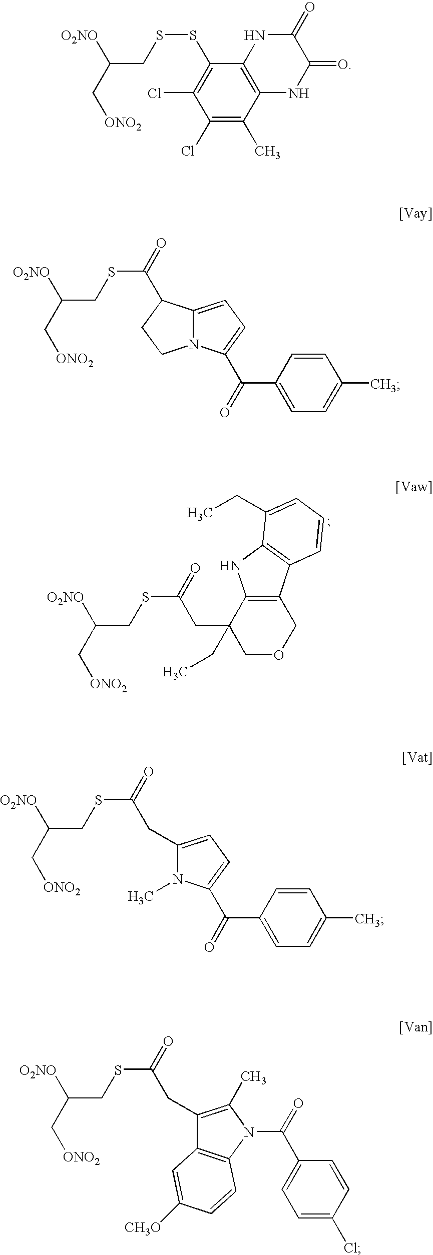

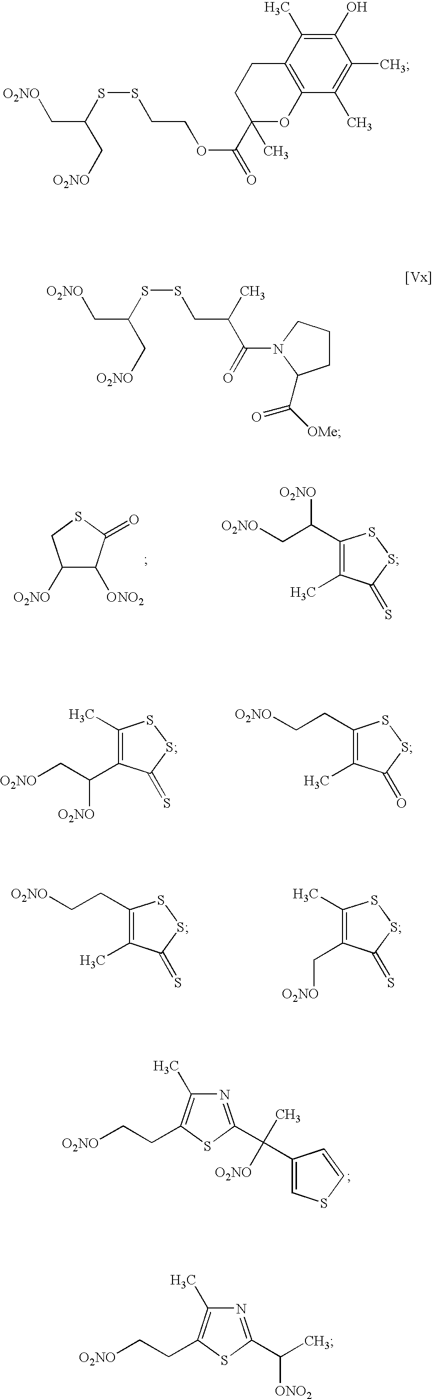

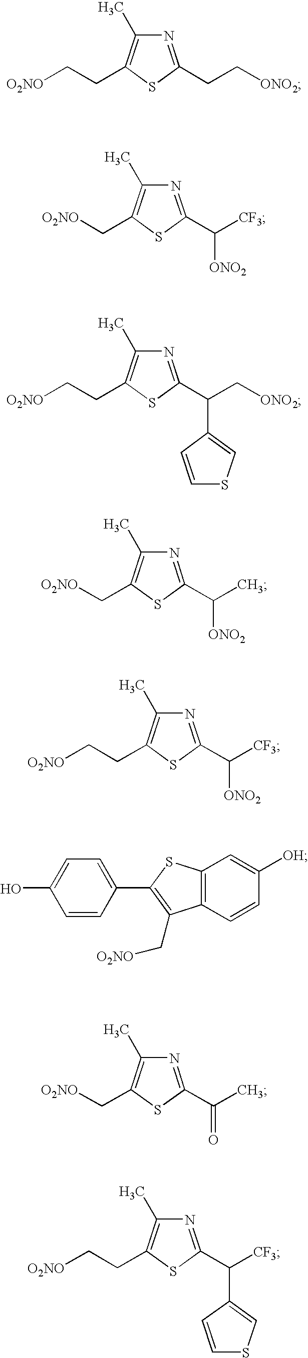

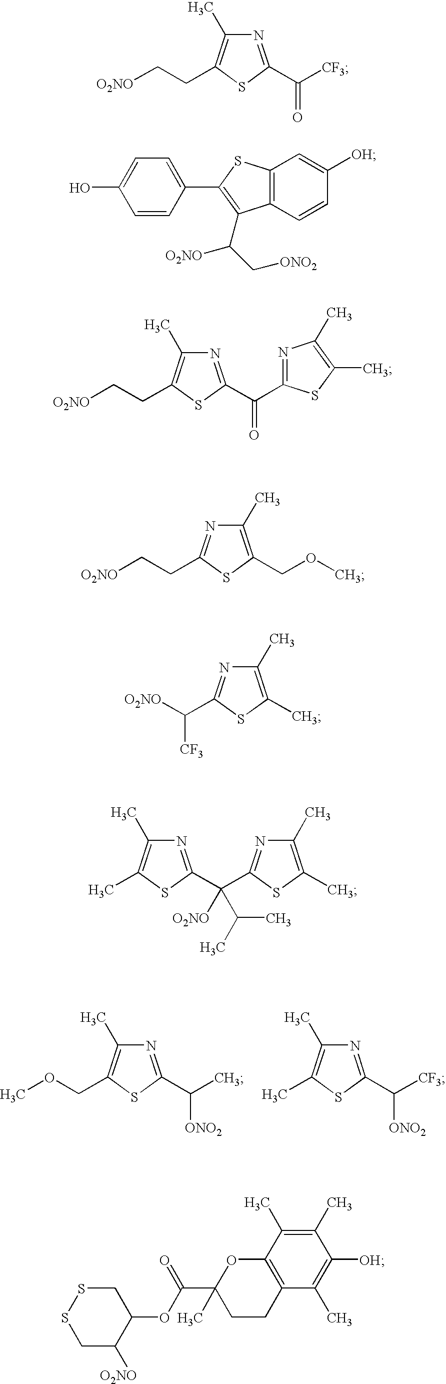

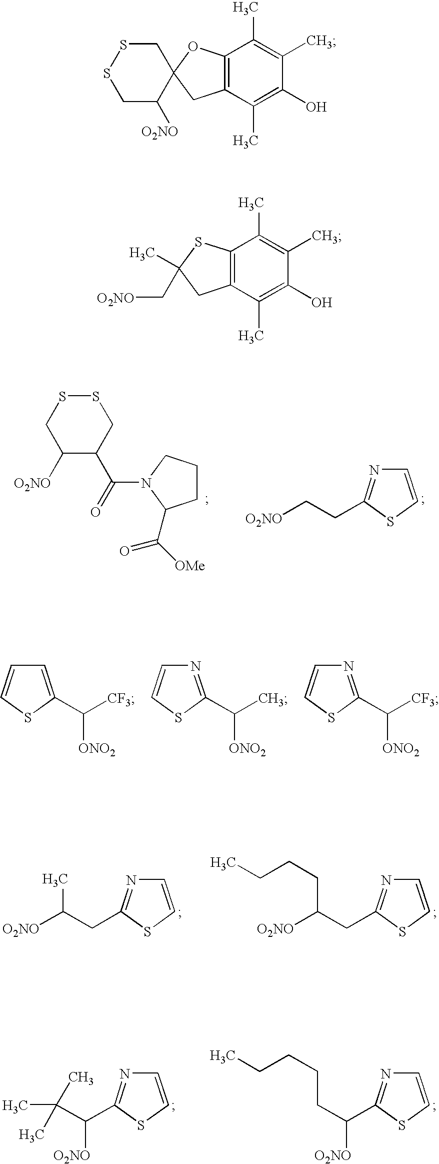

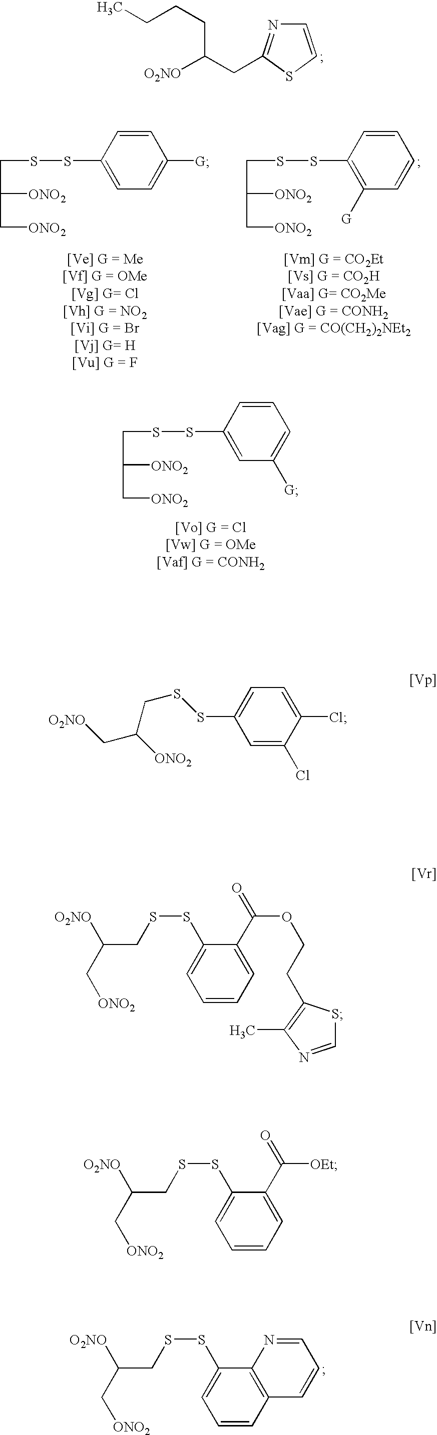

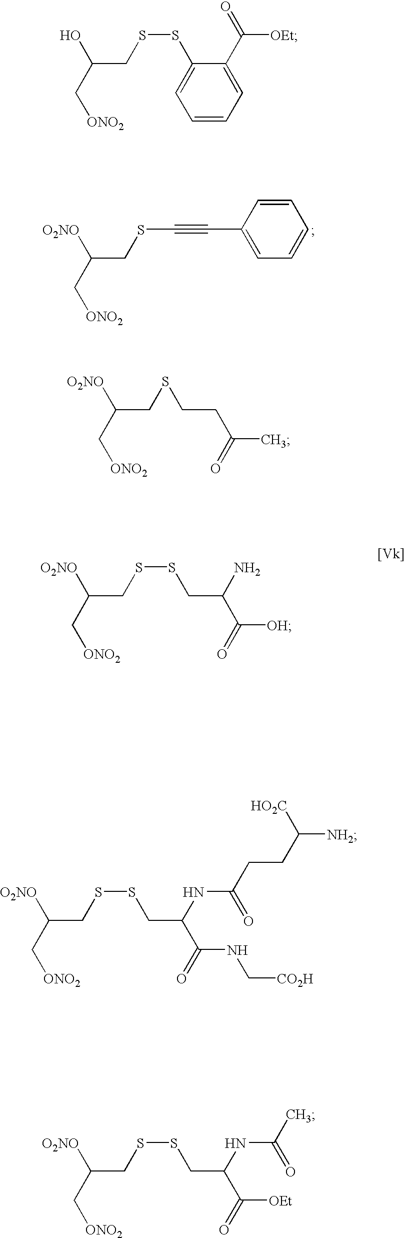

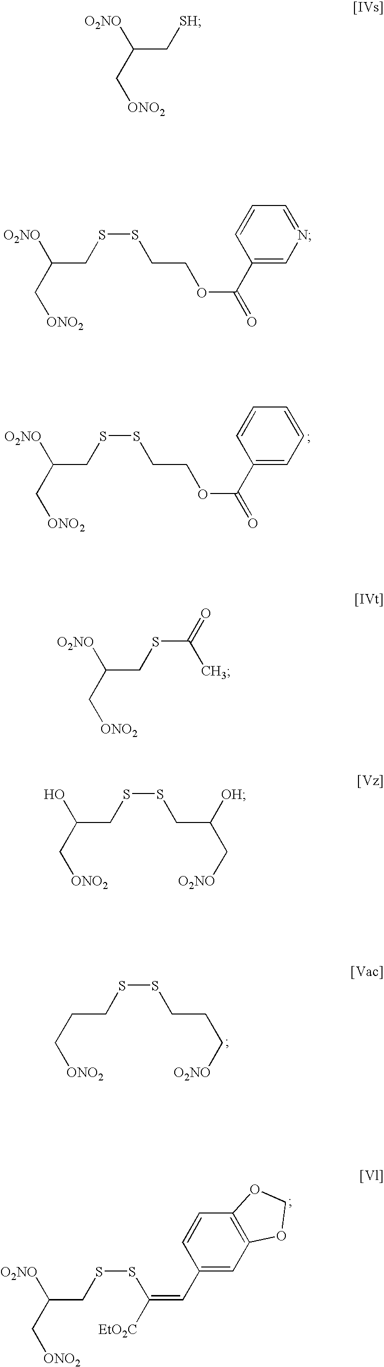

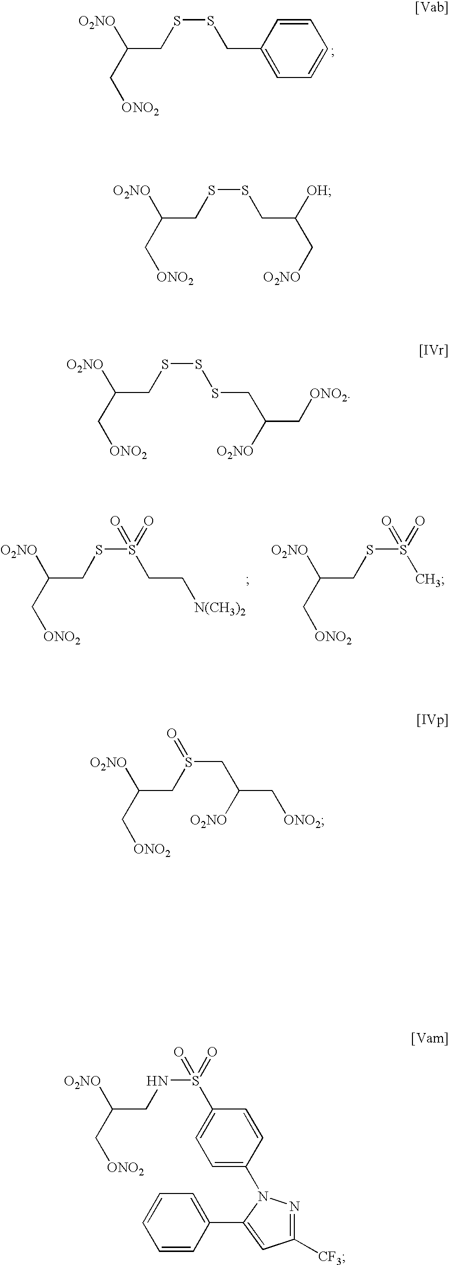

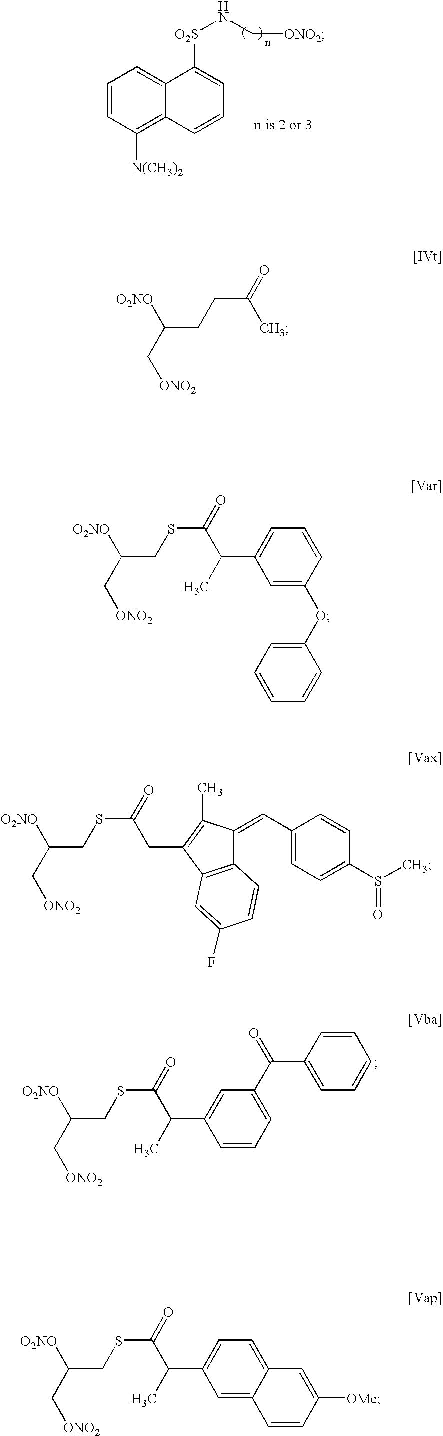

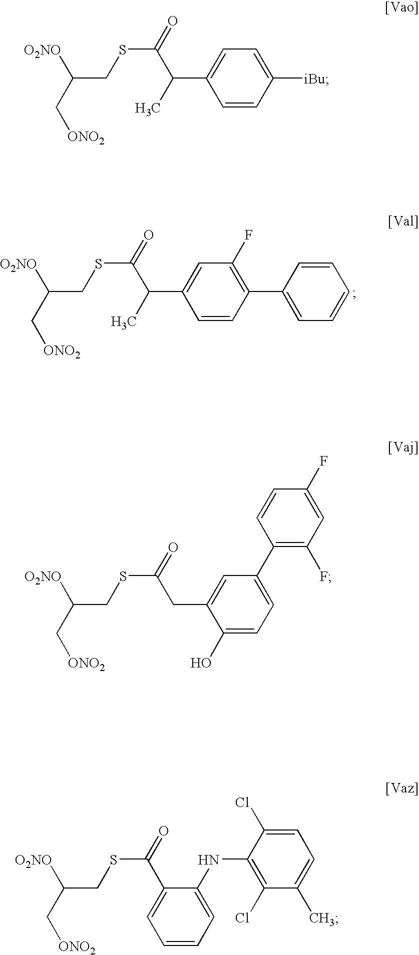

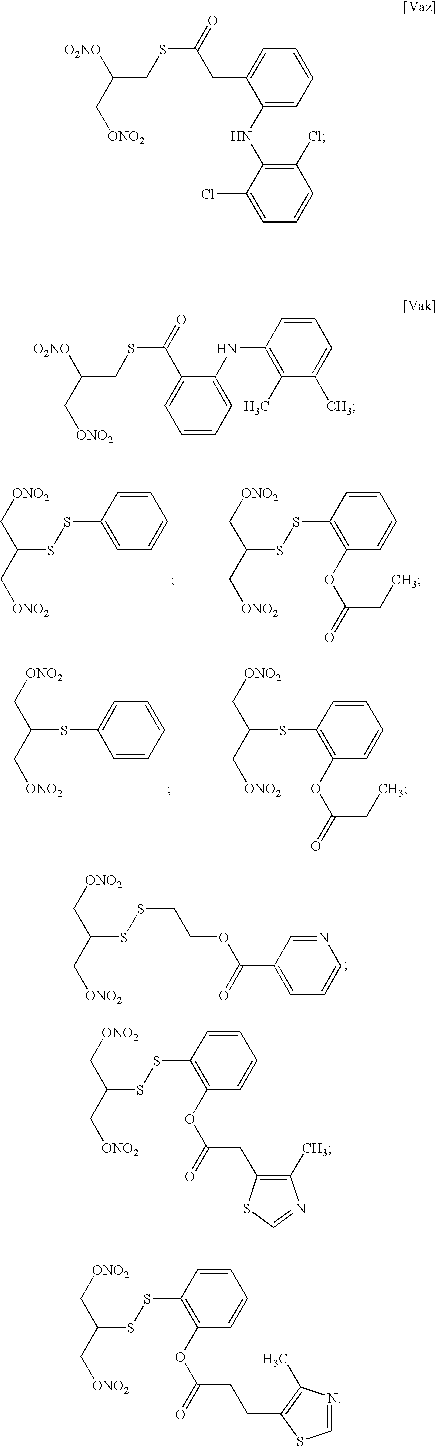

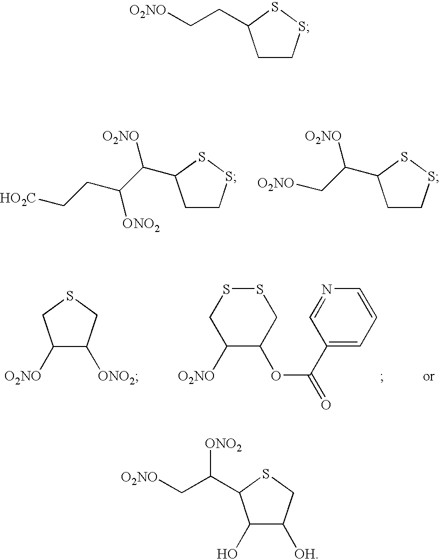

Pharmaceutical compostions comprising a compound of Formula III in admixture with a pharmaceutically acceptable carrier therefor are provided by the invention. The invention further provides methods of mitigating neurodegeneration, effecting neuroprotection and/or effecting cognition enhancement in a subject comprising the step of administering a compound of Formula III to a subject such that said mitigation and/or said neuroprotection and/or cognition enhancement occurs.

In another aspect of the invention, a therapeutic compound of the invention can be represented by the formula (Formula IV): 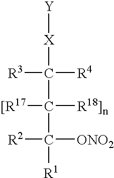
in which n=0, X is CH₂ or does not exist, and Y is selected from F, Br, Cl, CH₃, CF₂H, CF₃, OH, NH₂, NHR₆, NR₆R₇, CN, NHOH, N₂H₃, N₂H₂R₁₃, N₂HR₁₃R₁₄, N₃, S, SCN, SC(═NH)N(R¹⁵)₂, SC(═NH)NHR¹⁵, SC(O)N(R₁₅)₂, SC(O)NHR₁₅, SO₃M, SH, SR₇, SO₂M, S(O)R₈, S(O)₂R₉, S(O)OR₈, S(O)₂OR₉, PO₂HM, PO₃M₂, P(O)(OR₁₅)(OR₁₆), P(O)(OR₁₆)(OM), P(O)(R₁₅)(OR₈), P(O)(OM)R₁₅, CO₂M, CO₂H, CO₂R₁₁, C(O)R₁₂, C(O)(OR₁₃), C(O)(SR₁₃), SR₅, SSR₇ or SSR₅, R₂, R₄, R₅, R₆, R₇, R₈, R₉, R₁₁, R₁₂, R₁₃, R₁₄, R₁₅, and R₁₆ are as defined above. In certain preferred embodiments, R₂ and R₄ are optionally H, a nitrate group or a connection to R₅-R₉ and R₁₁-R₁₆ in cyclic derivatives.

Pharmaceutical compositions comprising a compound of Formula IV in admixture with a pharmaceutically acceptable carrier therefor are provided by the invention.

The invention further provides methods of mitigating neurodegeneration, effecting neuroprotection and/or effecting cognition enhancement in a subject comprising the step of administering a compound of Formula IV to a subject such that said mitigation and/or said neuroprotection and/or cognition enhancement occurs.

Examples and preferred embodiments of compounds of the invention according to Formula IV are as follows:

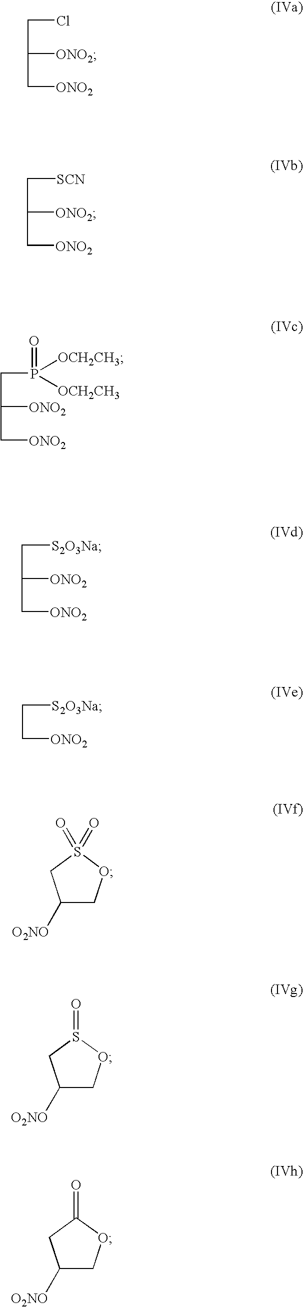

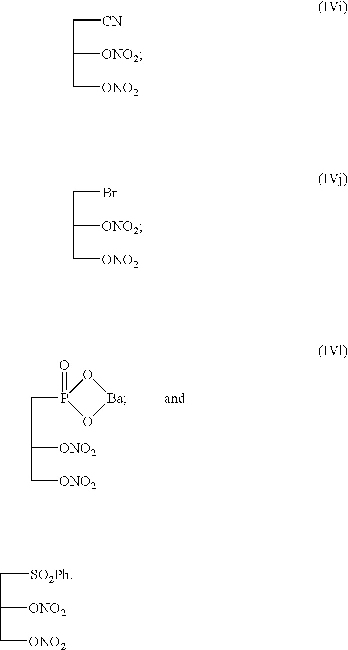

In yet another aspect of the invention, a compound of the invention can be represented by the formula (Formula V): 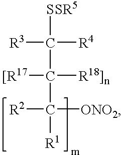
in which R₂ is optionally H or a connection to R₅ in cyclic derivatives, R₄ is H or a nitrate group, and R₅ is as described above.

Pharmaceutical compositions comprising a compound of Formula V in admixture with a pharmaceutically acceptable carrier therefor are provided by the invention.

The invention further provides methods of mitigating neurodegeneration, effecting neuroprotection and/or effecting cognition enhancement in a subject comprising the step of administering a compound of Formula V to a subject such that said mitigation and/or said neuroprotection and/or cognition enhancement occurs.

Examples and preferred embodiments of compounds of the invention according to formula V (Formulae Va-c) are as follows:

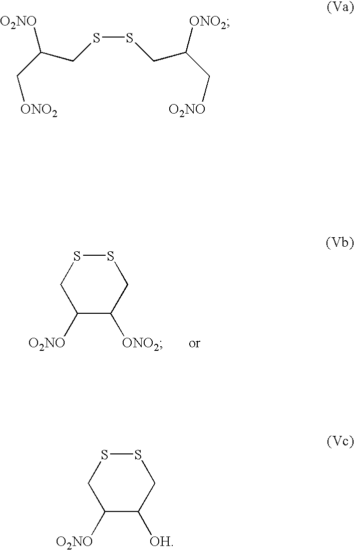

In another aspect, the invention features one of the following nitrate esters:

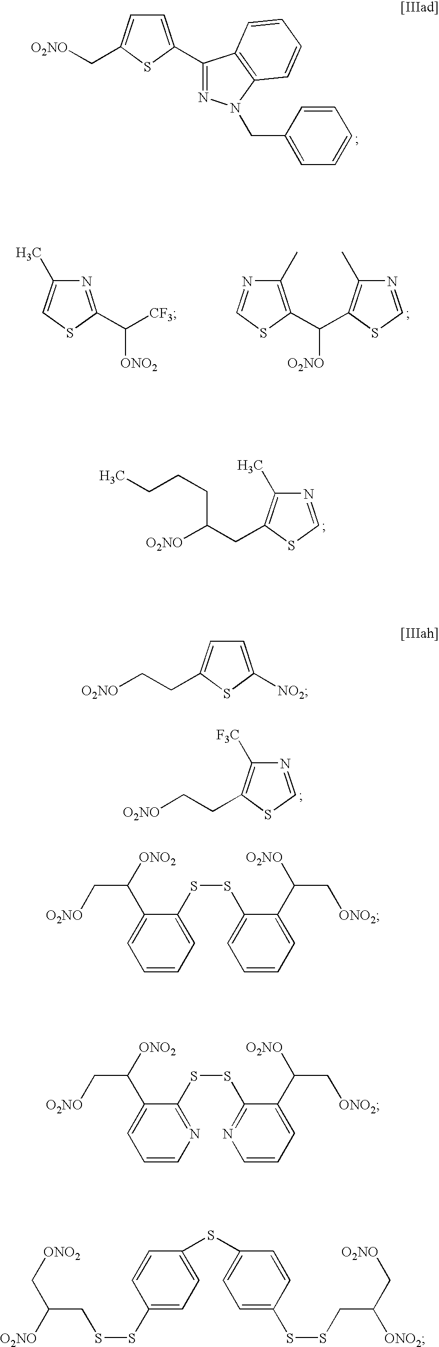

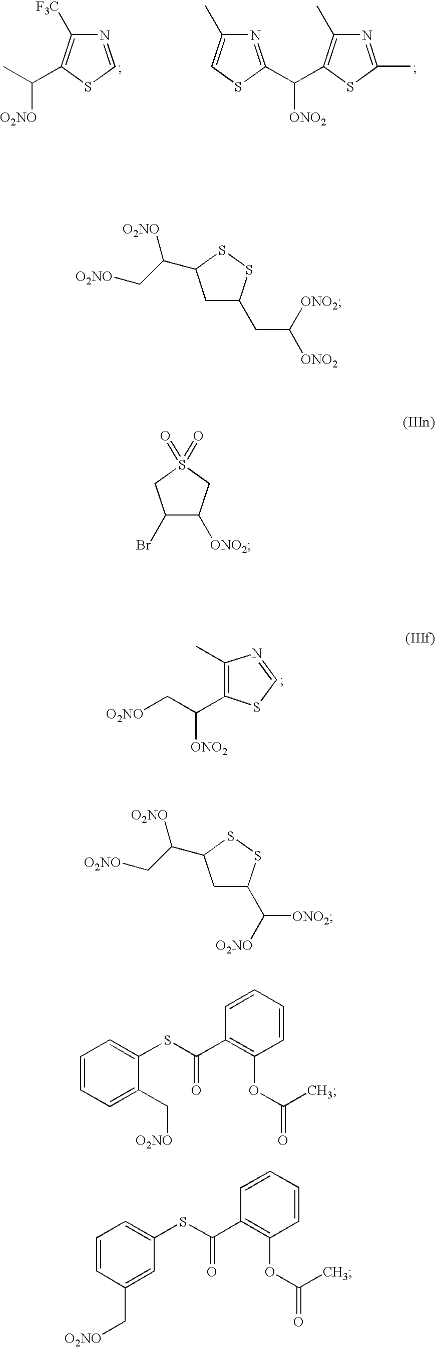

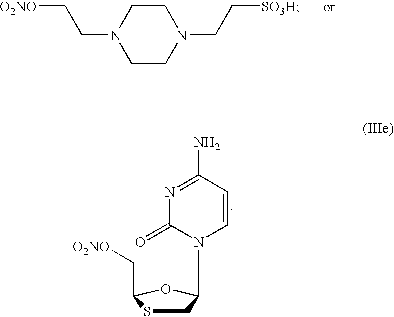

In another aspect, the invention features a method for preventing or mitigating tissue and/or cellular damage in a subject by modulating intercellular and/or intracellular free radical concentration in the subject. The method includes administering to the subject an effective amount of a compound containing at least one aliphatic nitrate group and at least one sulfur atom in proximity to said nitrate, such as, for example, a compound of formula I. In one embodiment, the nitrate of this method contains at least 2 nitrate groups. In another embodiment, the nitrate is beta or gamma to a sulfur atom.

Yet another embodiment features compounds of formula III for preventing or mitigating tissue and/or cellular damage where m is an integer from 0 to 10; n is an integer from 0 to 10; each of R^{3,4,17} is, independently, hydrogen, a nitrate group, or A; R¹ is hydrogen or A, with A is selected from: a substituted or unsubstituted C₁-C₂₄ alkyl group, optionally containing 1 to 4 O, S, NR⁶, and/or unsaturations in the chain, optionally bearing from 1 to 4 hydroxy, nitrate, Cl, F, amino or unsubstituted or substituted aryl, or unsubstituted or substituted heterocyclic groups; an unsubstituted or substituted cyclic moiety having from 3 to 7 carbon atoms in the ring, which optionally contains O, S, NR⁶, and/or unsaturations in the ring, optionally bearing from 1 to 4 hydroxy, nitrate, Cl, F, amino or unsubstituted or substituted aryl, or unsubstituted or substituted heterocyclic groups; an unsubstituted or substituted moiety constituting a linkage from 0 to 5 carbons, to or between any of R¹, R³, R¹⁷ and R⁴, which optionally contains O, S, NR⁶, and/or unsaturations in the linkage, and optionally bearing from 1 to 4 hydroxy, nitrate, Cl, F, amino or unsubstituted or substituted aryl, or unsubstituted or substituted heterocyclic groups; a substituted or unsubstituted C₁-C₂₄ alkyl group, containing 1-4 linkages selected from C═O, C═S, and C═NOH, which optionally contains O, S, NR⁶, and/or unsaturations in the chain, optionally bearing from 1 to 4 hydroxy, nitrate, Cl, F, amino or unsubstituted or substituted aryl, or unsubstituted or substituted heterocyclic groups; a substituted or unsubstituted aryl group; a substituted or unsubstituted heterocyclic group; an amino group selected from alkylamino, dialkylamino, cyclic amino, cyclic diamino, cyclic triamino, arylamino, diarylamino, and alkyarylamino; a hydroxy group; an alkoxy group; and a substituted or unsubstituted aryloxy group;
R², R⁵, R¹⁸, are optionally hydrogen, A, or X-Y;
X is F, Br, Cl, NO₂, CH₂, CF₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)R⁵, S(O)₂R⁵, S(O)OR⁸, S(O)₂OR⁹, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂H, PO₂M, P(O)(OR¹⁴), P(O)(R¹³), SO, SO₂, C(O)(SR¹³), SR⁵, SSR⁷ or SSR⁵, SS, or does not exist;
Y is F, Br, Cl, CH₃, CF₂H, CF₃, OH, NH₂, NHR⁶, NR⁶R⁷, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)R⁵, S(O)₂R⁵, S(O)₂OR⁹, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R⁵, C(O)R¹², C(O)(OR¹³), C(O)(SR¹³), SR⁵, SSR⁷ or SSR⁵, C(S)R⁵, C(S)R¹², C(S)OR¹², or does not exist;
each of R⁶, R⁷, R⁸, R⁹, R¹⁰, R¹¹, R², R¹³, R¹⁴, R¹⁵, and R¹⁶ is, independently, a C₁-C₂₄ alkyl group, optionally containing 1-4 ONO₂ substituents, a C₁-C₂₄ acyl group, optionally containing 1-4 ONO₂ substituents, a C₁-C₆ ring-forming connection to any of R¹-R⁴, a hydrogen, a nitrate group, or A; and
M is H, Na⁺, K⁺, NH₄⁺, N⁺HₖR¹¹₍₄₋ₖ₎ where k is 0-3, or other pharmaceutically acceptable counterion;
and with the proviso that, when m=0 and n=1, each of R¹⁸ and R³ is, individually, H, a nitrate group, or a C₁-C₄ alkyl group, which may include one O, linking R¹⁸ and R³ to form pentosyl, hexosyl, cyclopentyl, or cyclohexyl rings, which optionally bears hydroxyl substituents; each of R¹⁷ and R⁴ is, individually, H, a nitrate group, a C₁-C₄ alkyl group, optionally bearing from 1-3 nitrate groups, or —C(O)R⁵; each of R⁵, R⁶, R⁸, R⁹, R¹², R¹³, R¹⁴, R¹⁵, R¹⁶ is, individually, a C₁-C₂ alkyl group, optionally containing 1-4 ONO₂ substituents or a C₁-C₂ ring-forming connections to R¹⁸, R¹⁷, or R³; each of R⁷ and R¹¹ is, independently, a C₁-C₈ alkyl group or a C₁-C₈ acyl group; M is H, Na⁺, K⁺, NH₄⁺, N⁺HₖR¹¹₍₄₋ₖ₎ where k is 0-3;
X is CH₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸; S(O)₂R⁹, S(O)OR⁸, S(O)₂OR⁹, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂M, P(O)(OR¹⁴), P(O)(R¹³), SO, SO₂, C(O)(SR¹³), SR⁵, or SSR⁴; and
Y is not CN, N₂H₂R¹³, N₂HR¹³R¹⁴, N₃, SCN, SC(═NH)N(R¹⁵)₂ , SC(═NH)NHR¹⁵, SC(O)N(R¹⁵)₂, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R⁵, C(O)R¹², C(O)(SR¹³), SR⁴, SR⁵, or SSR⁵, or Y does not exist.

Preferred compounds include any of the compounds of formulas II, III, IV, and V that have been individually described herein. Other preferred compounds include:

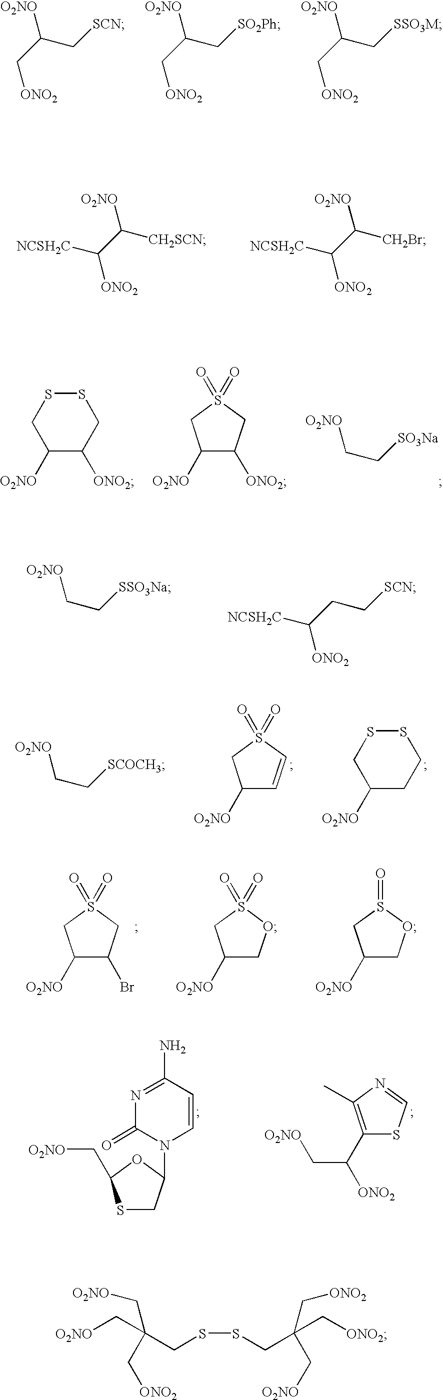

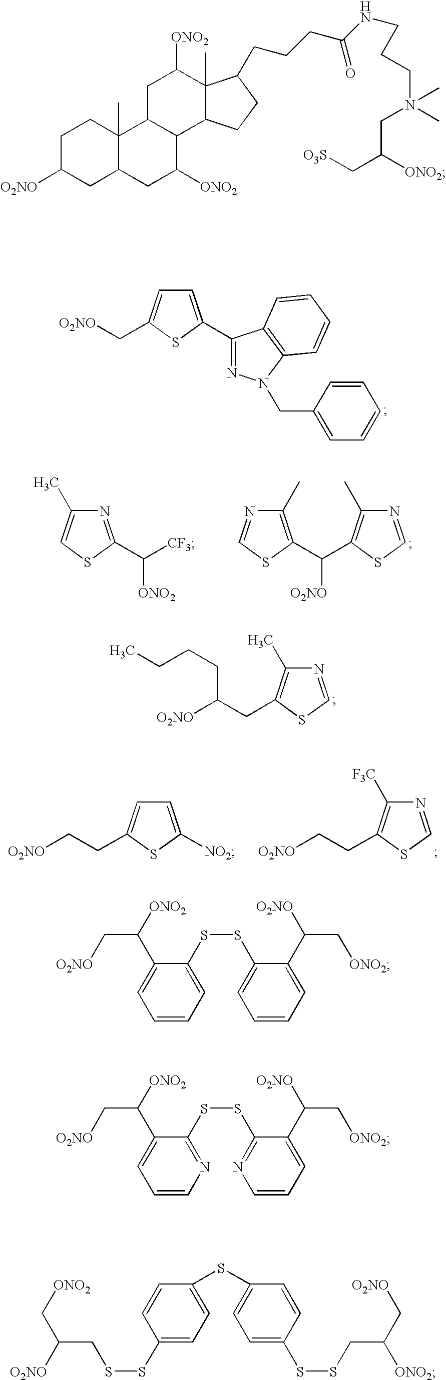

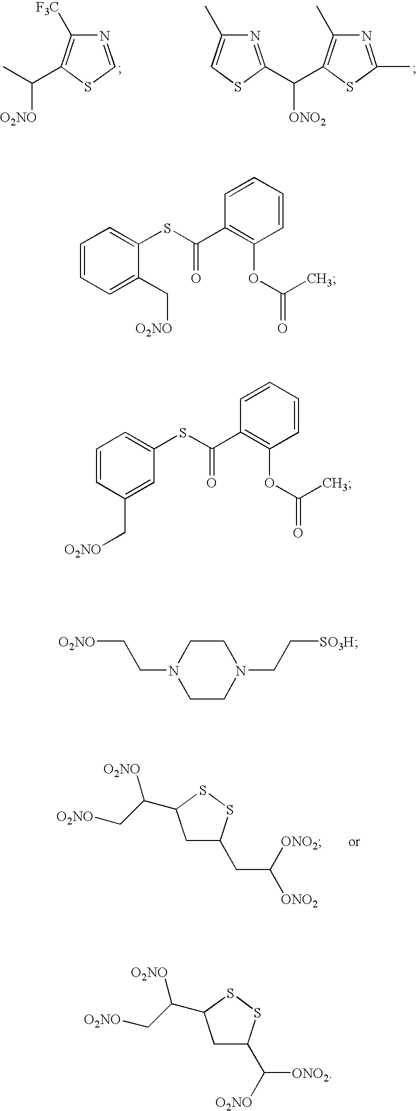

Particularly preferred compounds for preventing or mitigating tissue and/or cellular damage are:

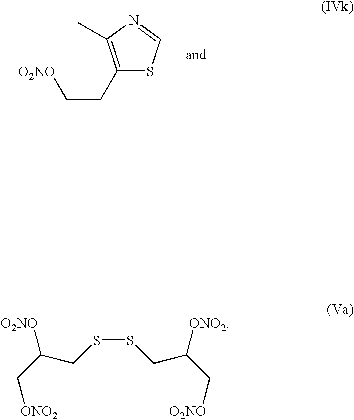

It will be noted that the structure of some of the compounds of this invention includes asymmetric carbon atoms. It is to be understood accordingly that the isomers (e.g., enantiomers, diastereomers) arising from such asymmetry are included within the scope of this invention. Such isomers can be obtained in substantially pure form by classical separation techniques and by asymmetric synthesis. For the purposes of this application, unless expressly noted to the contrary, a compound shall be construed to include both the R and S stereoisomers at each stereogenic center.

In certain embodiments, a therapeutic compound of the invention comprises a cation (i.e., in certain embodiments, one of X or Y includes a cation, e.g., in the compound of formula IVd). If the cationic group is a proton, then the compound is considered an acid. If the proton is replaced by a metal ion or its equivalent, the compound is a salt. Pharmaceutically acceptable salts of the therapeutic compound are within the scope of the invention. For example, M can be a pharmaceutically acceptable alkali metal (e.g. Li, Na, K), ammonium, alkaline earth metal (e.g. Ca, Ba, Mg), higher valency cation, or polycationic counter ion (e.g., polyammonium cation) (see e.g., Berge et al., 1977). It will be appreciated that the stoichiometry of an anionic portion of the compound to a salt-forming cation will vary depending on the charge of the anionic portion of the compound and the charge of the counterion. Preferred pharmaceutically acceptable salts include a sodium, potassium, or calcium salt, but other salts are also contemplated within their pharmaceutically acceptable range.

A therapeutic compound of the invention can be administered in a pharmaceutically acceptable vehicle. As used herein “pharmaceutically acceptable vehicle” includes any and all solvents, excipients, dispersion media, coatings, antibacterial and antifungal agents, isotonic and absorption delaying agents, and the like which are compatible with the activity of the compound and are physiologically acceptable to the subject. An example of a pharmaceutically acceptable vehicle is buffered normal saline (0.15 M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

Carrier or substituent moieties useful in the present invention may also include moieties which allow the therapeutic compound to be selectively delivered to a target organ. For example, delivery of the therapeutic compound to the brain may be enhanced by a carrier moiety using either active or passive transport (a “targeting moiety”). Illustratively, the carrier molecule may be a redox moiety, as described in, for example, U.S. Pat. Nos. 4,540,654 and 5,389,623, both to Bodor. These patents disclose drugs linked to dihydropyridine moieties which can enter the brain, where they are oxidized to a charged pyridinium species which is trapped in the brain. Thus drugs accumulate in the brain. Other carrier moieties include compounds, such as amino acids or thyroxine, which can be passively or actively transported in vivo. Such a carrier moiety can be metabolically removed in vivo, or can remain intact as part of an active compound. Structural mimics of amino acids (and other actively transported moieties) including peptidomimetics, are also useful in the invention. As used herein, the term “peptidomimetic” is intended to include peptide analogues which serve as appropriate substitutes for peptides in interactions with, for example, receptors and enzymes. The peptodomimetic must possess not only affinity, but also efficacy and substrate function. That is, a peptidomimetic exhibits functions of a peptide, without restriction of structure to amino acid constituents. Peptidomimetics and methods for their preparation and use are described in Morgan et al. (1989), the contents of which are incorporated herein by reference. Many targeting moieties are known, and include, for example, asialoglycoproteins (see e.g., Wu, U.S. Pat. No. 5,166,320) and other ligands which are transported into cells via receptor-mediated endocytosis (see below for further examples of targeting moieties which may be covalently or non-covalently bound to a target molecule).

In the methods of the invention, neurodegeneration in a subject is mitigated, and/or neuroprotection and/or cognition enhancement is effected, by administering a therapeutic compound of the invention to the subject. The invention also features methods for preventing or mitigating tissue and/or cellular damage in a subject by administering a therapeutic compound of the invention to the subject, thereby modulating intercellular and/or intracellular free radical concentration. The term “subject” is intended to include living organisms in which the particular neurological condition to be treated can occur. Examples of subjects include humans, apes, monkeys, cows, sheep, goats, dogs, cats, mice, rats, and transgenic species thereof. As would be apparent to a person of skill in the art, the animal subjects employed in the working examples set forth below are reasonable models for human subjects with respect to the tissues and biochemical pathways in question, and consequently the methods, therapeutic compounds and pharmaceutical compositions directed to same. As evidenced by Mordenti (1986) and similar articles, dosage forms for animals such as, for example, rats can be and are widely used directly to establish dosage levels in therapeutic applications in higher mammals, including humans.

In particular, the biochemical cascade initiated by cerebral ischemia is generally accepted to be identical in mammalian species (Mattson and Scheff, 1994; Higashi et al., 1995). In light of this, pharmacological agents that are neuroprotective in animal models such as those described herein are believed to be predictive of clinical efficacy in humans, after appropriate adjustment of dosage. Specifically, there are comparable memory-deficit patterns between brain-damaged rats and humans, which indicates that the rat can serve as an excellent animal model to evaluate the efficacy of pharmacological treatments or brain damage upon memory (Kesner, 1990). The only approved drug for the clinical treatment of occlusive stroke in humans is tissue plasminogen activator, which is administered at a dose of 0.9 mg/kg by intravenous injection (Wittkowsky, 1998). This drug is also effective in protecting the rat brain subjected to cerebral ischemia by occlusion of the middle cerebral artery, when administered at a dose of 10 mg/kg intravenously (Jiang et al., 1998). Thus, the rat model of focal cerebral ischemia used in the development of the novel organic nitrate esters described herein has been shown to be shown to be predictive of clinical efficacy with at least one other class of pharmacological agents.

As would also be apparent to a person skilled in the art, the invention further encompasses methods of the invention employed ex vivo or in vitro. For example, the Examples describe studies utilizing tissue homogenates according to the invention. Furthermore, diagnostic tests or studies of efficacy of selected compounds may conveniently be performed ex vivo or in vitro, including in animal models. Such tests, studies and assays are within the scope of the invention.

The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000, Philadelphia, and Encyclopedia of Pharmaceutical Technology, eds. J. Swarbrick and J. C. Boylan, 1988-1999, Marcel Dekker, New York). Administration of the compositions of the present invention to a subject to be treated can be carried out using known procedures, at dosages and for periods of time effective to mitigate neurodegeneration, to effect neuroprotection, to effect cognition enhancement, and/or to prevent or mitigate tissue and/or cellular damage in the subject. An effective amount of the therapeutic compound necessary to achieve a therapeutic effect may vary according to factors such as the amount of neurodegeneration that has already occurred at the clinical site in the subject, the age, sex, and weight of the subject, and the ability of the therapeutic compound to mitigate neurodegeneration, to effect neuroprotection, to effect cognition enhancement, and/or to prevent or mitigate tissue and/or cellular damage in the subject. Dosage regimens can be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention (e.g., Va) is between 0.5 and 500 mg/kg of body weight per day. In an aqueous composition, preferred concentrations for the active compound (i.e., the therapeutic compound that can mitigate neurodegeneration, effect neuroprotection, effect cognition enhancement, and/or prevent or mitigate tissue and/or cellular damage) are between 5 and 500 mM, more preferably between 10 and 100 mM, and still more preferably between 20 and 50 mM.

The therapeutic compounds of the invention can be effective when administered orally. Accordingly, a preferred route of administration is oral administration. Alternatively, the active compound may be administered by other suitable routes such as transdermal, subcutaneous, intraocular, intravenous, intramuscular or intraperitoneal administration, and the like (e.g., by injection). Depending on the route of administration, the active compound may be coated in a material to protect the compound from the action of acids, enzymes and other natural conditions which may inactivate the compound.

The compounds of the invention can be formulated to ensure proper distribution in vivo. For example, the blood-brain barrier (BBB) excludes many highly hydrophilic compounds. To ensure that the therapeutic compounds of the invention cross the BBB, they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., Ranade et al., 1989). Exemplary targeting moieties include folate and biotin (see, e.g., U.S. Pat. No. 5,416,016 to Low et al.); mannosides (Umezawa et al., 1988); antibodies (Bloeman et al., 1995; Owais et al., 1995); and surfactant protein A receptor (Briscoe et al., 1995). In a preferred embodiment, the therapeutic compounds of the invention are formulated in liposomes; in a more preferred embodiment, the liposomes include a targeting moiety.

Delivery and in vivo distribution can also be affected by alteration of an anionic group of compounds of the invention. For example, anionic groups such as phosphonate or carboxylate can be esterified to provide compounds with desirable pharmocokinetic, pharmacodynamic, biodistributive, or other properties. Exemplary compounds include IVl and pharmaceutically acceptable salts or esters thereof.

To administer the therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984).

The therapeutic compound may also be administered parenterally (e.g., intramuscularly, intravenously, intraperitoneally, intraspinally, or intracerebrally). Dispersions can be prepared in glycerol, liquid polyethylene glycols, and mixtures thereof and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and vegetable oils. The proper fluidity can be maintained, for example, by the use of a coating such as lecithin, by the maintenance of the required particle size in the case of dispersion, and by the use of surfactants.

Prevention of the action of microorganisms can be achieved by various antibacterial and antifungal agents, for example, parabens, chlorobutanol, phenol, ascorbic acid, thimerosal, and the like. In some cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of neurological conditions in subjects.

Therapeutic compositions can be administered in time-release or depot form, to obtain sustained release of the therapeutic compounds over time. The therapeutic compounds of the invention can also be administered transdermally (e.g., by providing the therapeutic compound, with a suitable carrier, in patch form).

Active compounds are administered at a therapeutically effective dosage sufficient to mitigate neurodegeneration, to effect neuroprotection, to effect cognition enhancement, and/or to prevent or mitigate tissue and/or cellular damage in a subject. A “therapeutically effective dosage” preferably mitigates neurodegeneration by about 20%, more preferably by about 40%, even more preferably by about 60%, and still more preferably by about 80% relative to untreated subjects. The ability of a compound to mitigate neurodegeneration can be evaluated in model systems that may be predictive of efficacy in mitigating neurodegeneration in human diseases, such as animal model systems known in the art (including, e.g., the method of transient middle cerebral artery occlusion in the rat) or by in vitro methods, (including, e.g., the assays described herein).

It will be appreciated that the ability of a compound of the invention to mitigate neurodegeneration will, in certain embodiments, be evaluated by observation of one or more symptoms or signs associated with neurodegeneration in vivo. Thus, for example, the ability of a compound to mitigate neurodegeneration may be associated with an observable improvement in a clinical manifestation of the underlying neurodegeneration-related disease state or condition, or a slowing or delay in progression of symptoms of the condition. Thus, monitoring of clinical manifestations of disease can be useful in evaluating the neurodegeneration-mitigating efficacy of a compound of the invention.

The method of the invention is useful for treating neurodegeneration associated with any disease in which neurodegeneration occurs. Clinically, neurodegeneration can be associated with conditions including but not limited to: stroke; Parkinson's disease; Alzheimer's disease; Huntington's disease; multiple sclerosis; amylotrophic lateral sclerosis; AIDS-induced dementia; epilepsy; alcoholism; alcohol withdrawal; drug-induced seizures; viral/bacterial/fever-induced seizures; trauma to the head; hypoglycemia; hypoxia; myocardial infarction; cerebral vascular occlusion; cerebral vascular hemorrhage; hemorrhage; environmental excitotoxins of plant; animal, or marine origin; dementias of all type; trauma; drug-induced brain damage; and aging; or result from surgical procedures such as cardiac bypass.

Novel compounds according to the invention can be synthesized by methods set forth herein or in U.S. Pat. Nos. 5,807,847; 5,883,122; 6,310,052; and 6,365,579, for example. Various compounds for use in the methods of the invention are commercially available and/or can be synthesized by standard techniques. In general, nitrate esters can be prepared from the corresponding alcohol, oxirane or alkene by standard methods, that include: nitration of alcohols and oxiranes, mixed aqueous/organic solvents using mixtures of nitric and sulfuric acid and/or their salts, with temperature control (see Yang et al., 1996); nitration of alcohols and oxiranes in acetic anhydride using nitric acid or its salts with or without added acid catalyst, with temperature control (see, e.g., Louw et al., 1976); nitration of an alcohol with a nitronium salt, e.g. a tetrafluoroborate; nitration of an alkene with thallium nitrate in an appropriate solvent (see Ouellette et al., 1976).

The following Examples further illustrate the present invention and are not intended to be limiting in any respect. Those skilled in the art will recognize, or be able to ascertain using no more than routine experimentation, numerous equivalents to the specific procedures described herein. Such equivalents are considered to be within the scope of this invention and are covered by the claims.

### EXAMPLE 1

### Preparation of Nitrate Esters

Synthesis of Compound IVr

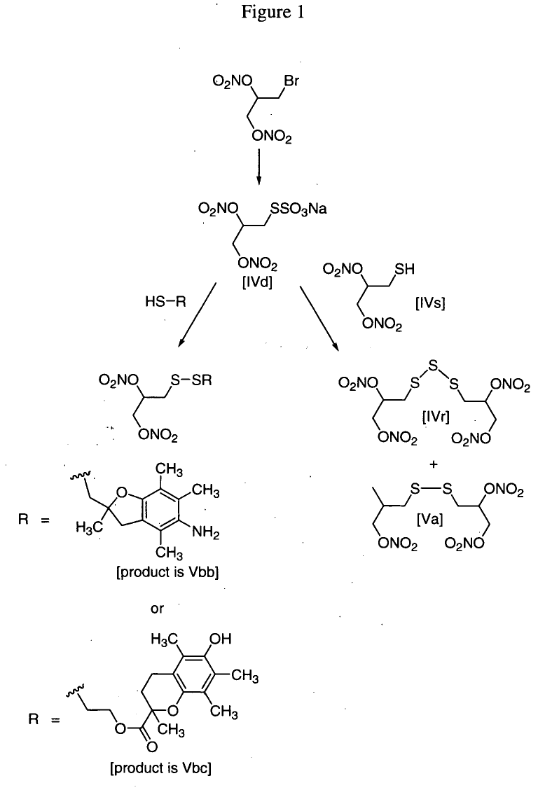

As shown in FIG. 1, the synthesis of compound IVr proceeded from the Bunte salt, 2,3-dinitrooxypropane-1-thiosulfonate (compound IVd), which was prepared from 1,2-dinitrooxy-3-bromopropane as follows: 3-bromopropane-1,2-diol was added dropwise into a cold mixture of HNO₃ (68-70%, 4.0eq) and H₂SO₄ (95%, 4.0 eq) in CH₂Cl₂ (50 mL) at room temperature over 30 min. The organic layer was separated, washed, dried and concentrated to yield a yellow oil which was purified by flash chromatography on SiO₂ to give 3-bromopropane-1,2-diol dinitrate in 45% yield (29). The Bunte salt was prepared by reacting 3-bromopropane-1,2-diol dinitrate with an equimolar portion of Na₂S₂O₃ in 3:1 MeOH/H₂O at 50° C. for 10 hours and subsequently purifying by flash chromatography on SiO₂ (29). The Bunte salt was oxidized with a small molar excess of H₂O₂ (30%) in EtOH:H₂O mixture (1:1) with a catalytic amount of H₂SO₄ for 2 days. Extraction with CH₂Cl₂, concentration, and purification by flash chromatography on SiO₂ yielded compound IVr as a yellow oil (R_{f}=0.65; CH₂Cl₂:hexane=65:35; 5%). ¹H-NMR (CDCl₃, 400 MHz): 5.55-5.65 (m, 1H), 4.87-4.94 (dd, 1H, J 12.94, 2.94), 4.62-4.70 (m, 1H, J 12.88), 3.13-3.30 (m, 2H). ¹³C-NMR: (CDCl₃, 100 MHz): 76.74, 69.46/69.42, 36.65/36.63. Mass spec. (m/z, EI⁺): 380.1 (M-NO₂)⁺ 290%; 426.1 (M)⁺ 100%; 427.1 (M+1)⁺ 10%; 428.1 (M+2)⁺ 17%; 429.2 (M+3)⁺ 1.5%; 430.3 (M+4)⁺ 1.3%; calculated for C₆H₁₀N₄O₁₂S₃ 426.0. Elemental analysis: calculated for C₆H₁₀N₄O₁₄S₂: C, 16.90; H, 2.36; S, 15.04; calculated for C₆H₁₀N₄O₁₂S₃: C, 16.90; H, 2.36; S, 22.56; found: C, 17.27; H, 2.38; S, 21.68.

Synthesis of Compound Va, Vbb, and Vbc

As shown in FIG. 1, the syntheses of compounds Va, Vbb, and Vbc proceeded from the Bunte salt, compound IVd. A round-bottomed flask equipped with a dropping funnel, a thermometer, and a mechanical stirrer was charged with a solution of compound IVd and cooled to 0° with the aid of an ice-salt bath. A cold solution of the thiol precursor of compounds Va, Vbb, or Vbc was added rapidly, with vigorous stirring for 3 minutes, followed by the addition of aqueous saturated NaCl. The mixture was warmed to 5° C. and stirring stopped after 10 minutes. The crude disulfides Va, Vbb, or Vbc, were extracted 3× with diethyl ether. The extracts were combined, dried over calcium sulfate, and filtered through a glass-wool plug. Removal of the solvent leaves disulfide product, which can be further purified by silica gel chromatography.

Synthesis of Compounds Ve, Vf, Vg, Vh, Vi, Vj, and Vu

Alkyl bromides or alkyl mercaptans were obtained commercially or by adaptation of literature procedures. Bunte salts were obtained from the appropriate alkyl bromide by reaction with sodium thiosulfate, as described above for compound IVd. Bunte salts (9.67 mmoles) were dissolved in distilled water (10 mL). To this solution, a solution of mercaptan (6.46 mmoles) in 1M NaOH (7 mL) was added dropwise. The resulting emulsion was stirred for 1 to 15 minutes and then extracted with dichlorometane or ethylacetate. The combined organic extracts were washed with H₂O, dried over MgSO₄ and concentrated under vacuum. The residue was purified by flash column chromatography on silica gel to give the following: Compound Vj (53% yield), ¹³C NMR (75 Mhz, CDCl₃) 36.9, 69.8, 77.6, 128.6, 129.5, 129.8, 136.0; Compound Vi (43% yield), ¹³C NMR (75 Mhz, CDCl₃) 36.9, 69.8, 77.4, 122.7, 130.9, 132.9, 135.1; Compound Vh (9% yield), ¹³C NMR (75 Mhz, CDCl₃) 36.9, 69.6, 77.6, 124.5, 126.9, 144.7, 147.0; Compound Vg (81% yield), ¹³C NMR (75 Mhz, CDCl₃) 36.8, 69.8, 77.5, 129.9, 130.8, 134.5, 134.8; Compound Vf (56% yield), ¹³C NMR (75 Mhz, CDCl₃) 36.5, 55.8, 69.7, 77.8, 115.4, 126.7, 133.7, 160.9; Compound Ve (52% yield), ¹³C NMR (75 Mhz, CDCl₃) 21.5, 36.8, 69.8, 77.7, 130.5, 130.6, 132.5, 139.2; and Compound Vuj (61% yield), ¹³C NMR (75 Mhz, CDCl₃) 36.7, 69.9, 77.6, 116.8-117.1(d), 131.3-131.3(d), 132.5-132.6(d), 161.6-164.9(d).

Synthesis of Compound Vb

1,4-dibromo-2,3-dinitrobutanediol (8.88 mmol) and Na₂S₂O₃.5H₂O (2.81 g; 18 mmol) were dissolved in a mixture of 100 mL of MeOH and 45 mL of H₂O. The resulting solution was heated during 4 days at 40-45°. After this time the reaction mixture was partially evaporated to reduce the volume of solvents. The resulting mixture was extracted with 4×50 mL of Et₂O. The extracts were combined, washed (H₂O), dried (MgSO₄) and concentrated. The residue was purified by flash column chromatography on silica gel (hexane:EtOAc 85:15), yielding the title compound (10%) ¹³C(CDCl₃): δ3.9, 31.7.

Tables 1 and 2 list characterization data determined for other compounds of the invention.

TABLE 1

| | ¹H NMR | ¹³C NMR |
|---|---|---|
| IIIa | (CDCl₃): 5.34-5.57(1H, dm, ³J_{HF} 20.6), 4.53-4.87 | (CDCl₃): 79.47(d, ¹J_{CF} 177), 76.73 |
| | (4H, superposition several multiplets, O₂NO—CH₂ + CH₂F, | (d, ²J_{CF} 20.6), 67.84(d, ³J_{CF} 6.87) |
| | ²J_{HF} 46.7, ⁴J_{HF} 0.66) | |
| IIIb | (CDCl₃): δ | (CDCl₃): δ |
| IIIc | (CDCl₃): δ 5.7(1H, t, ²J_{HF} 54), 5.45(1H, m), 4.5-4.9 | (CDCl₃): δ 75.55, 68.05, 60.76 |
| | (2H, m), 4.15-4.35(1H, m) | |
| IIId | (CDCl₃): δ 5.46(1H, m), 4.80-4.87(1H, dd, J 3.5, | (CDCl₃): δ 77.24, 68.57, 39.86 |
| | 12.9), 4.65-4.72(1H, dd, J 6.2, 12.9), 3.7-3.8(2H, | |
| | m) | |
| IIIf | (CDCl₃) δ 8.72(s, 1H), 5.38(t, 1H), 4.6(d, 2H), | — |
| | 2.45(s, 3H) | |
| IIIg | (DMSOd₆) CHONO₂ only: δ 4.8-5.8 | (DMSOd₆) CONO₂ only: δ 85.68, |
| | | 84.17, 82.47, 76.50 |
| IIIh | (CD₃OD) δ 4.85(3H, m), 3.5(1H, m) | (CD₃OD) δ 70.61, 36.74 |
| IIIi | (CDCl₃): δ 6.95(dd, 1H), 6.71(dd, 1H), 6.09(m, | (CDCl₃): δ 137.9, 132.5, 76.6, 52.9 |
| | 1H), 3.80(dd, 1H), 3.32(dd, 1H) | |
| IIIj | (CDCl₃): δ 5.62(2H, m), 3.60(4H, m) | (CDCl₃): δ 77.87, 25.22 |
| IIIk | (CD₃CN): δ 3.45(m, 2H), 5.72(m, 2H) | (CD₃CN): δ 79.98, 28.87 |
| IIIl | — | (CD₃CN): δ 79.48, 33.45, 28.47 |
| IIIm | (DMSOd₆): δ 5.97(m, 2H), 3.80(m, 4H) | (DMSOd₆): δ 78.84, 52.60 |
| IIIn | (CDCl₃): δ 5.73(m, 1H), 4.62(m, 1H), 3.96-3.77 | (CDCl₃): δ 81.47, 57.85, 53.50, |
| | (m, 1H), 3.58-3.32(m, 1H) | 38.75 |
| IIIo | — | (CDCl₃): δ 81.24, 69.79, 33.26, |
| | | 27.24 |
| IIIp | (CDCl₃): δ 5.36(m, 1H), 3.11-3.60(m, 4H), 2.33 | (CDCl₃): δ 78.92, 33.66, 30.64, |
| | (m, 2H) | 27.36 |
| IIIq | (CDCl₃): δ 5.47(m, 1H), 3.53-3.05(m, 4H), 2.29 | (CDCl₃): δ 81.32, 37.12, 32.97, |
| | (m, 2H) | 30.98 |
| IVi | (CDCl₃): δ 5.45(1H, m), 4.83(1H, dd), 4.65(1H, | (CD₃OD): δ 116.44, 75.37, 71.20, |
| | dd), 2.9(2H, m) | 19.19 |
| IVk | (CDCl₃) δ 8.55(s, 1H), 4.55(t, 2H), 3.15(t, 2H), | (CDCl₃) δ 150.9, 150.7, 125.3, |
| | 2.37(s, 3H) | 72.53, 24.47, 15.18 |
| Vb | (CDCl₃) δ 5.56(m, 2H), 3.38-2.95(m, 4H) | (CD₃OD) δ 85.93, 32.77 |
| Vc | (CDCl₃): δ 5.85-5.91(1H, m), 4.50-4.58(1H, m), | (CDCl₃): δ 87.6, 74.96, 36.20, 31.54 |
| | 3.22-3.29(1H, dd, J 5.47, 12.78), 2.97-3.05(1H, | |
| | dd, J 4.6, 11.88), 2.82-2.90(1H, dd, J 2.87, 12.78), | |
| | 2.74-2.83(1H, dd, J 3.15, 11.9) | |

TABLE 2

| | ¹H NMR | ¹³C NMR |
|---|---|---|
| 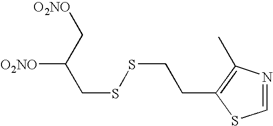 | (CDCl₃): δ 2.43 (s, 3H), 2.89-3.05 (4H, superposition of a triplet with multiplets), 3.18 (t, 2H), 4.8 (dd, 2H), 5.51 (m, 1H), 8.62 (s, 1H) | — |
| 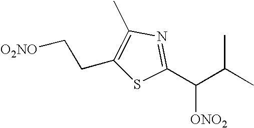 | (CDCl₃): δ 0.95 (d, 3H, J=6.9), 1 (d, 3H, J=6.9), 2.15 (m, 1H), 2.36 (s, 3H), 3.13 (t, 2H, J=6.8), 4.6 (t, 2H, J=6.8), 4.7 (d, 1H, J=5.1). | — |
| 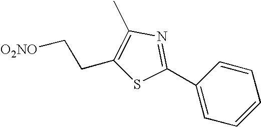 | (CDCl₃): δ 2.44 (s, 3H), 3.21 (t, J=6.7, 2H), 4.63 (t, J=6.7, 2H), 7.4 (m, 3H), 7.9 (m, 2H) | (CDCl₃): δ 15.27, 24.67, 72.37, 125.24, 126.43, 129.05, 130.04, 133.7, 151.1, 166. |
| 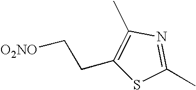 | (CDCl₃): δ 2.33 (s, 3H), 2.63 (s, 3H), 3.11 (t, 2H), 4.57 (t, 2H) | (CDCl₃): δ 163.58, 149.29, 124.36, 72.42, 24.43, 19.26, 14.94 |
| 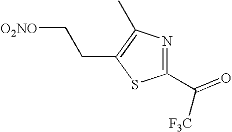 | (CDCl₃): δ 2.57 (s, 3H), 3.32 (t, J=6.43, 2H), 4.68 (t, J=6.43, 2H). | (CDCl₃): δ 15.45, 25.19, 71.31, 114.34, 118.18, 138.18, 155.29, 156.31 |
| 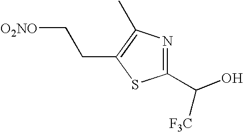 | (CDCl₃): δ 5.21 (q, 1H), 4.62 (t, 2H), 3.2 (t, 2H), 2.4 (s, 3H) | (CDCl₃): δ 160.64, 149.8, 128.3, 125.11, 121.36, 71.9, 24.53, 14.75. |
| 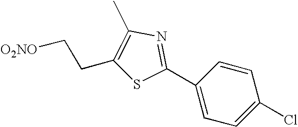 | (CDCl₃): δ 7.82 (d, 2H, J=8.4), 7.4 (d, 2H, J=8.4), 4.64 (t, 2H), 3.21 (t, 2H), 2.43 (s, 3H) | (CDCl₃) 164.13, 151.35, 136.02, 132.24, 129.34, 127.66, 125.71, 72.28, 24.69, 15.25 |
| 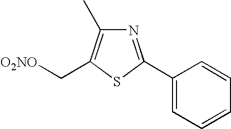 | (CDCl₃): δ 7.91 (m, 2H), 7.44 (m, 3H), 4.61 (s, 2H), 2.54 (s, 3H) | — |
| 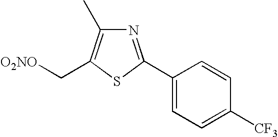 | (CDCl₃): δ 8.03 (d, 2H), 7.7 (d, 2H), 5.62 (t, 2H), 2.56 (s, 3H) | (CDCl₃): δ 166.73, 155.86, 132.5, 132.06, 126.98, 126.25 (q), 122.87, 65.99, 15.56 |
| 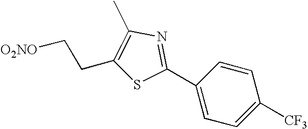 | (CDCl₃): δ 8.0 (d, 2H), 7.68 (d, 2H), 4.65 (t, 2H), 3.23 (t, 2H), 2.46 (s, 3H) | (CDCl₃): δ 163.52, 151.78, 136.8, 126.75, 126.64, 126.13 (q), 125.91, 122.3, 72.19, 24.7, 15.25 |
| 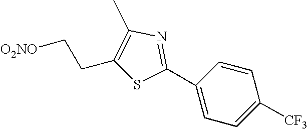 | (CDCl₃): δ 7.78 (d, 2H), 7.23 (d, 2H), 4.63 (t, 2H), 3.19 (t, 2H), 2.39 (s, 3H) | (CDCl₃): δ 165.74, 150.88, 140.36, 131.05, 129.79, 126.4, 124.67, 72.4, 24.65, 21.6, 15.24 |
| 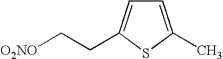 | (CDCl₃): δ 6.62 (dd, 1H), 6.56 (dd, 1H), 4.56 (t, 2H), 3.09 (t, 2H), 2.49 (s, 3H) | — |
| 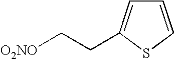 | (CDCl₃): δ 7.20 (dd, 1H), 6.95 (dd, 1H), 6.89 (dd, 1H), 4.64 (t, 2H), 3.23 (t, 2H) | — |
| 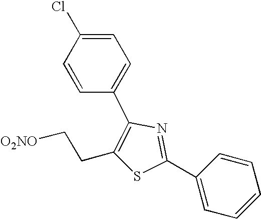 | (CDCl₃): δ 7.96 (m, 2H), 7.59 (m, 2H), 7.45 (m, 5H) 4.68 (t, 2H), 3.37 (t, 2H). | (CDCl₃): δ 166.09, 152.92, 134.52, 133.44, 133.15, 120.49, 130.31, 129.18, 129.01, 126.67, 72.27, 25.37. |
| 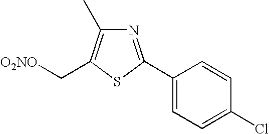 | (CDCl₃): δ 7.85 (d, 2H), 7.41 (d, 2H), 5.60 (t, 2H), 2.53 (s, 3H) | (CDCl₃): δ 167.38, 155.53, 136.76, 131.78, 129.46, 127.94, 121.91, 65.12, 15.5. |
| 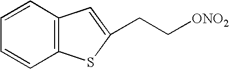 | (CDCl₃): δ 7.78 (m, 1H), 7.76 (m, 1H), 7.31 (m, 2H), 7.11 (s, 1H), 4.71 (t, 2H), 3.29 (t, 2H) | — |
| 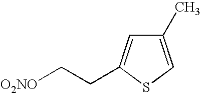 | (CDCl₃): δ 6.70 (s, 1H), 6.56 (s, 1H), 3.78 (t, 2H), 2.09 (t, 2H), 2.19 (s, 3H) | — |
| 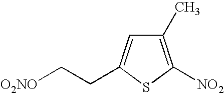 | (CDCl₃): δ 6.74 (s, 1H), 4.67 (t, 2H), 3.12 (t, 2H), 2.59 (s, 3H) | — |

### EXAMPLE 2

Characterization of Cardioprotection in Isolated, Perfused Heart

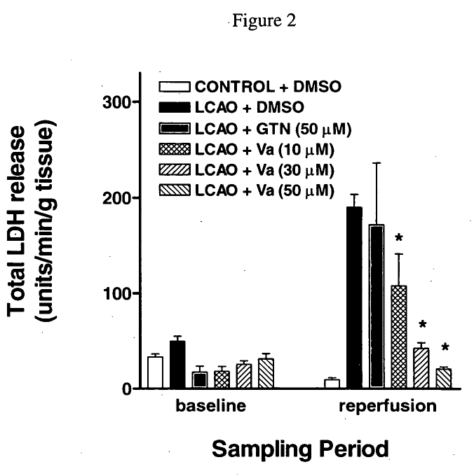

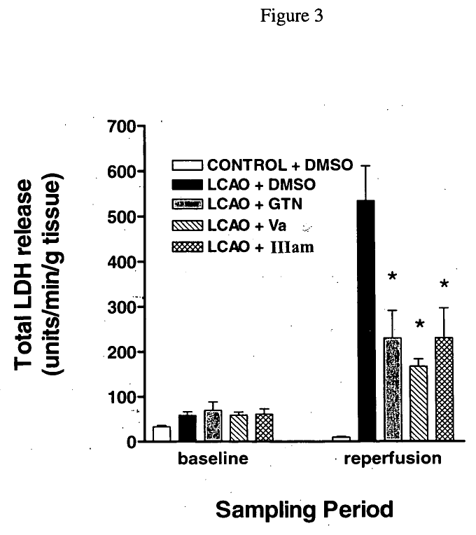

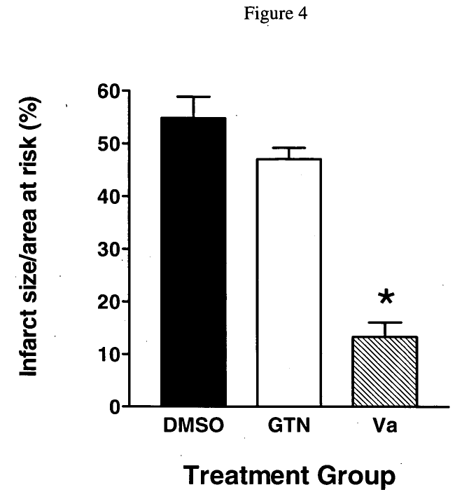

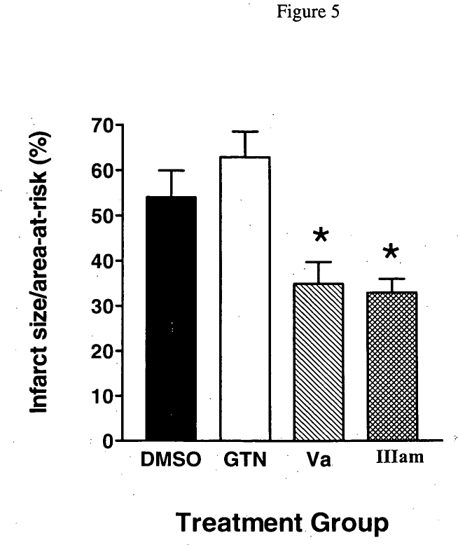

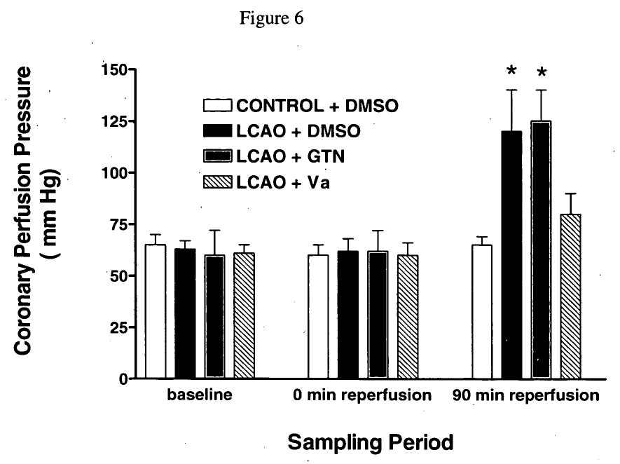

In order to test for potential cardioprotective properties, the effects of Va and compound IIIam were tested in an in vitro model of cardiac ischemia, in which isolated, perfused rat hearts were subjected to transient left coronary artery occlusion (LCAO) followed by reperfusion. Drug treatments [DMSO (drug vehicle), GTN, compound Va or compound IIIam were initiated at two distinct time points: (i) prior to and throughout the 45 minute period of LCAO (protection) or (ii) prior to and throughout the 90 minute reperfusion period (salvage). Drug-induced reduction of lactate dehydrogenase (LDH) release and reduction of infarct size were assessed as measures of cardioprotection. Rat hearts were excised and mounted for retrograde aortic perfusion at a constant flow rate of 6-8 mL/min/g heart weight. The coronary perfusion pressure was monitored by a pressure transducer connected to the perfusion line. To induce regional ischemia, the left coronary artery was occluded for 45 minutes, after which the occlusion was released and the heart reperfused for 90 minutes. At the end of the reperfusion period, the LCA was re-occluded and 0.5 mL of 1% Evan's Blue dye was slowly infused into the heart, via the aotic cannula, to stain the area of myocardium perfused by the patent right coronary artery. Thus the area-at-risk (AAR) for infarction was determined by negative staining. Acute ischemic damage was assessed by measuring the release of the cytosolic enzyme, LDH, into the perfusate, and by quantitation of infarct size by staining for viable tissue using 2,3,5-triphenyltetrazolium chloride (TTC) followed by computerized planimmetry. Infarct size was expressed as infarct area (negative staining after TTC staining) as a percent of the area-at-risk (negative staining after Evan's Blue dye). Left coronary artery occlusion was associated with a 5-10 fold increase in LDH release (FIGS. 2 and 4, solid bars), whereas LDH release was not increased in non-occluded hearts (FIGS. 2 and 3, open bars). When administered prior to and during the period of LCA occlusion (protection protocol), compound Va, in a concentration-dependent manner, significantly reduced LDH release during the subsequent period of reperfusion (FIG. 2, * P<0.01 vs. LCAO+DMSO, one-way ANOVA, Tukey-Kramer post-hoc test), whereas GTN had no effect. Using the same drug infusion protocol, compound Va significantly reduced the size of the myocardial infarct after LCAO and reperfusion (FIG. 4, * P<0.01 vs DMSO-treated hearts, one-way ANOVA, Tukey-Kramer post-hoc test) whereas GTN had no effect. As a measure of the functional recovery of the heart after ischemia/reperfusion injury, the increase in perfusion pressure during the reperfusion period was monitored (FIG. 6). In hearts subjected to LCAO, perfusion pressure was increased by about 2-fold by the end of the reperfusion period. This increase was markedly reduced in hearts treated with compound Va, whereas GTN had no effect. When GTN, compound Va and compound IIIam were infused prior to and throughout the reperfusion period (salvage protocol), all three drugs decreased LDH release during the reperfusion period (FIG. 3, * P<0.01 vs. LCAO+DMSO, one-way ANOVA, Tukey-Kramer post-hoc test). However, only compound Va and compound IIIam reduced infarct size when administered prior to and throughout the reperfusion period (FIG. 5). These results indicate that compound Va reduces the severity of ischemia-reperfusion injury when administered prior to and throughout an acute ischemic insult, and that compounds Va and compound IIIam reduce the severity of ischemia-reperfusion injury when administered just prior to and throughout reperfusion, after a prolonged ischemic insult. However, the prototypical nitrate, GTN, is ineffective at protecting the heart from acute ischemic insult.

### EXAMPLE 3

Neuroprotection Against 6-hydroxydopamine-induced Killing of Dopaminergic Neurons in the Rat Substantia Nigra Pars Compacta

Male Long-Evans rats were anesthetized with sodium pentobarbital, and received stereotaxic, unilateral injections of 6-OHDA (6 μg in 2 μl) into the right substantia nigra pars compacta. Vehicle (dimethylsulfoxide, DMSO) or compound Va were given by subcutaneous injection every hour for 6 hours, beginning 30 minutes before 6-OHDA. Each dose of compound Va was 200 μmol/kg. Two weeks after the administration of 6-OHDA or vehicle, the rats received a single injection of apomorphine (1 mg/kg, s.c.), and contralateral rotations were counted at 15 minute intervals for 60 minutes. In some animals, the brains were fixed, and frozen sections cut for immunocytochemical analysis of tyrosine hydroxylase (TH).

In vehicle-treated animals, apomorphine induced rotations contralateral to the lesion that persisted for the entire 60 minute observation period. In contrast, animals that received compound Va exhibited essentially no rotations over 60 minutes in response to apomorphine injection, suggesting virtually complete protection against the neurotoxic effects of 6-OHDA (Table 3). Immunocytochemical analysis confirmed that compound Va preserved TH-positive neurons in the substantia nigra of 6-OHDA-injected rats (FIG. 7). These data demonstrate that compound Va is a very effective neuroprotective agent against 6-OHDA-induced killing of dopaminergic neurons in the rat substantia nigra pars compacta.

TABLE 3
Apomorphine-Induced Rotations after Unilateral
6-Hydroxydopamine Lesion

| Treatment | Rotations/15 minutes |
|---|---|
| Vehicle | 32 ± 6 (n = 8) |
| Compound Va | 0.2 ± 0.1 (n = 10)* |
| Deprenyl | 0.1 ± 0.1 (n = 4)* |

*p < 0.001 compared to vehicle control, One-way analysis of variance with Bonferroni's Multiple Comparison's test.

Synaptosome Assay The methodology for measurement of thiobarbituric acid reactive substances (TBARS) from synaptosomes was adapted from that of Keller et al. Neuroscience 80: 685-696, 1997. Adult Sprague-Dawley rats (250-300 g) were anesthetized with halothane for 20 sec. and decapitated. The brain was removed and the cerebral cortex separated from white matter. The cerebral cortex was homogenized in a solution containing sucrose 0.32 M, EDTA 2 mM and TRIS.HCl 10 mM, pH 7.2, using a Teflon pestle. The tissue was 5% w/v in the homogenizing buffer. The homogenate was centrifuged for 10 minutes at 310 g at 4° C. The supernatant was then centrifuged for 10 minutes at 20,000 g at 4° C. The pellet was collected, resuspended in Locke's buffer (154 mM NaCl, 5.6 mM KCl, 2.3 mM CaCl₂.2H₂O, 1.0 mM MgCl₂.6H₂O, 3.6 mM NaHCO₃, 5 mM glucose, 5 mM HEPES, pH 7.2) and centrifuged for 10 minutes at 20,000 g at 4° C. The procedure of washing the pellet was repeated 2-3 times in order to reduce transition metal ion concentrations. Finally, the pellet was resuspended in Locke's buffer for use in the lipid peroxidation assay. All assays were performed in triplicate and on three separate synaptosome preparations from different animals.

Potential antioxidants and prooxidants were freshly prepared: in Locke's buffer [FeSO₄; ascorbic acid (AA); Trolox; cysteine]; in 10 mM NaOH [NONOates]; or in organic solvent, such as methanol or DMSO, [α-tocopherol (αTH); nitrates; nitrites; PhSH; lipoic acid (LA); dihydrolipoic acid (LAH₂)]; final dilution ≦2.5% (v/v) organic component). Pro/antioxidants (or solvent vehicle in control experiments) were added to the synaptosome preparation, followed immediately by freshly prepared aqueous FeSO₄ (or buffer in control experiments) and incubated for 30 minutes at 37° C. (air equilibrated). After incubation, TBA reagent (TBA 0.375% w/v, Cl₃CCO₂H 15% w/v, HCl 1M 25% v/v) was added to the homogenate (homogenate: TBA reagent, 1:2 v/v), and the sealed samples were boiled for 15 minutes at approx. 96-100° C. The cooled samples were then centrifuged for 10 minutes at 9,000 g at room temperature. The pink supernatant was transferred into microplates and the absorbance was measured at 530 nm on a Dynex MRX microplate reader. TBA reagent solutions were freshly made and calibrated using solutions of authentic malondialdehyde.

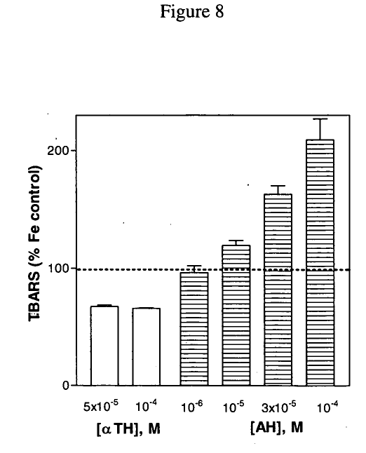

Lipid peroxidation: synaptosome experimental results. Preliminary lipid peroxidation experiments explored the time course of synaptosome lipid peroxidation, incubating homogenate with FeSO₄ (10 μM-150 μM) in Locke's buffer, for time intervals from 15 minutes to 180 minutes (data not shown). Under these experimental conditions, the level of peroxidation, as measured by TBARS, was seen to be below saturation at 30 minutes using 50 μM FeSO₄. The ability of this system to provide concentration dependent lipid peroxidation data was demonstrated using the antioxidant α-tocopherol (αTH), and ascorbic acid (AH), which is known to act as a prooxidant in Fe(II)-induced lipid peroxidation systems (see FIG. 8). These conditions thus were chosen for all further synaptosome experiments.

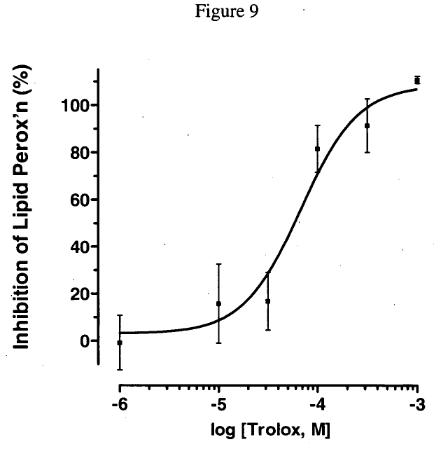

The Fe/synaptosome/TBARS system was designed to provide concentration-response curves for inhibition of lipid peroxidation, which might be quantified by EC₅₀ values. Absolute EC₅₀ values measured in such systems are highly dependent on experimental conditions, and therefore must be benchmarked against well-studied antioxidants, such as Trolox, a water soluble chroman carboxylate derivative of αTH (see FIG. 9).

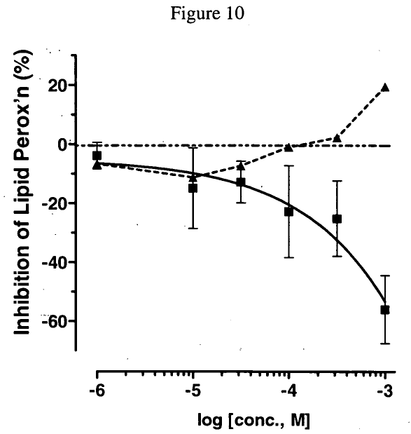

Thiols can display mixed pro- and antioxidant activity towards lipid peroxidation. In particular, in the presence of transition metals, either added to lipid preparations, or adventitious metal ions present in tissue homogenates, thiols may act as prooxidants. The vic-dithiol, dihydrolipoic acid (LAH₂, ▪, solid line in FIG. 10) yielded a concentration dependent prooxidant effect, akin to ascorbic acid, whereas the oxidized disulfide lipoic acid (LA, ▴, dashed line in FIG. 10) showed very modest inhibition of lipid peroxidation at the highest concentration applied. Cysteine (1 mM) was a prooxidant in the presence of FeSO₄ giving 117% of the lipid peroxidation seen in the presence of FeSO₄ alone, whereas PhSH (1 mM) in the presence of FeSO₄, gave 110% of the lipid peroxidation seen in the presence of FeSO₄ alone (data not shown). Of further consideration in analysis of lipid peroxidation data is the requirement for added thiols in experiments with nitrates. Clearly, any antioxidant effect of nitrates may be masked by the prooxidant effect of the adjuvant thiol. Thus, it was chosen to present the data as “percentage inhibition of lipid peroxidation”, by normalizing TBARS measurements to: (i) 100% inhibition of lipid peroxidation (corresponding to TBARS in the presence of vehicle and absence of FeSO₄); and, (ii) 0% inhibition of lipid peroxidation (corresponding to TBARS in the presence of vehicle, FeSO₄, and any added thiol). This methodology and protocol was applied uniformly to all experiments graphed in FIGS. 9-17.

The antioxidant trolox yielded a potent concentration dependent reduction in TBARS products with EC₅₀=6.8×10⁻⁵ M (FIG. 9), which can be contrasted to the effect of lipoic acid (LA) or dihydrolipoic acid (LAH₂, see FIG. 10).

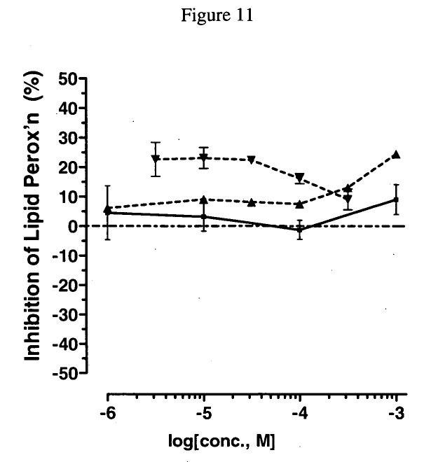

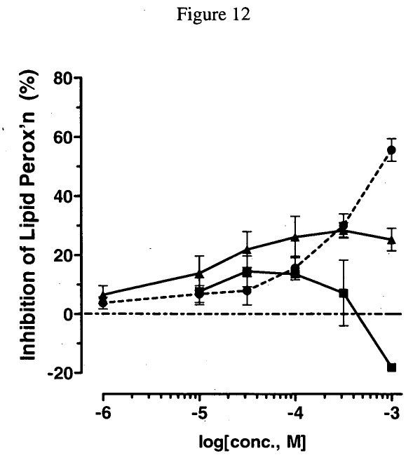

GTN alone had no effect on Fe-induced lipid peroxidation (data not shown), nor did varied concentrations of GTN have any significant effect in the presence of added LAH₂ (1 mM) (FIG. 11). Indeed, GTN produced no significant effect on lipid peroxidation with any thiol used (e.g. cysteine, PhSH), over the effect of the thiol itself (see GTN with LAH₂, ▪, solid line in FIG. 11). However, compound Va (▴, dashed line, FIG. 11) did inhibit lipid peroxidation at higher concentrations in the presence of LAH₂. Further, in contrast to GTN, compound Va inhibited TBARS formation with the water soluble thiol, cysteine, and the more lipophilic thiophenol (PhSH) (see Va+cysteine, ▪, solid line or Va+PhSH, ▴, solid line in FIG. 12).

Addition of PhSH (1 mM) yielded a concentration dependent inhibition curve for lipid peroxidation: EC₅₀=1.4×10⁻⁵ M. At high millimolar concentrations, compound Va showed some prooxidant activity in the presence of cysteine.

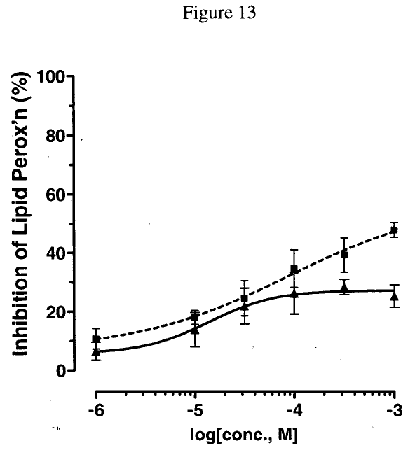

Data for inhibition of iron-induced lipid peroxidation by compound IVr can be fit to a curve leading to 100% efficacy with an EC₅₀ of 1.2×10⁴M (or fitted without constraint on efficacy to EC₅₀=2.7×10⁻⁴ M; efficacy=78%). Moreover, compound Va, in the absence of thiol, showed a modest inhibition of lipid peroxidation, more pronounced at lower concentrations (see FIG. 13). This antioxidant effect is clearly not an ubiquitous property of disulfides, since lipoic acid (LA) does not show such properties in the identical assay (FIG. 10).

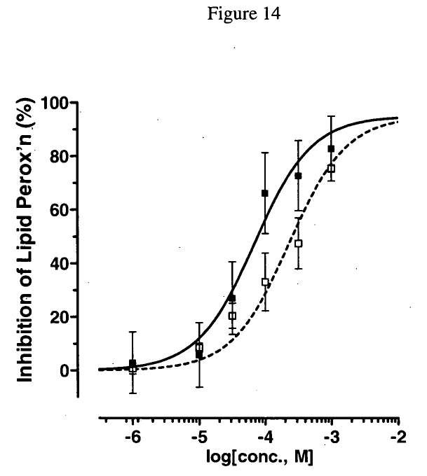

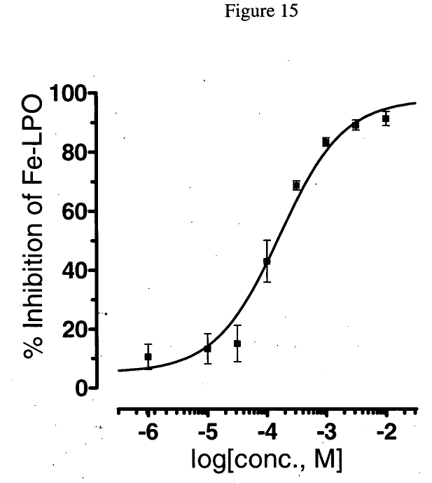

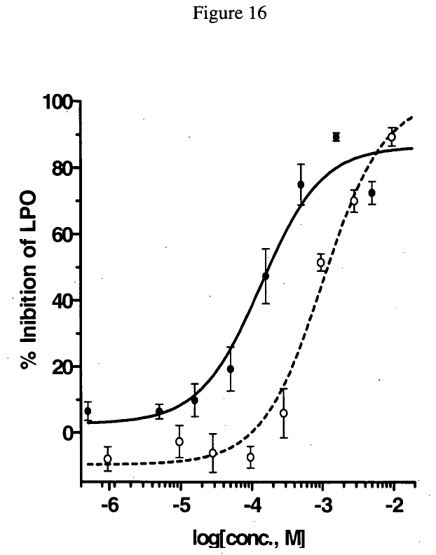

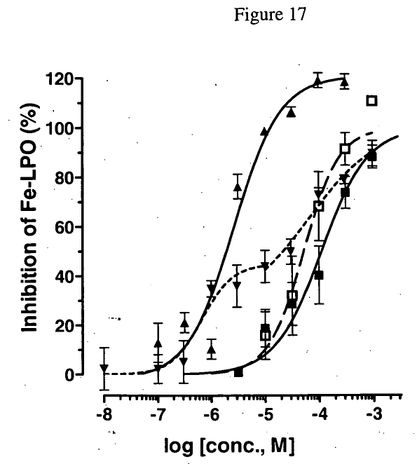

Concentration response curves were derived from TBARS data for the NO donor NONOate, Sper/NO (spermine NONOate, dashed line), and for DEA/NO (diethylamine NONOate, solid line) (see FIG. 14). The potency and efficacy of inhibition of lipid peroxidation by Sper/NO and by IAN (see FIG. 15) were both observed to be approximately identical (EC₅₀ IAN=1.6×10⁻⁴ M, Sper/NO=2×10⁻⁴ M). TBARS measured for the compound IVs (dashed lines in FIG. 16), in the absence of any adjuvants, revealed similar efficacy for this compound compared to Sper/NO and IAN, but a potency lower by an order of magnitud (EC₅₀ (synaptosomes)=1.0×10⁻³ M, (liposomes)=1.3×10⁻⁴ M). The observed behavior of compounds IVs and IVr was similar, but measurements on compound IVr could not be extended to higher concentrations because of solubility. The potency and efficacy of inhibition of lipid peroxidation by compounds Vbb (▾, solid line in FIG. 17) and Vbc (▪, solid line in FIG. 17) were also measured and found to be 2.0×M⁻⁵ M for Vbb and and 7×10⁻⁷ M and 7×10⁻⁵ M for Vbc.

### REFERENCES

Abe, K., C. Takeyama, K. Yoshimura, “Effects of S-8510, a novel benzodiazepine receptor partial inverse agonist, on basal forebrain lesioning-induced dysfunction in rats”, Eur. J. Pharmacol. 347 (1998) 145-152.
Arancio, O., E. R. Kandel, R. D. Hawkins, “Activity-dependent long-term enhancement of transmitter release by presynaptic 3′,5′-cyclic GMP in cultured hippocampal neurons”, Nature 376 (1995) 74-80.
Barger, S. W., R. R. Fiscus, P. Ruth, F. Hofmannii, M. P. Mattson, “Role of cyclic GMP in the regulation of neuronal calcium and survival by secreted forms of β-amyloid precursor protein”, J. Neurochem. 64 (1995) 2087-2096.
Berge et al., “Pharmaceutical Salts”, J. Pharm. Sci. 66 (1977) 1-19.
Bernabeu, R., N. Schroder, J. Quevedo, M. Cammarota, I. Izquierdo, J. H. Medina, “Further evidence for the involvement of a hippocampal cGMP/cGMP-dependent protein kinase cascade in memory consolidation”, NeuroReport 8 (1997) 2221-2224.
Bernabeu, R., P. Schmitz, M. P Faillace, I. Izquierdo, J. H. Medina, “Hippocampal cGMP and cAMP are differentially involved in memory processing of inhibitory avoidance learning”, NeuroReport 7 (1996) 585-588.
Bloeman, P. G. et al., FEBS Lett., 357 (1995) 140.
Briscoe et al., Am. J. Physiol. 1233 (1995) 134.
Bullock, R., A Zauner, J. J. Woodward, J. Myseros, S. C. Choi, J. D. Ward, A. Marmarou, H. F. Young, “Factors affecting excitatory amino acid release following severe human head injury”, J. Neurosurg. 89 (1998) 507-518.
Chan, P. H., M. Kawase, K. Murakami, S. F. Chen, Y. Li, B. Calagui, L. Reola, E. Carlson, C. J. Epstein, “Overexpression of SOD1 in transgenic rats protects vulnerable neurons against ischemic damage after global cerebral ischemia and reperfusion”, J. Neurosci. 18 (1998) 8292-8299.
Chen, J., T. Nagayama, K. Jin, R. A. Stetler, R. L. Zhu, S. H. Graham, R. P. Simon, “Induction of caspase-3-like protease may mediate delayed neuronal death in the hippocampus after transient cerebral ischemia”, J. Neurosci. 18 (1998) 4914-4928.
Cohen, G. M., “Caspases: the executioners of aopotosis”, Biochem. J. 326 (1997) 1-16.
Du, Y., K. R. Bales, R. C. Dodel, E. Hamilton-Byrd, J. W. Horn, D. L. Czilli, L. K. Simmons, B. Ni, S. M. Paul, “Activation of a caspase-3-related cysteine protease is required for glutamate-mediated apoptosis of cultured cerebellar granule neurons”, Proc. Natl. Acad. Sci. USA 94 (1997) 11657-11662.
Endres, M., S. Namura, M. Shimizu-Sasamata, C. Waeber, L. Zhang, T. Gomez-Isla, B. T. Hyman, M. A. Moskowitz, “Attenuation of delayed neuronal death after mild focal ischemia in mice by inhibition of the caspase family”, J. Cereb. Blood Flow Metab. 18 (1998) 238-247.
Estevez, A. G., N. Spear, J. A. Thompson, T. L. Cornwell, R. Radi, L. Barbeito, J. S. Beckman, “Nitric oxide-dependent production of cGMP supports the survival of rat embryonic motor neurons cultured with brain-derived neurotrophic factor”, J. Neurosci. 18 (1998) 3708-3714.
Farinelli, S. E., D. S. Park, L. A. Greene, “Nitric oxide delays the death of trophic factor-deprived PC12 cells and sympathetic neurons by a cGMP-mediated mechanism”, J. Neurosci. 16 (1996) 23-25-2334.
Furukawa, K., S. W. Barger, E. M. Blalock, M. P. Mattson, “Activation of K⁺ channels and suppression of neuronal activity by secreted β-amyloid precursor protein”, Nature 379 (1996) 74-78.
Gaetani, P., A. Pasqualin, R. Rodriguez y Baena, E. Borasio, F. Marzatico, “Oxidative stress in the human brain after subarachnoid hemorrhage”, J. Neurosurg. 89 (1998) 748-754.
Goda, H., H. Ooboshi, H. Nakane, S. Ibayashi, S. Sadoshima, M. Fujishima, “Modulation of ischemia-evoked release of excitatory and inhibitory amino acids by adenosine A1 receptor agonist”, Eur. J. Pharmacol. 357 (1998) 149-155.
Gottron, F. J., H. S. Ying, D. W. Choi, “Caspase inhibition selectively reduces the apoptotic component of oxygen-glucose deprivation-induced cortical neuronal cell death”, Mol. Cell. Neurosci. 9 (1997) 159-169.
Haviv, R., L. Lindenboim, H. Li, J. Yuan, R. Stein, “Need for caspases in apoptosis of trophic factor-deprived PC12 cells”, J. Neurosci. Res. 50 (1997) 69-80.
Higashi et al., Neuropathol. Appl. Neurobiol. 21 (1995) 480-483.
Huang, F. P., L. F. Zhou, G. Y. Yang, “Effects of mild hypothermia on the release of regional glutamate and glycine during extended transient focal cerebral ischemia in rats”, Neurochem. Res. 23 (1998) 991-996.
Ibarrola, D., H. Seegers, A. Jaillard, M. Hommel, M. Decorps, R. Massarelli, “The effect of eliprodil on the evolution of a focal cerebral ischaemia in vivo”, Eur. J. Pharmacol. 352 (1998) 29-35.
Jiang et al., J. Cereb. Blood Flow Metab. 18 (1998) 758-767.
Kesner, NIDA Res. Monographs 97 (1990) 22-36.
Kim, Y. M., R. V. Talanian, T. R. Billiar, “Nitric oxide inhibits apoptosis by preventing increases in caspase-3-like activity via two distinct mechanisms”, J. Biol. Chem. 272 (1997) 31138-31148.
Louw, R., H. P. W. Vermeeren, J. J. A. Van Asten, W. J. Ultee, J. Chem. Soc., Chem. Comm. (1976) 496-497
Macdonald, R. L., M. Stoodley, “Pathophysiology of cerebral ischemia”, Neurol. Med. Chir. (Tokyo) 38 (1998) 1-11.
Mattson and Scheff, J. Neurotrauma 11 (1994) 3-33.
Mizuno, A., K. Umemura, M. Nakashima, “Inhibitory effect of MCI-186, a free radical scavenger, on cerebral ischemia following rat middle cerebral artery occlusion”, Gen. Pharmacol. 30 (1998) 575-578.
Mordenti, “Man versus beast: Pharmacokinetic scaling in mammals”, J. Pharm. Sci. 75 (1986) 1028-1040.
Morgan et al., “Approaches to the discovery of non-peptide ligands for peptide receptors and peptidases”, In Ann. Rep. Med. Chem. (Virick F. J., et al.) (1989) pp. 243-253, Academic Press, San Diego, Calif.
Namura, S., J. Zhu, K. Fink, M. Endres, A Srinivasan, K. J. Tomaselli, J. Yuan, M. A. Moskowitz, “Activation and cleavage of caspase-3 in apoptosis induced by experimental cerebral ischemia”, J. Neurosci. 18 (1998) 3659-3668.
Ni, B., X. Wu, Y. Su, D. Stephenson, E. B. Smalstig, J. Clemens, S. M. Paul, “Transient global forebrain ischemia induces a prolonged expression of the caspase-3 mRNA in rat hippocampal CA1 pyramidal neurons”, J. Cereb. Blood Flow Metab. 18 (1998) 248-256.
Nicholson, D. W., N. A. Thornberry, “Caspases: killer proteases”, Trends Biochem. Sci. 22 (1997) 299-306.
O'Neill, M. J., A. Bond, P. L. Ornstein, M. A. Ward, C. A. Hicks, K. Hoo, D. Bleakman, D. Lodge, “Decahydroisoquinolines: novel competitive AMPA/kainate antagonists with neuroprotective effects in global cerebral ischaemia”, Neuropharmacol. 37 (1998) ¹²¹I-1222.
Ouellette, R. J., R. J. Bertsch, J. Org. Chem. 41 (1976) 2782-2783.
Owais, M. et al., Antimicrob. Agents Chemother., 39 (1995) 180.
Pallares, M., M. Darnaudery, J. Day, M. Le Moal, W. Mayo, “The neurosteroid pregnenolone sulfate infused into the nucleus basalis increases both acetylcholine release in the frontal cortex or amygdala and spatial memory”, Neurosci. 87 (1998) 551-558.
Ranade, V. V., J. Clin. Pharmacol. 29 (1989) 685.
Strejan et al., J. Neuroimmunol. 7 (1984) 27.
Tagami, M., K. Yamagata, K. Ikeda, Y. Nara, H. Fujino, A. Kubota, F. Numano, Y. Yamori, “Vitamin E prevents apoptosis in cortical neurons during hypoxia and oxygen reperfusion”, Lab. Invest. 78 (1998) 1415-1429.
Umemura, K., A. Shimakura, M. Nakashima, “Neuroprotective effect of a novel AMPA receptor antagonist, YM90K, in a rat focal cerebral ischaemia”, Brain Res. 773 (1997) 61-65.
Umezawa et al., Biochem. Biophys. Res. Commun. 153 (1988) 1038.
Venault, P. G., Chapouthier, L., Prado de Carvalho and Rossier, J., Encephale, 18 (1992) 655.
Wittkowsky, Pharmacotherapy 18 (1998) 945-1005.
Wu, J., Y. Wang, M. J. Rowan, R. Anwyl, “Evidence for involvement of the cGMP-protein kinase G signaling system in the induction of long-term depression, but not long-term potentiation, in the dentate gyrus in vitro”, J. Neurosci. 18 (1998) 3589-3596.
Yang, K., J. D. Artz, J. Lock, C. Sanchez, B. M. Bennett, A. B. Fraser, G. R. Thatcher, J. Chem. Soc., Perkin Trans. 1 (1996) 1073-1075.
Yang, Y. L., W. H. Pan, T. H. Chiu, M. T. Lin, “Striatal glutamate release is important for development of ischemic damage to strital neurons during rat heatstroke”, Brain Res. 795 (1998) 121-127.

All publications and patents cited in this specification are hereby incorporated by reference herein as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A nitrate ester having the general formula: 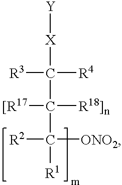
or a pharmaceutically acceptable salt thereof, wherein:
each of m and n is, independently, an integer from 0 to 10;
each of R³, R⁴, R¹⁷ is, independently, hydrogen, a nitrate group, or A;
R¹ is hydrogen or A;
where A is selected from: a substituted or unsubstituted aliphatic group having from 1 to 24 carbon atoms in the chain, which optionally contains 1 to 4 O, S, NR⁶, and/or unsaturations in the chain, optionally bearing from 1 to 4 hydroxy, nitrate, amino, aryl, or heterocyclic groups; an unsubstituted or substituted cyclic aliphatic moiety having from 3 to 7 carbon atoms in the aliphatic ring, which optionally contains 1 to 2 O, S, NR⁶, and/or unsaturations in the ring, optionally bearing from 1 to 4 hydroxy, nitrate, amino, aryl, or heterocyclic groups; an unsubstituted or substituted aliphatic moiety comprising a linkage from 0 to 5 carbon atoms between R¹ and R³ and/or between R¹⁷ and R⁴, which optionally contains 1 to 2 O, S, NR⁶, and/or unsaturations in the linkage, optionally bearing from 1 to 4 hydroxy, nitrate, amino, aryl, or heterocyclic groups; a substituted or unsubstituted aliphatic group having from 1 to 24 carbon atoms in the chain, containing linkages selected from C═O, C═S, and C═NOH, which optionally contains 1 to 4 O, S, NR⁶, and/or unsaturations in the chain, optionally bearing from 1 to 4 hydroxy, nitrate, amino, aryl, or heterocyclic groups; a substituted or unsubstituted aryl group; a substituted or unsubstituted heterocyclic group; an amino group selected from alkylamino, dialkylamino, cyclic amino, cyclic diamino, cyclic triamino, arylamino, diarylamino, and alkyarylamino; a hydroxy group; an alkoxy group; and a substituted or unsubstituted aryloxy group;
each of R², R⁵, R¹⁸ is, independently, hydrogen, A, or X—Y;
where X is F, Br, Cl, NO₂ CH₂, CF₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂H, PO₂M, P(O)(OR¹⁴), P(O)(R¹³), SO, SO₂, C(O)(SR¹³), SR⁵, SSR⁷, or SSR⁵;
Y is F, Br, Cl, CH₃, CF₂H, CF₃, OH, NH₂, NHR⁶, NR⁶R⁷, CN, NHOH, N₂H₃, N₂H₂R¹³, N₂HR¹³R¹⁴, N₃, S, SCN, SC(═NH)N(R¹⁵)₂, SC(═NH)NHR¹⁵SC(O)N(R¹⁵)₂, SC(O)NHR¹⁵ SO₃M, SH, SR⁷, SO₂M, S(O)R⁸, S(O)₂R⁹, S(O)OR⁸, S(O)₂OR⁹, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O)R¹², C(O)(OR¹³) C(O)(SR¹³), SR⁵, SSR⁷, or SSR⁵, or does not exist;
each of R⁶, R⁷, R⁸, R⁹, R¹¹, R¹², R¹³, R¹⁴, R¹⁵, R¹⁶ is, independently, an alkyl or acyl group containing 1-24 carbon atoms, which may contain 1-4 ONO₂ substituents; a C₁-C₆ connection to R¹-R⁴ in a cyclic derivative; a hydrogen, a nitrate group, or A; and
M is H, Na⁺, K⁺, NH₄+, or N+HₖR¹¹₍₄₋ₖ₎ where k is 0 to 3, or other pharmaceutically acceptable counterion;
with the proviso that:
when m=0; n=1;
each of R¹⁸ and R³ is, independently, H, a nitrate group, or a C₁-C₄ alkyl chain, which may include one 0 linking R¹⁸ and R³ to form a pentosyl, hexosyl, cyclopentyl, or cyclohexyl ring, which ring optionally bears a hydroxyl substituent;
each of R¹⁷ and R⁴ is, independently H, a nitrate group, a C₁-C₄ alkyl, optionally bearing 1 to 3 nitrate groups, or an acyl group (—C(O)R⁵);
each of R⁵, R⁶, R⁸, R⁹, R¹², R¹³, R¹⁴, R¹⁵, R¹⁶is, independently, an alkyl group containing 1 to 12 carbon atoms, which may contain 1 to 4 ONO₂ substituents; or a C₁ or C₂ connection to R¹⁸, R¹⁷, or R³ in a cyclic derivative;
each of R⁷ and R¹¹ is, independently, a C₁ to C₈ alkyl or acyl group;
M is H, Na⁺, K⁺, NH₄⁺, or N⁺HₖR¹¹₍₄₋ₖ₎ where k is 0 to 3; and
X is CH₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂M, P(O)(OR¹⁴), P(O)(R¹³), SO, SO₂, C(O)(SR¹³), or SSR⁴;
then Y is not CN, N₂H₂R¹³, N₂HR¹³R¹⁴, N₃, SCN, SC(═NH)N(R¹⁵)₂, SC(═NH)NHR¹⁵, SC(O)N(R¹⁵)₂, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R¹¹, C(O)R¹², C(O)(SR¹³), SR⁴, SR⁵, or SSR⁵, or Y does not exist.

2. The compound of claim 1 selected from the following: 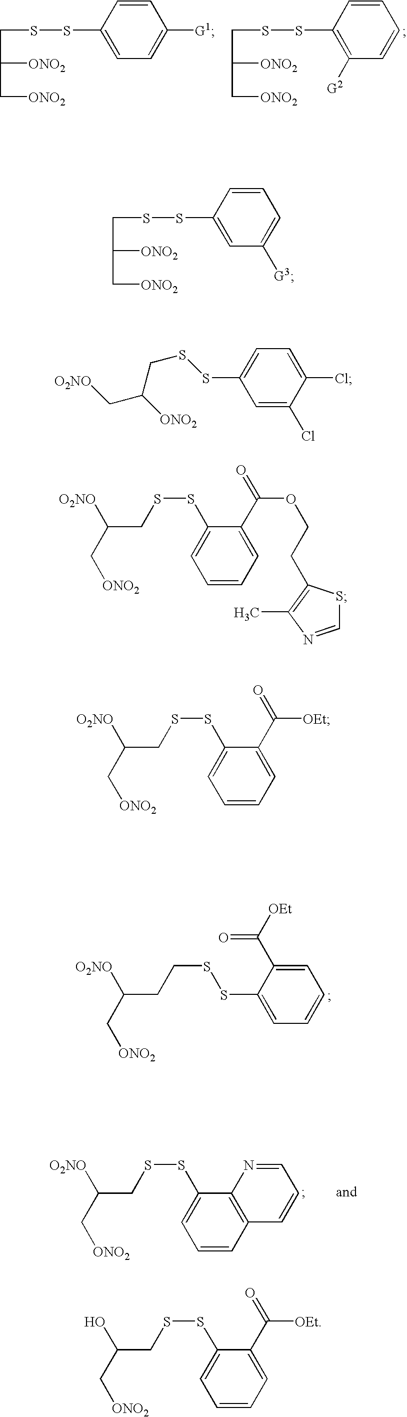
wherein G¹ is Me, OMe, Cl, NO₂, Br, or H; G² is CO₂Et, CO₂H, CO₂Me, CONH₂, or CO(CH₂)₂NEt₂, and G³ is Cl, OMe, or CONH₂.

3. The compound of claim 1 selected from the following:

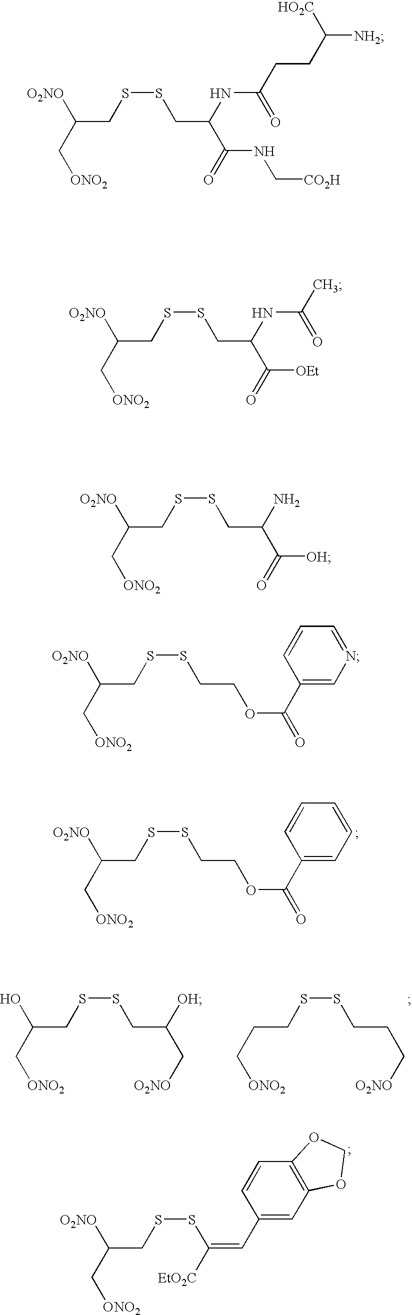

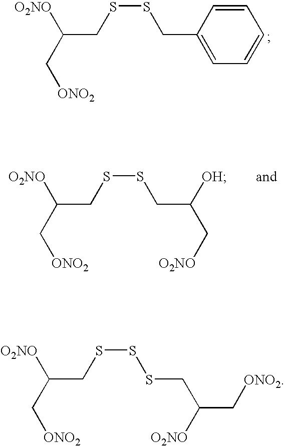

4. The compound of claim 1 selected from the following: 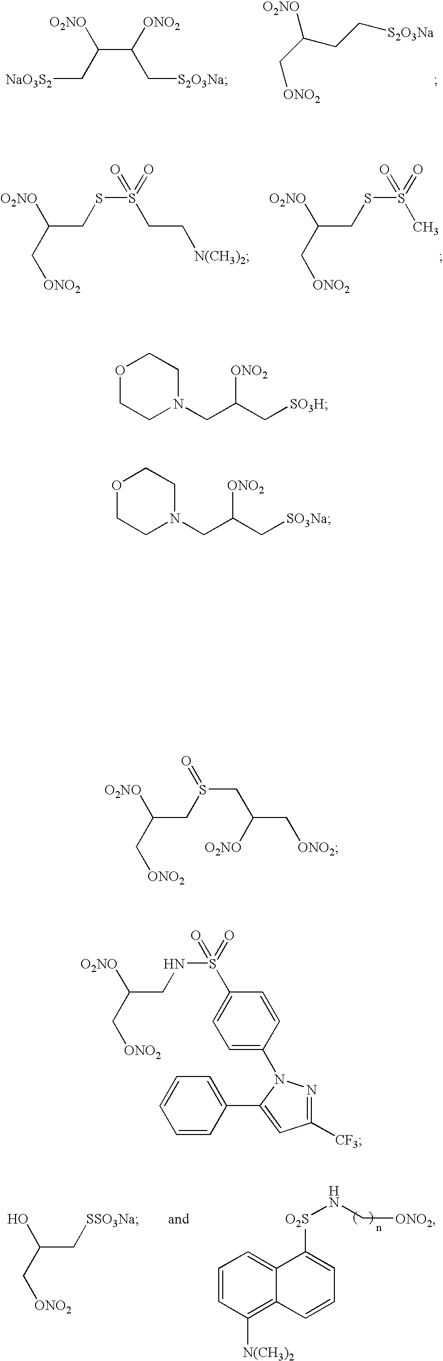
wherein n is 2 or 3.

5. The compound of claim 1 selected from the following:

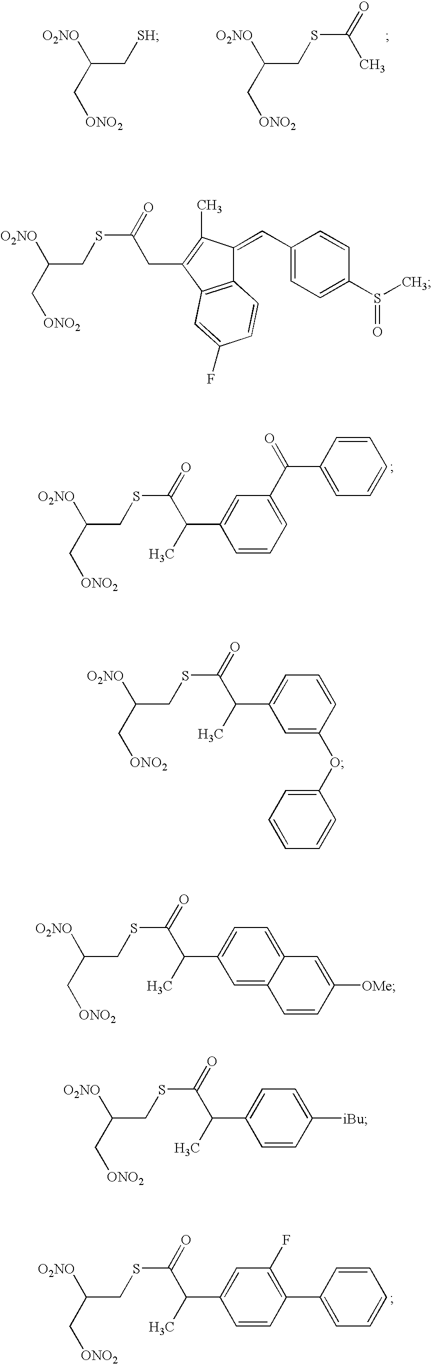

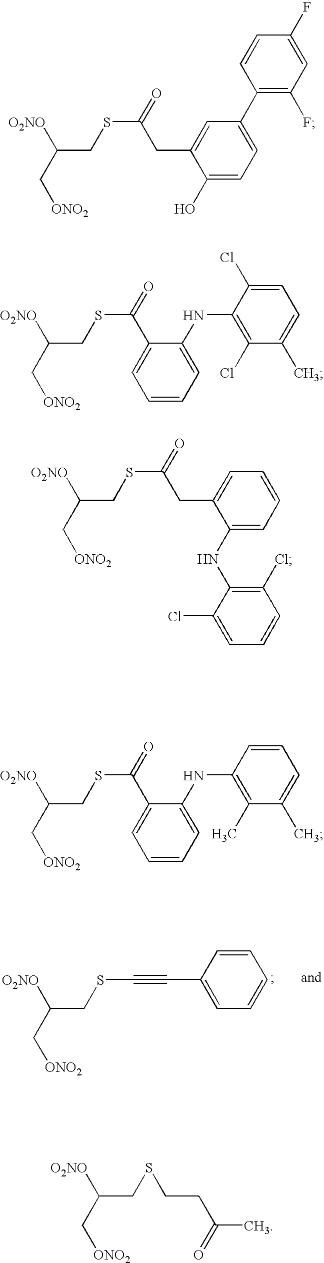

6. The compound of claim 1 selected from the following:

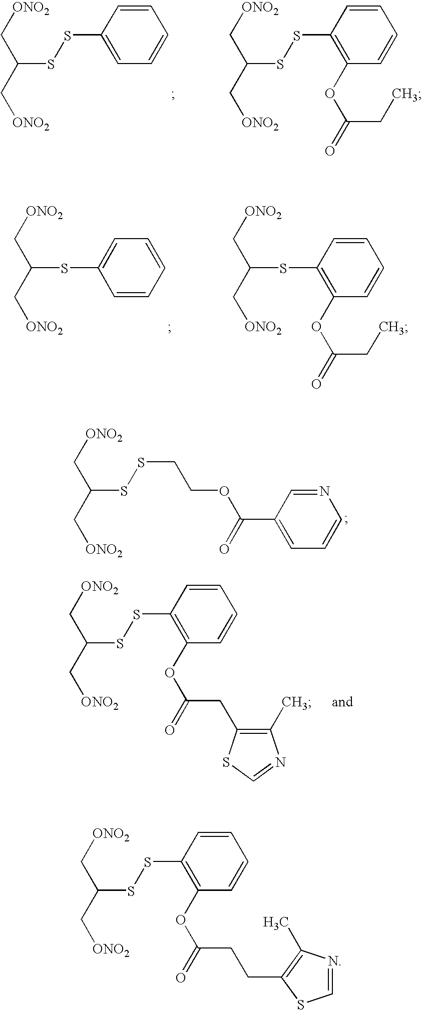

7. The compound of claim 1 selected from the following:

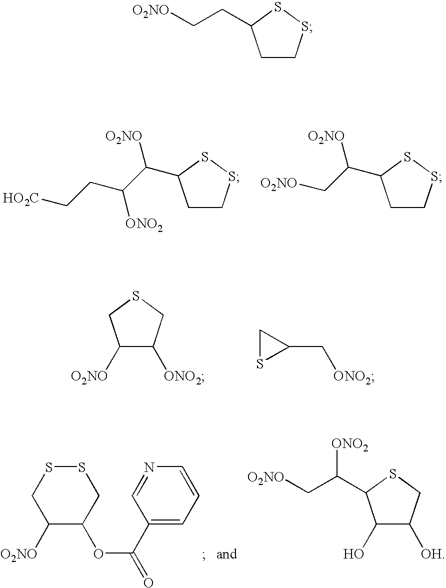

8. A nitrate ester having the general formula: 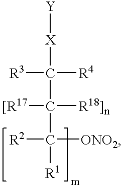
or a pharmaceutically acceptable salt thereof; containing from 1 to 3 nitrate groups and an S atom in proximity to a nitrate group,
wherein
each of m and n is, independently, an integer from 0 to 10;
R¹ is a hydrogen or A;
each of R², R⁵, and R¹⁸ is, independently, hydrogen or A;
each of R³, R⁴, and R¹⁷, is independently, a hydrogen, a nitrate group, or A;
each of R⁶, R⁷, R⁸, R⁹, R¹¹, R¹², R¹³, R¹⁴, R¹⁵ and R¹⁶ is, independently, A, a hydrogen, a nitrate group, or a C₁-C₂₄ alkyl or acyl group, optionally containing 1-4 ONO₂ substituents or a C₁-C₆ linkage to R¹, R², R³, or R⁴ in cyclic derivatives;
each of R⁷ and R¹¹ is, independently, a substituted or unsubstituted C₁-C₈ alkyl or acyl group;
A is selected from:
a C₁-C₂₄ alkyl group, which optionally contains 1 to 4 O, S, NR⁶, and/or unsaturations in the chain, optionally bearing from 1 to 4 hydroxy, Cl, F, amino, unsubstituted or substituted aryl, unsubstituted or substituted heteroaryl, or unsubstituted or substituted heterocyclic groups, or 1-2 nitrate groups;
a C₃-C₂₄ alkyl group, containing 1-5 C═O, C═S, or C═NOR⁷ linkages, which optionally contains 1 to 4 O, S, NR⁶, and/or unsaturations in the carbon chain, optionally bearing from 1 to 4 hydroxy, nitrate, Cl, F, amino, unsubstituted or substituted aryl, unsubstituted or substituted heteroaryl, or unsubstituted or substituted heterocyclic groups;
a C₃-C₇ linkage to any of R¹, R², R³, R⁴, or R¹⁷, forming an aliphatic ring, which optionally contains 1 to 2 O, S, NR⁶, and/or unsaturations in the linkage, optionally bearing from 1 to 6 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
a C₀-C₅ linkage to or between any of R¹, R³, R⁴, or R¹⁷, which optionally contains 1 to 2 O, S, NR⁶, and/or unsaturations in the linkage, bearing two or more substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
an unsubstituted C₀-C₅ linkage to or between any of R¹, R³, and R⁴, which optionally contains 1 to 2 non-adjacent O, S, NR⁶, and/or unsaturations in the linkage;
a C₁-C₅ linkage to or between any of R¹, R³, R⁴, and R¹⁷ containing 1 to 2 C═O, C═S, or C═NOR⁷ linkages, which optionally contains 1 to 2 O, S, NR⁶, and/or unsaturations in the linkage, optionally bearing from 1 to 4 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
a substituted or unsubstituted aryl group;
a substituted or unsubstituted heteroaryl group;
a substituted or unsubstituted heterocyclic group;
an amino, cyclic amino, diamino, triamino, alkylamino, dialkylamino, arylamino, diarylamino, or alkylarylamino group;
a hydroxy group;
an alkoxy group; and
a substituted or unsubstituted aryloxy group;
X is F, Br, NO₂, CH₂ CF₂, O, NH, NMe, NHOH, N₂H₃, N₂H₂R¹³, N₂HR¹³R¹⁴ , N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)R⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷, or does not exist;
Y is F, Br, CH₃, CF₂H, CF₃, OH, NH₂, NHR⁶, NR⁶R⁷, NHOH, N₂H₃, N₂H₂R¹³, N₂HR¹³R¹⁴, N₃, SC(═NH)N(R¹⁵)₂, SC(═NH)NHR¹⁵, SC(O)N(R¹⁵)₂, SC(O)NHR¹⁵, SH, SR⁷, SO₂M, S(O)R⁸, S(O)R⁵, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R⁵, C(O)(OR¹³), C(O)(SR¹³), C(S)R⁵, C(S)R¹², C(S)OR¹², or does not exist; and
M is H, Na⁺, K⁺, NH₄⁺, N⁺HₖR¹¹₍₄₋ₖ₎ where k is 0-3, or other pharmaceutically acceptable counterion;
and with the proviso that,
when m=0 and n=1;
each of R¹⁸ and R³ is, independently, a hydrogen, a nitrate group, or a C₁-C₄ alkyl chain, which may include one O, linking R¹⁸ and R³ together to form a pentosyl, a hexosyl, a cyclopentyl, or a cyclohexyl ring, said ring optionally bearing from 1-4 hydroxyl substituents;
each of R¹⁷ and R⁴ is, independently, a hydrogen, a nitrate group, a C₁-C₄ alkyl group, optionally bearing from 1-3 nitrate groups, or an acyl group (—C(O)R⁵);
each of R⁵, R⁶, R⁸, R⁹, R¹², R¹³, R¹⁴ , R¹⁵, and R¹⁶ is, independently, a C₁-C₁₂ alkyl group, optionally bearing from 1-4 ONO₂ substituents, or a C₁-C₂ alkyl linkage to R¹⁸, R¹⁷, or R³;
X is F, Br, NO₂, CH₂ CF₂, O, NH, NMe, NHOH, N₂H₃, N₂H₂R¹³, N₂HR¹³R¹⁴ , N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷; and
Y is not CN, N₂H₂R¹³, N₂HR¹³R¹⁴, N₃, SCN, SC(═NH)N(R¹⁵)₂, SC(═NH)NHR¹⁵, SC(O)N(R¹⁵)₂, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R⁵, C(O)R¹², C(O)(SR¹³), SR⁴, SR⁵, or SSR⁵, or Y does not exist.

9. The compound of claim 8 selected from the following:

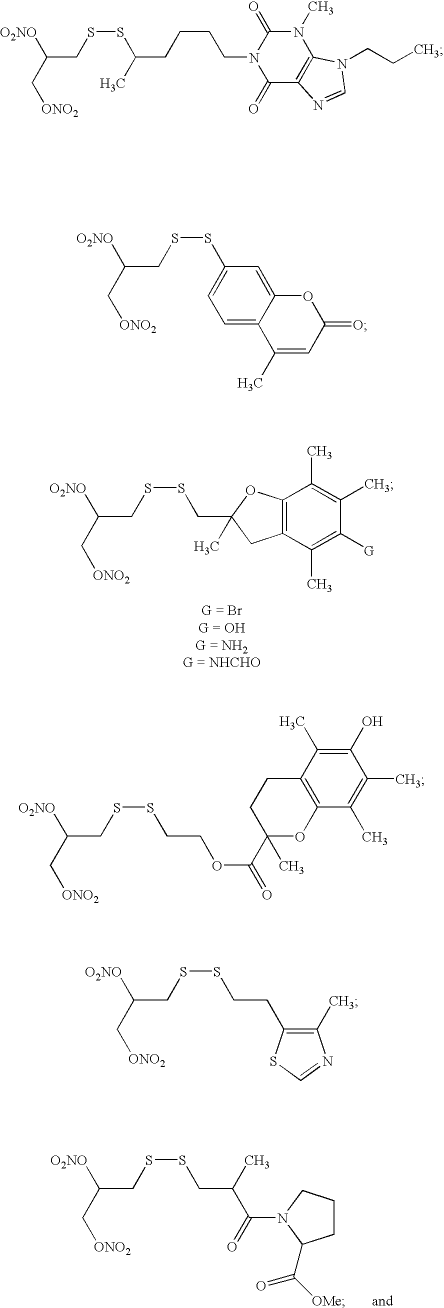

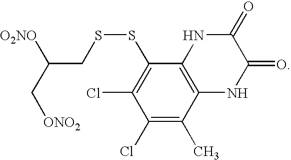

10. The compound of claim 8 selected from the following:

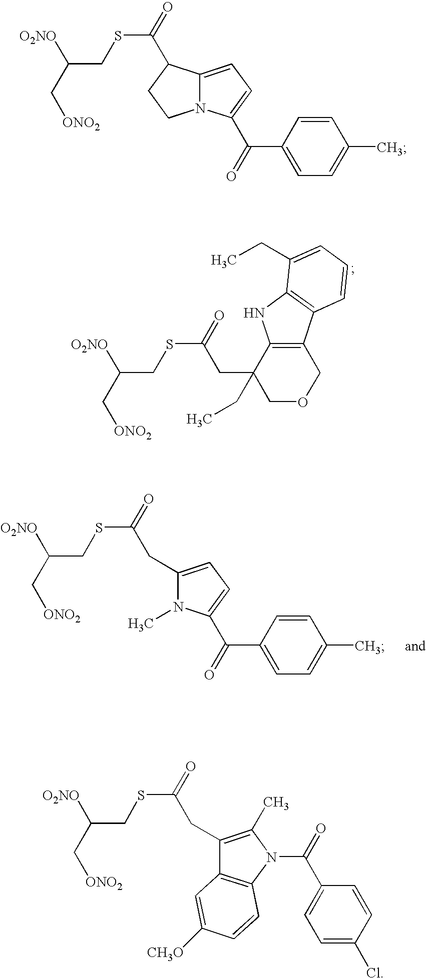

11. The compound of claim 8 selected from the following:

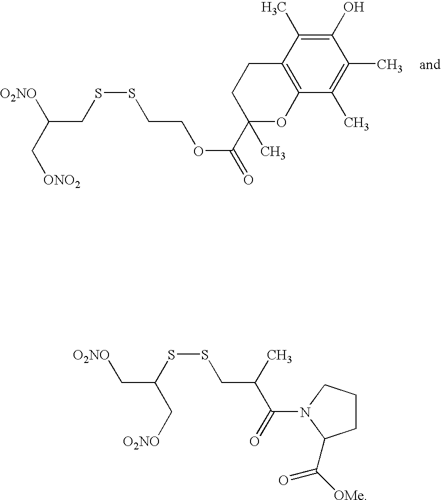

12. The compound of claim 8 selected from the following:

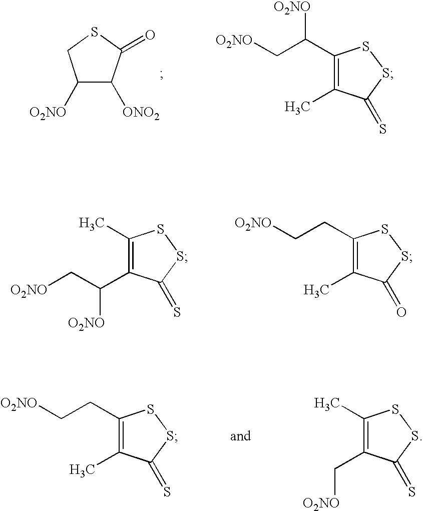

13. The compound of claim 8 selected from the following:

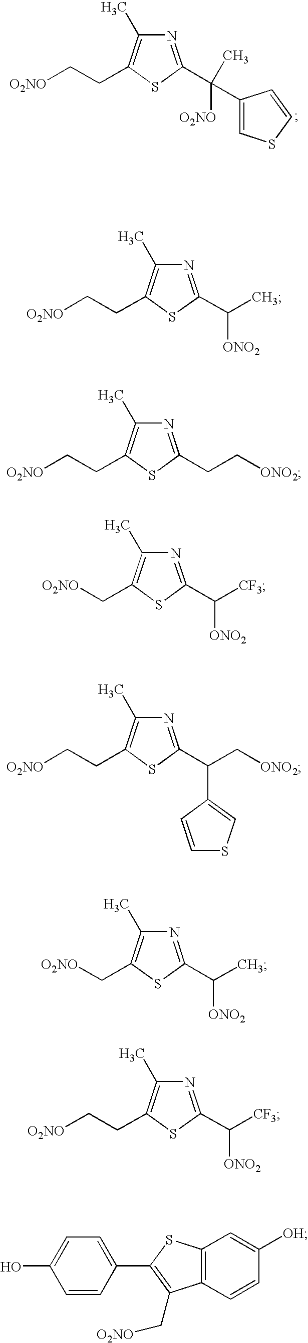

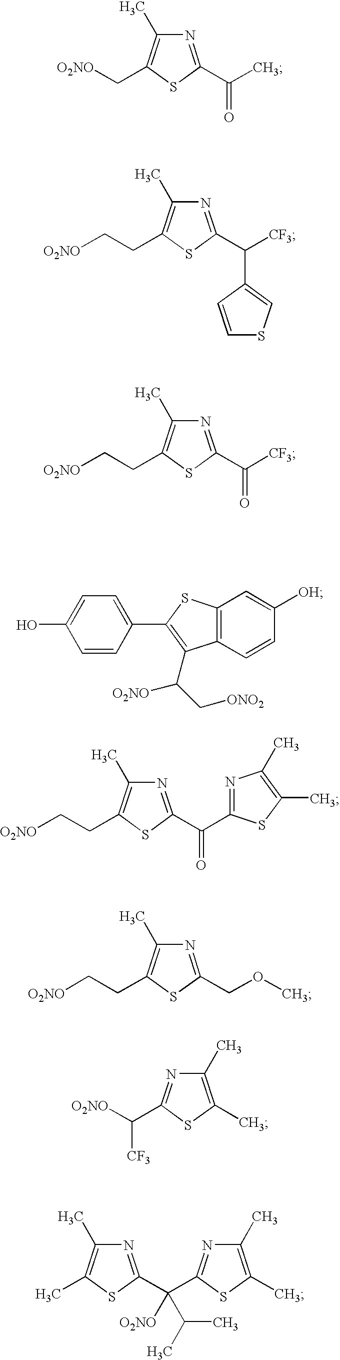

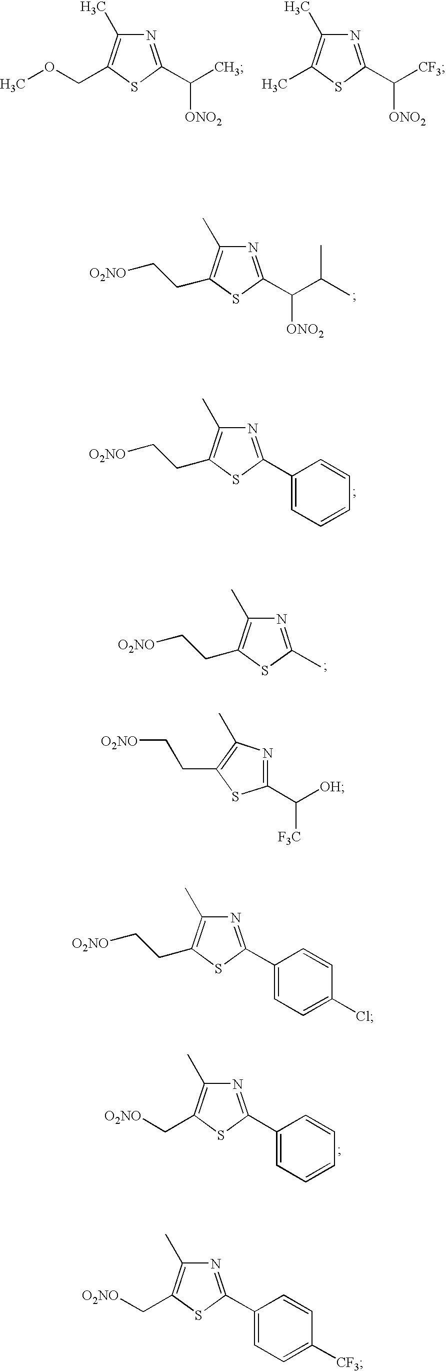

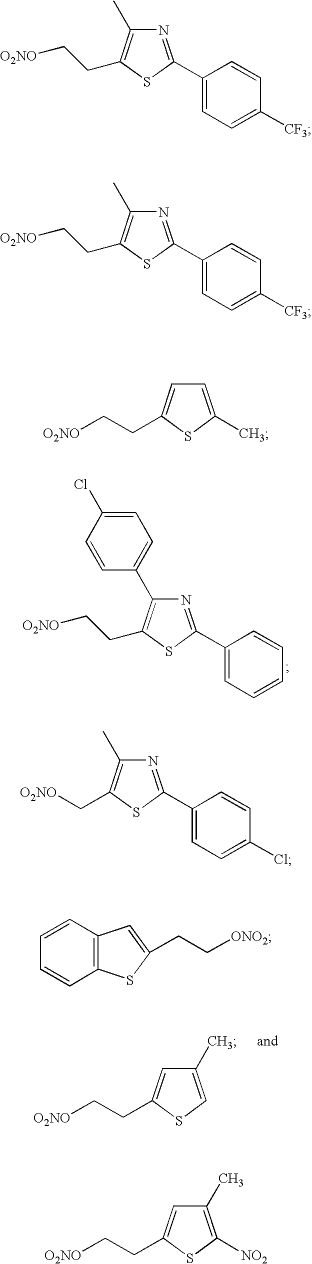

14. The compound of claim 8 selected from the following:

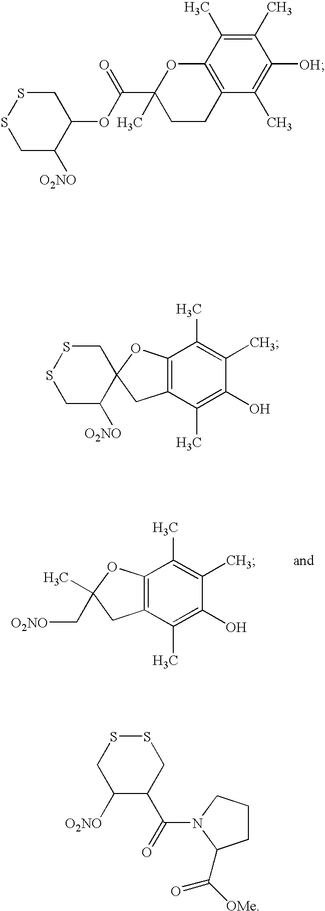

15. The compound of claim 8 selected from the following:

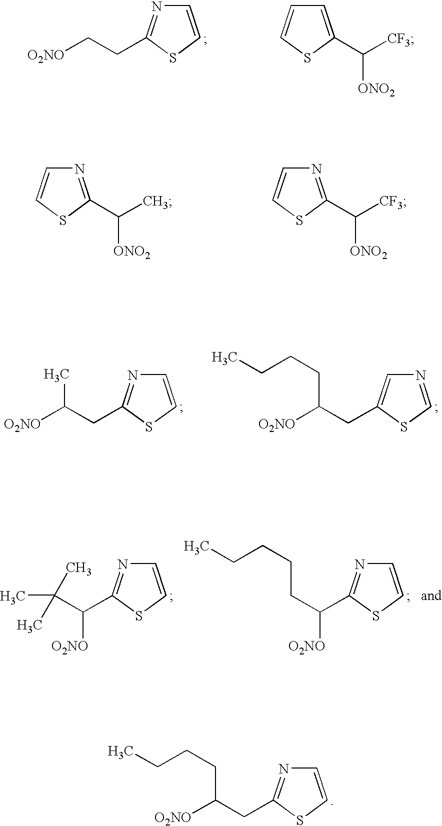

16. A nitrate ester selected from the following:

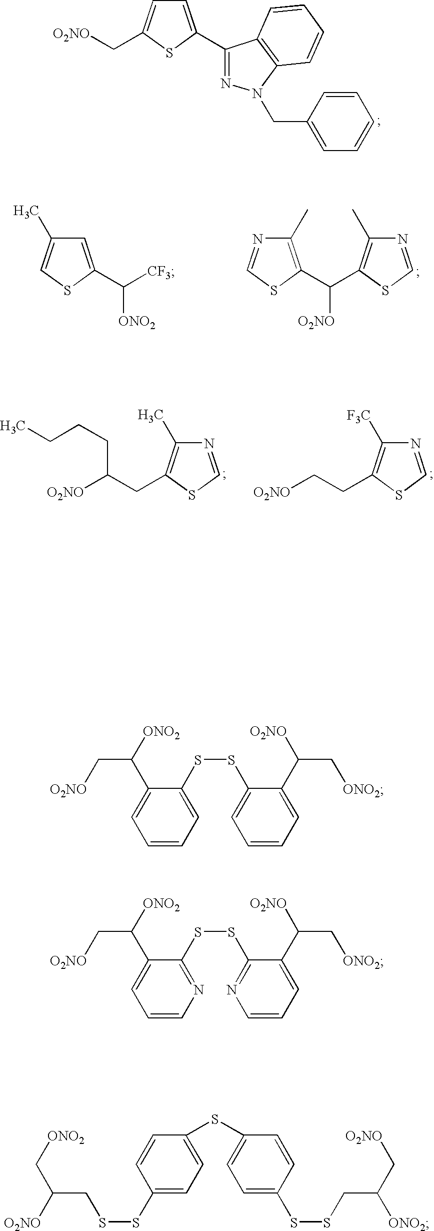

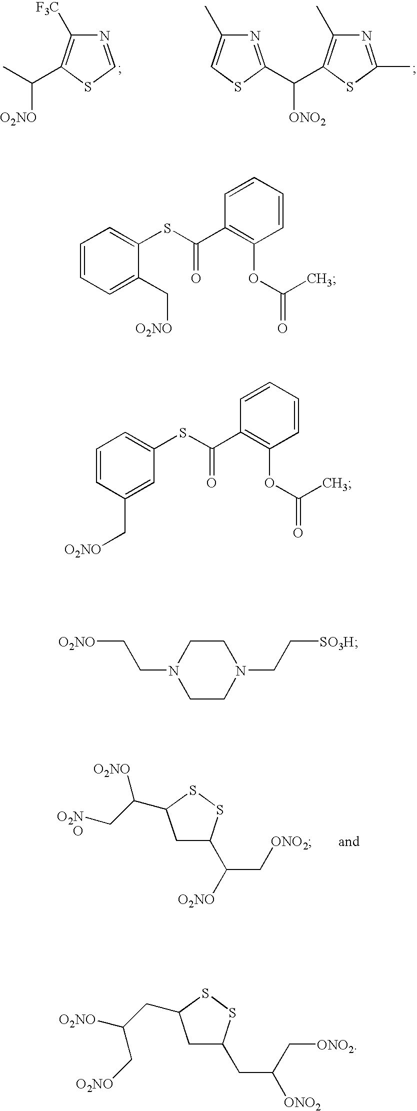

17. A composition comprising a pharmaceutically acceptable carrier and a compound having the general formula: 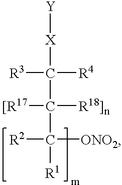
or a pharmaceutically acceptable salt thereof, containing from 1 to 3 nitrate groups and an S atom in proximity to a nitrate group,
wherein
each of m and n is, independently, an integer from 0 to 10;
R¹ is a hydrogen or A;
each of R², R⁵, and R¹⁸ is, independently, hydrogen or A;
each of R³, R⁴, and R¹⁷, is independently, a hydrogen, a nitrate group, or A;
ech of R⁶, R⁷, R⁸, R⁹, R¹¹, R¹², R¹³, R¹⁴, R¹⁵ and R¹⁶ is, independently, A, a hydrogen, a nitrate group, or a C₁-C₂₄ alkyl or acyl group, optionally containing 1-4 ONO₂ substituents or a C₁-C₆ linkage to R¹, R², R³, or R⁴ in cyclic derivatives;
each of R⁷ and R¹¹ is, independently, a substituted or unsubstituted C₁-C₈ alkyl or acyl group;
A is selected from:
a C₁-C₂₄ alkyl group, which optionally contains 1 to 4 O, S, NR⁶, and/or unsaturations in the chain, optionally bearing from 1 to 4 hydroxy, Cl, F, amino, unsubstituted or substituted aryl, unsubstituted or substituted heteroaryl, or unsubstituted or substituted heterocyclic groups, or 1-2 nitrate groups;
a C₃-C₂₄ alkyl group, containing 1-5 C═O, C═S, or C═NOR⁷ linkages, which optionally contains 1 to 4 O, S, NR⁶, and/or unsaturations in the carbon chain, optionally bearing from 1 to 4 hydroxy, nitrate, Cl, F, amino, unsubstituted or substituted aryl, unsubstituted or substituted heteroaryl, or unsubstituted or substituted heterocyclic groups;
a C₃-C₇ linkage to-any of R¹, R², R³, R⁴, or R¹⁷, forming an aliphatic ring, which optionally contains 1 to 2 O, S, NR⁶, and/or unsaturations in the linkage, optionally bearing from 1 to 6 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
a C₀-C₅ linkage to or between any of R¹, R³, R⁴, or R¹⁷, which optionally contains 1 to 2 O, S, NR⁶, and/or unsaturations in the linkage, bearing two or more substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
an unsubstituted C₀-C₅ linkage to or between any of R¹, R³, and R⁴, which optionally contains 1 to 2 non-adjacent O, S, NR⁶, and/or unsaturations in the linkage;
a C₁-C₅ linkage to or between any of R¹, R³, R⁴, and R¹⁷ containing 1 to 2 C═O, C═S, or C═NOR⁷ linkages, which optionally contains 1 to 2 O, S, NR⁶, and/or unsaturations in the linkage, optionally bearing from 1 to 4 substituents, independently selected from unsubstituted or substituted alkyl, unsubstituted or substituted aryl, unsubstituted or substituted C₁-C₄ alkaryl, unsubstituted or substituted heteroaryl, unsubstituted or substituted C₁-C₄ alkheteroaryl, unsubstituted or substituted heterocyclic, unsubstituted or substituted C₁-C₄ alkheterocyclic, hydroxy, nitrate, Cl, F, and amino groups;
a substituted or unsubstituted aryl group;
a substituted or unsubstituted heteroaryl group;
a substituted or unsubstituted heterocyclic group;
an amino, cyclic amino, diamino, triamino, alkylamino, dialkylamino, arylamino, diarylamino, or alkylarylamino group;
a hydroxy group;
an alkoxy group; and
a substituted or unsubstituted aryloxy group;
X is F, Br, NO₂, CH₂ CF₂, O, NH, NMe, NHOH, N₂H₃, N₂H₂R¹³, N₂HR¹³R¹⁴, N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)R⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷, or does not exist;
Y is F, Br, CH₃, CF₂H, CF₃, OH, NH₂, NHR⁶, NR⁶R⁷, NHOH, N₂H₃, N₂H₂R¹³N₂HR¹³R¹⁴, N₃, SC(═NH)N(R¹⁵)₂, SC(═NH)NHR¹⁵, SC(O)N(R¹⁵)₂, SC(O)NHR¹⁵, SH, SR⁷, SO₂M, S(O)R⁸, S(O)R⁵, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R⁵, C(O)(OR¹³), C(O)(SR¹³), C(S)R⁵, C(S)R¹², C(S)OR¹², or does not exist; and
M is H, Na⁺, K⁺, NH₄⁺, N⁺HₖR¹¹₍₄₋ₖ₎ where k is 0-3, or other pharmaceutically acceptable counterion;
and with the proviso that,
when m=0 and n=1;
each of R¹⁸ and R³ is, independently, a hydrogen, a nitrate group, or a C₁-C₄ alkyl chain, which may include one O, linking R¹⁸ and R³ together to form a pentosyl, a hexosyl, a cyclopentyl, or a cyclohexyl ring, said ring optionally bearing from 1-4 hydroxyl substituents;
each of R¹⁷ and R⁴ is, independently, a hydrogen, a nitrate group, a C₁-C₄ alkyl group, optionally bearing from 1-3 nitrate groups, or an acyl group (—C(O)R⁵);
each of R⁵, R⁶, R⁸, R⁹, R¹², R³, R¹⁴, R¹⁵, and R¹⁶ is, independently, a C₁-C₁₂ alkyl group, optionally bearing from 1-4 ONO₂ substituents, or a C₁-C₂ alkyl linkage to R¹⁸, R¹⁷, or R³;
X is F, Br, NO₂, CH₂ CF₂, O, NH, NMe, NHOH, N₂H₃, N₂H₂R¹³, N₂HR¹³R¹⁴, N₃, S, SC(═NH)N(R¹⁵)₂, SC(═NH)NHR¹⁵, SC(O)N(R¹⁵)₂, SC(O)NHR¹⁵, SH, SR⁵, SR⁷, S(O)R⁸, S(O)R⁵, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)(OR¹³), PO₂H, PO₂M, P(O)(OR¹⁴), P(O)(R¹³), SO, SO₂, C(O)(SR¹³), SR⁵, SR⁷; and
Y is not CN, N₂H₂R¹³, N₂HR¹³R¹⁴, N₃, SCN, SC(═NH)N(R¹⁵)₂, SC(═NH)NHR¹⁵, SC(O)N(R¹⁵)₂, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R⁵, C(O)R¹², C(O)(SR¹³), SR⁴, SR⁵, or SSR⁵, or Y does not exist.

18. A composition comprising a pharmaceutically acceptable carrier and a compound selected from:

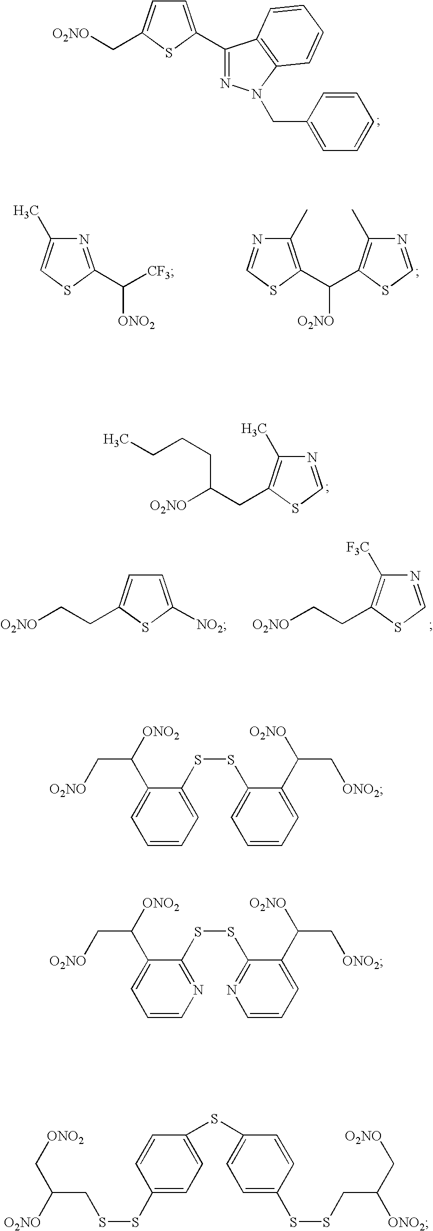

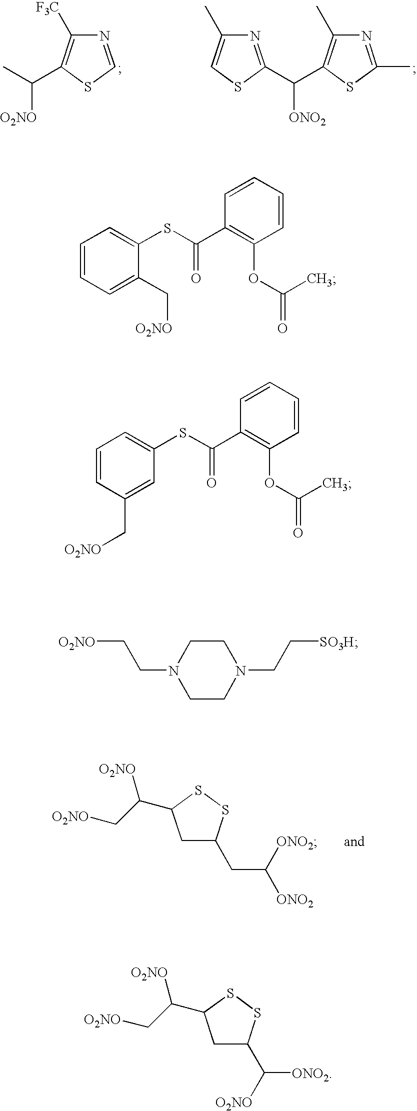

19. A method for preventing or mitigating tissue and/or cellular damage in a subject by modulating intercellular and/or intracellular free radical concentration in said subject, said method comprising administering to said subject an effective amount of a compound containing at least one aliphatic nitrate group and at least one sulfur atom in proximity to said nitrate.

20. The method of claim 19, wherein said compound has the formula: 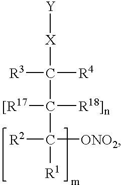
containing from 1 to 3 nitrate groups and an S atom in proximity to a nitrate group, wherein
m is an integer from 0 to 10;
n is an integer from 0 to 10;
each of R^{3,4,17} is, independently, hydrogen, a nitrate group, or A;
R¹ is hydrogen or A;
A is selected from:
a substituted or unsubstituted C₁-C₂₄ alkyl group, optionally containing 1 to 4 O, S, NR⁶, and/or unsaturations in the chain, optionally bearing from 1 to 4 hydroxy, nitrate, Cl, F, amino or unsubstituted or substituted aryl, or unsubstituted or substituted heterocyclic groups;
an unsubstituted or substituted cyclic moiety having from 3 to 7 carbon atoms in the ring, which optionally contains 1 to 2 O, S, NR⁶, and/or unsaturations in the ring, optionally bearing from 1 to 4 hydroxy, nitrate, Cl, F, amino or unsubstituted or substituted aryl, or unsubstituted or substituted heterocyclic groups;
an unsubstituted or substituted moiety constituting a linkage from 0 to 5 carbons, to or between any of R¹, R³, R¹⁷ and R⁴, which optionally contains 1 to 2 O, S, NR⁶, and/or unsaturations in the linkage, and optionally bearing from 1 to 4 hydroxy, nitrate, Cl, F, amino or unsubstituted or substituted aryl, or unsubstituted or substituted heterocyclic groups;
a substituted or unsubstituted C₁-C₂₄ alkyl group, containing 1-4 linkages selected from C═O, C═S, and C═NOH, which optionally contains O, S, NR⁶, and/or unsaturations in the chain, optionally bearing from 1 to 4 hydroxy, nitrate, Cl, F, amino or unsubstituted or substituted aryl, or unsubstituted or substituted heterocyclic groups;
a substituted or unsubstituted aryl group;
a substituted or unsubstituted heterocyclic group;
an amino group selected from alkylamino, dialkylamino, cyclic amino, cyclic diamino, cyclic triamino, arylamino, diarylamino, and alkyarylamino;
a hydroxy group;
an alkoxy group; and
a substituted or unsubstituted aryloxy group;
R², R⁵R¹⁸, are optionally hydrogen, A, or X—Y;
X is F, Br, Cl, NO₂, CH₂, CF₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)R⁵, S(O)₂R⁵, S(O)OR⁸, S(O)₂OR⁹, PO₂HM, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂H, PO₂M, P(O)(OR¹⁴), P(O)(R¹³), SO, SO₂, C(O)(SR¹³), SR⁵, SSR⁷ or SSR⁵, SS, or does not exist;
Y is F, Br, Cl, CH₃, CF₂H, CF₃, OH, NH₂, NHR⁶, NR⁶R⁷, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)R⁵, S(O)₂R⁵, S(O)₂OR⁹, PO₂HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R⁵, C(O)R¹², C(O)(OR¹³), C(O)(SR¹³), SR⁵, SSR⁷ or SSR⁵, C(S)R⁵, C(S)R¹², C(S)OR¹², or does not exist;
each of R⁶, R⁷, R⁸, R⁹, R¹¹, R¹², R¹³, R¹⁴, R¹⁵, and R¹⁶ is, independently, a C₁-C₂₄ alkyl group, optionally containing 1-4 ONO₂ substituents, a C₁-C₂₄ acyl group, optionally containing 1-4 ONO₂ substituents, a C₁-C₆ ring-forming connection to any of R¹-R⁴, a hydrogen, a nitrate group, or A; and
M is H, Na⁺, K⁺, NH₄⁺, N⁺HₖR¹¹₍₄₋ₖ₎ where k is 0-3, or other pharmaceutically acceptable counterion;
and with the proviso that,
when m=0; n=1;
each of R¹⁸ and R³ is, individually, H, a nitrate group, or a C₁-C₄ alkyl group, which may include one O, linking R¹⁸ and R³ to form pentosyl, hexosyl, cyclopentyl, or cyclohexyl rings, which optionally bears hydroxyl substituents;
each of R¹⁷ and R⁴ is, individually, H, a nitrate group, a C₁-C₄ alkyl group, optionally bearing from 1-3 nitrate groups, or —C(O)R⁵;
each of R⁵, R⁶, R⁸, R⁹, R¹², R¹³, R¹⁴, R¹⁵, R¹⁶ is, individually, a C₁-C₁₂ alkyl group, optionally containing 1-4 ONO₂ substituents or a C₁-C₂ ring-forming connections to R¹⁸, R¹⁷, or R³;
each of R⁷ and R¹¹ is, independently, a C₁-C₈ alkyl group or a C₁-C₈ acyl group;
M is H, Na⁺, K⁺, NH₄⁺, N⁺HₖR¹¹₍₄₋ₖ₎ where k is 0-3;
X is CH₂, O, NH, NMe, CN, NHOH, N₂H₃, N₂H₂R¹³, N₂HR¹³R¹⁴, N₃, S, SCN, SC(═NH)N(R¹⁵)₂, SC(═NH)NHR¹⁵, SC(O)N(R¹⁵)₂, SC(O)NHR¹⁵, SO₃M, SH, SR⁷, SO₂M, S(O)R⁸, S(O)₂R⁹, S(O)OR⁸, S(O)₂OR⁹, PO₃HM, PO₃M₂, P(O)(OR¹⁵)(OR¹⁶), P(O)(OR¹⁶)(OM), P(O)(R¹⁵)(OR⁸), P(O)(OM)R¹⁵, CO₂M, CO₂H, CO₂R¹¹, C(O), C(O)R¹², C(O)(OR¹³), PO₂M, P(O)(OR¹⁴), P(O)(R¹³), SO, SO₂, C(O)(SR¹³), SR⁵, or SSR⁴; and
Y is not CN, N₂H₂R¹³, N₂HR¹³R¹⁴, N₃, SCN, SC(═NH)N(R¹⁵)₂, SC(═NH)NHR¹⁵, SC(O)NHR¹⁵, SO₃M, SH, SO₂M, PO₃M₂, PO₃HM, P(O)(OR¹⁵)(OR¹⁶), P(O)(OR¹⁶)(OM), P(O)(OM)R¹⁵, CO₂M, CO₂H, CO₂R⁵, C(O)R¹², C(O)(SR¹³), SR⁴, SR⁵, or SSR⁵, or Y does not exist.

21. The method of claim 19, wherein said nitrate contains at least 2 nitrate groups.

22. The method of claim 19, wherein said nitrate is beta or gamma to said sulfur atom.

23. The method of claim 19, wherein said compound is selected from: 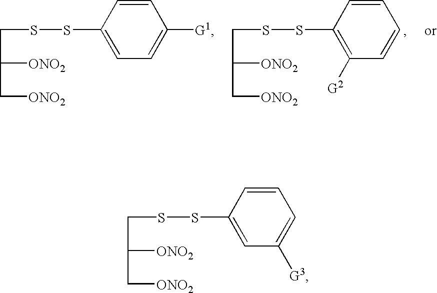
wherein G¹ is Me, OMe, Cl, NO₂, Br, or H; G² is CO₂Et, CO₂H, CO₂Me, CONH₂, or CO(CH₂)₂NEt₂, and G³ is Cl, OMe, or CONH₂;

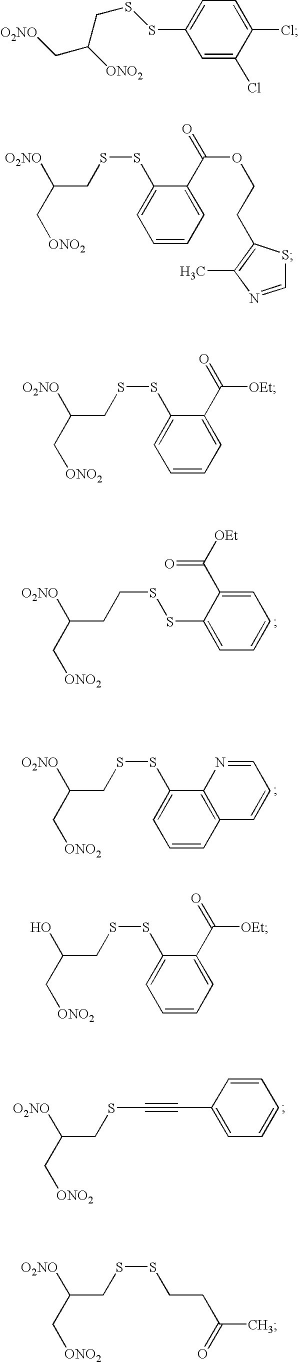

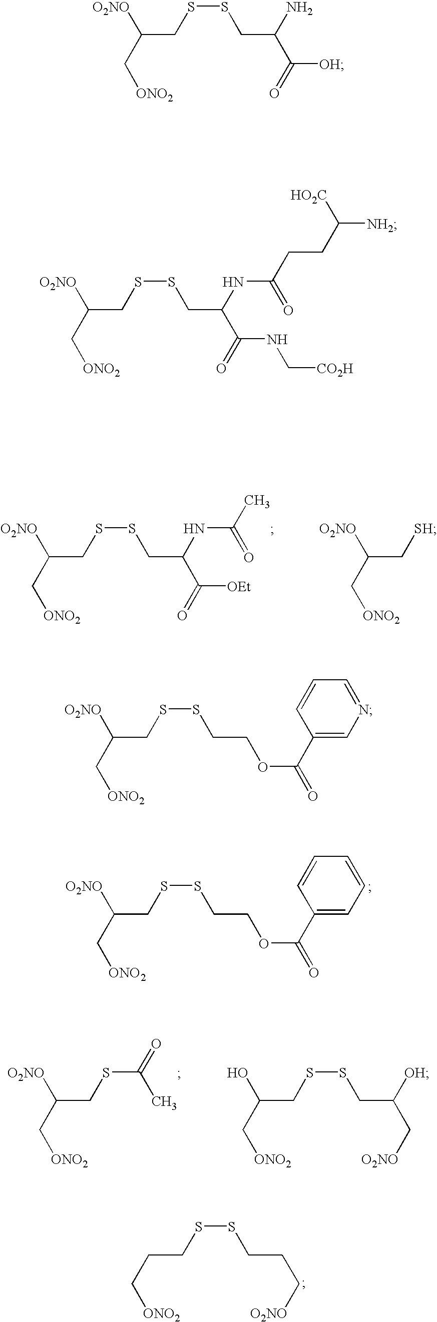

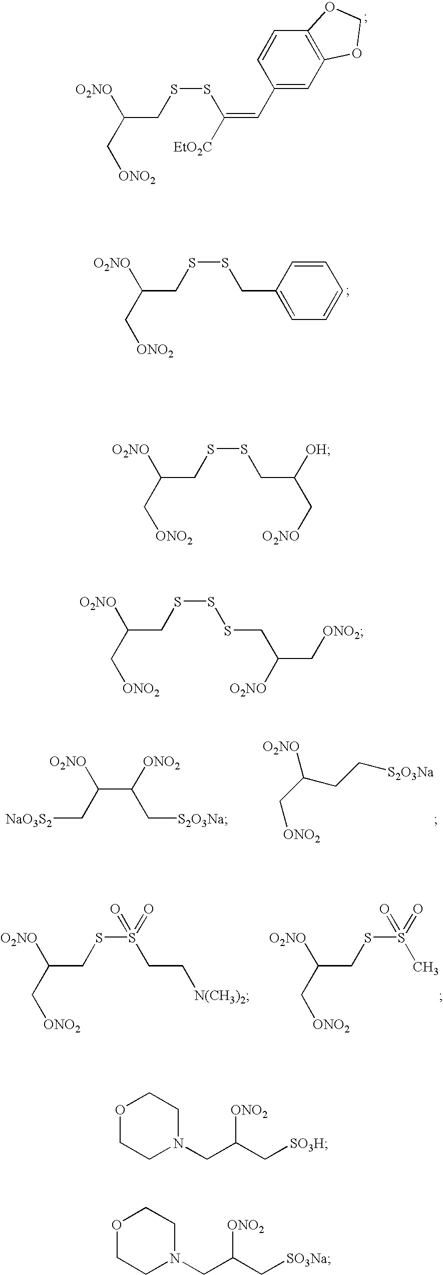

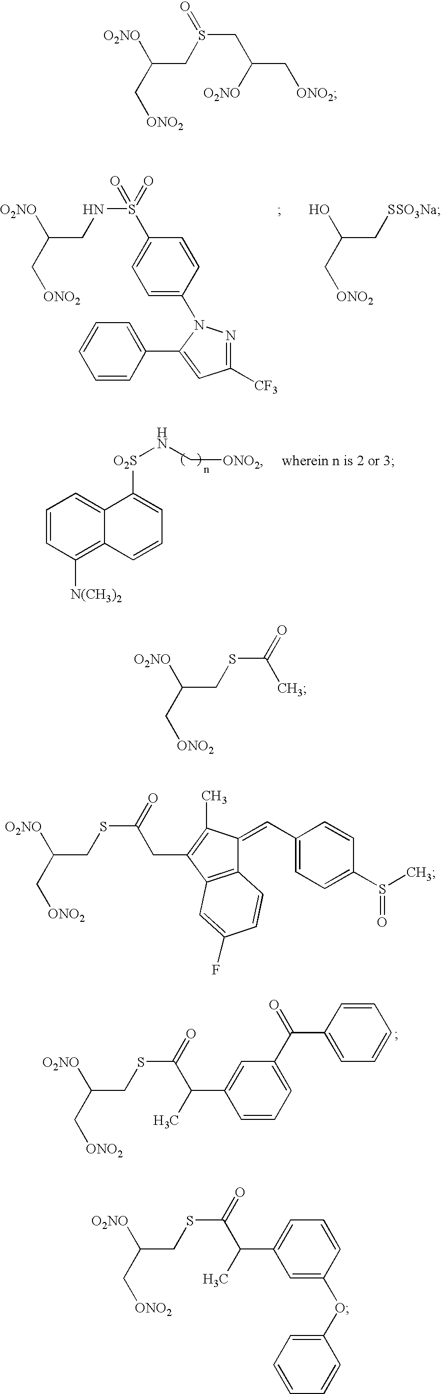

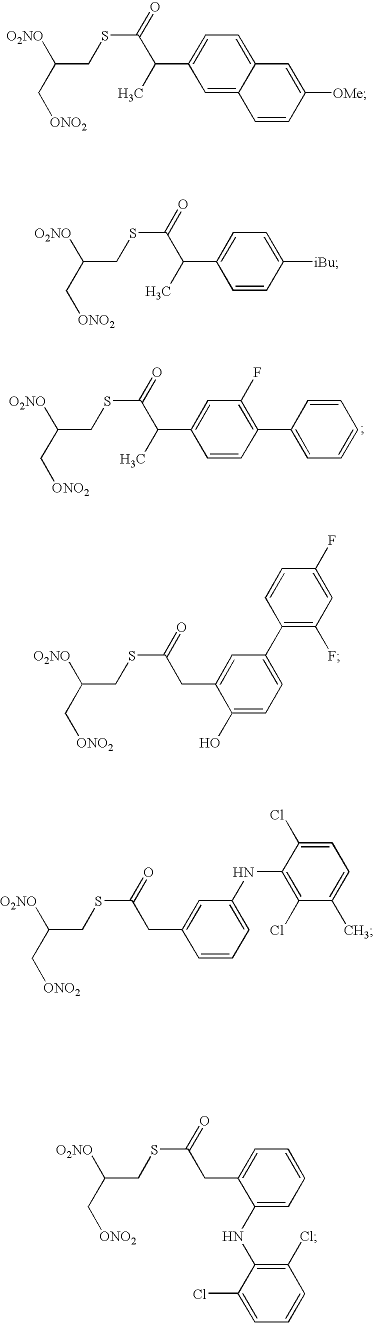

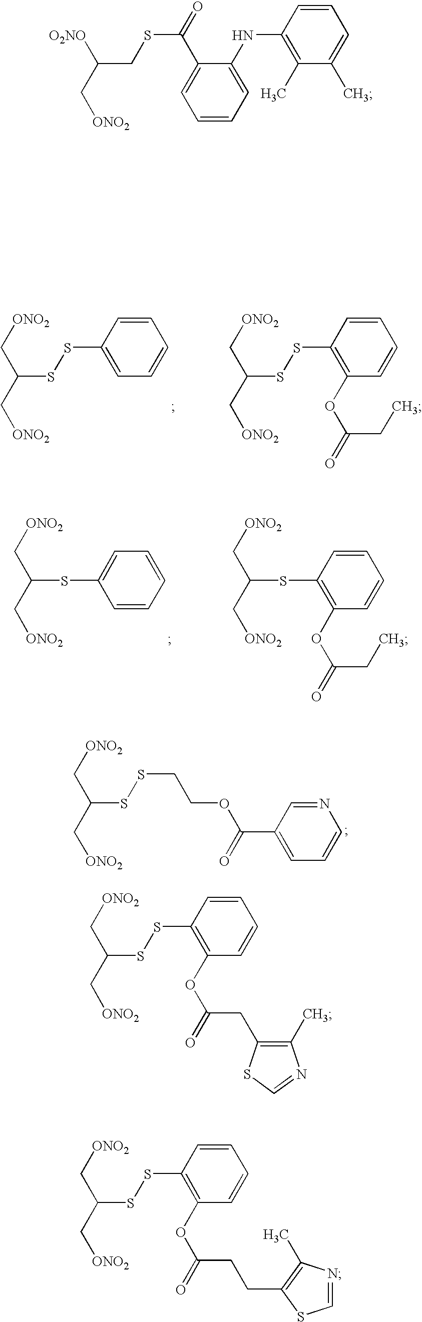

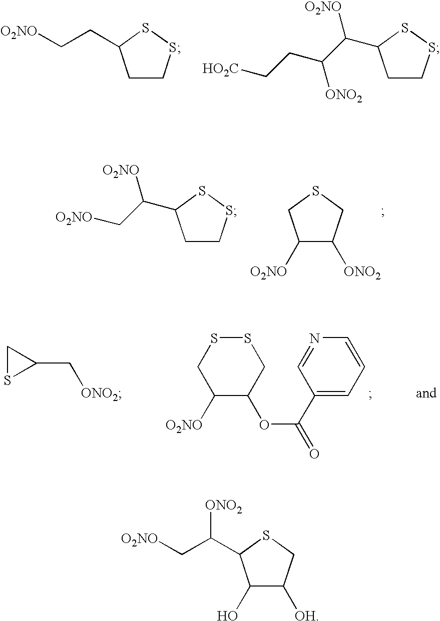

24. The method of claim 19, wherein said compound is selected from: 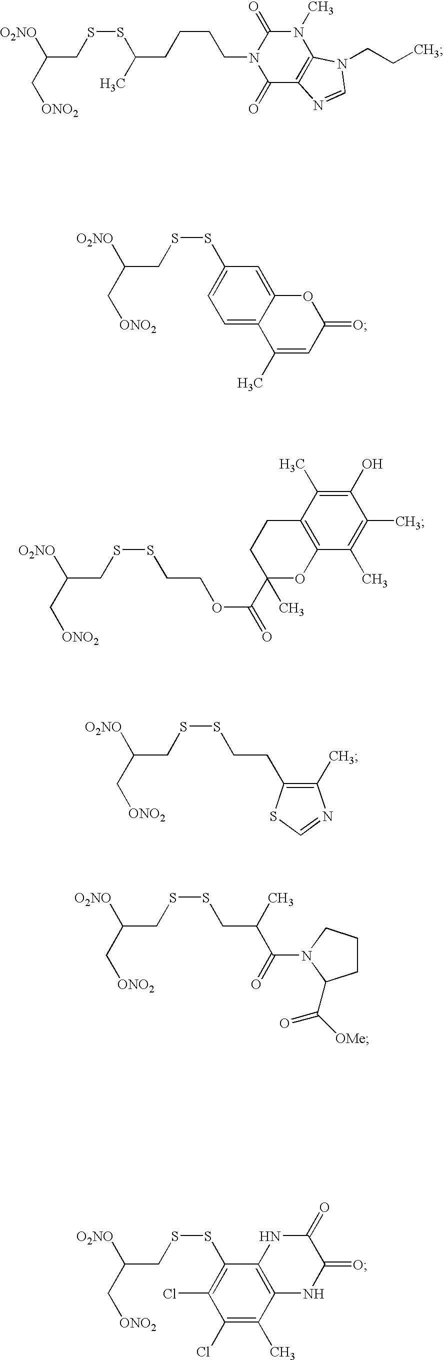 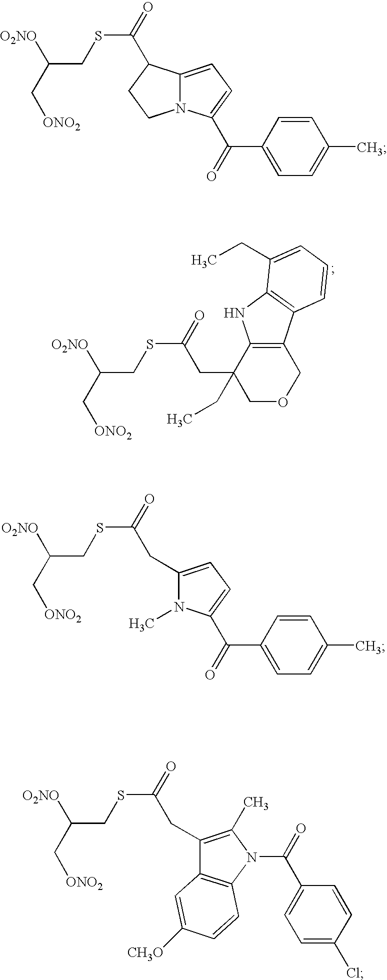 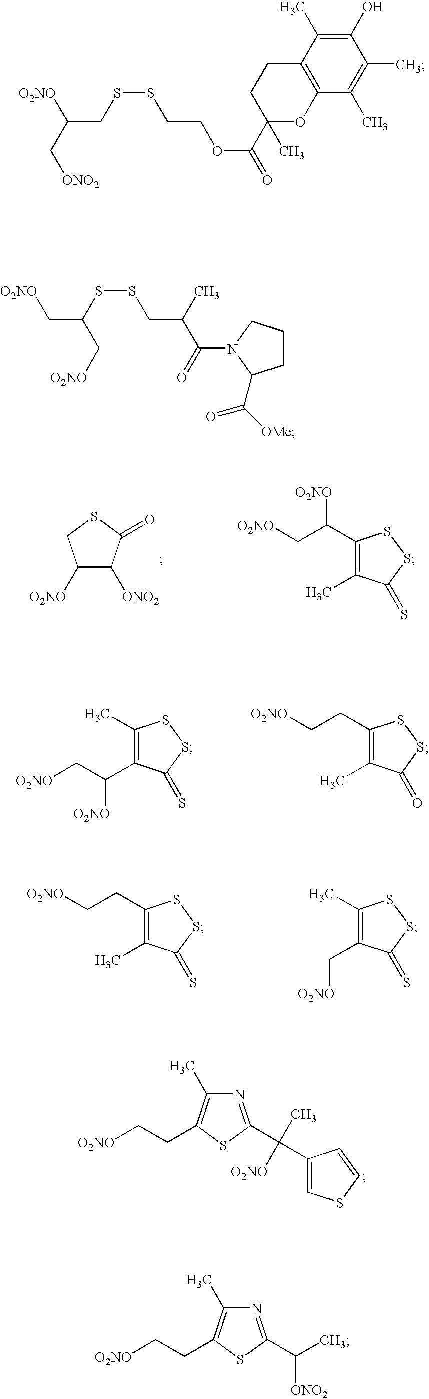 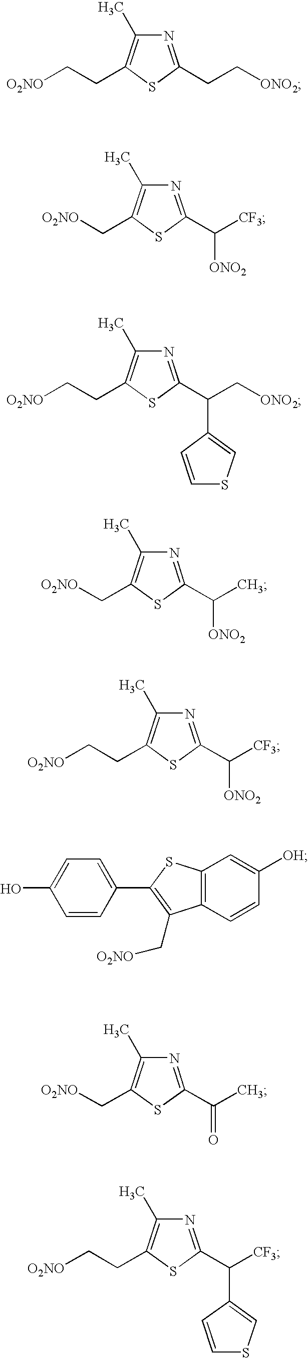 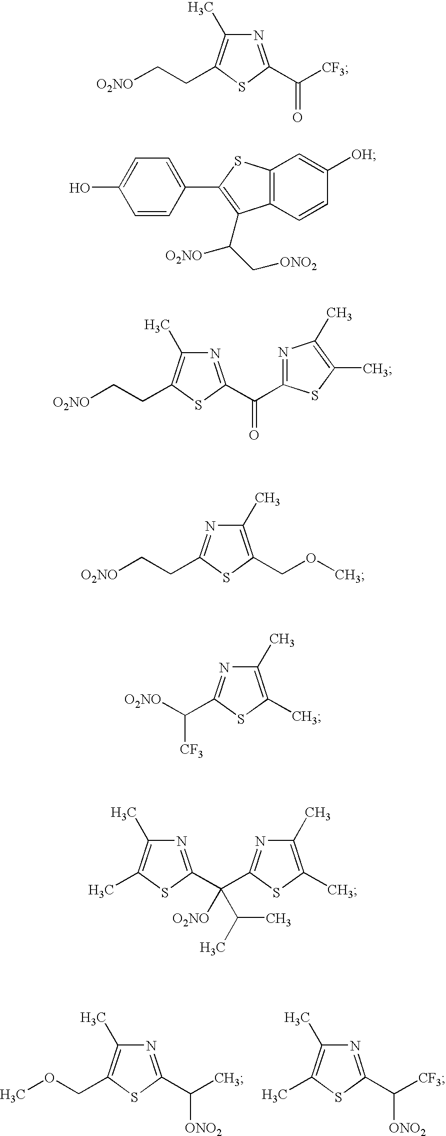 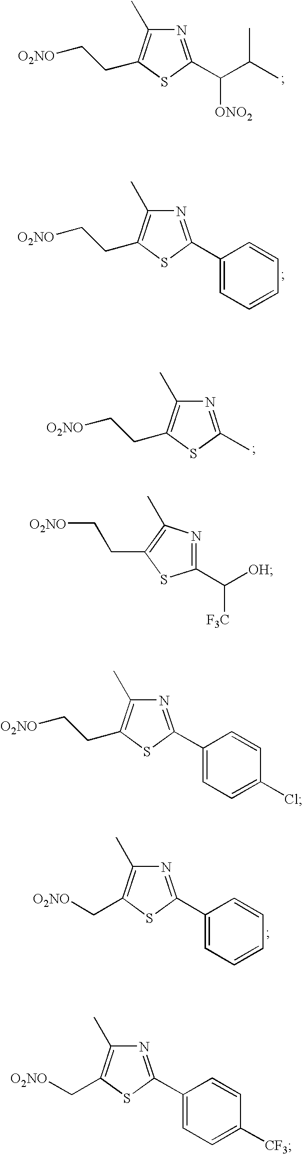 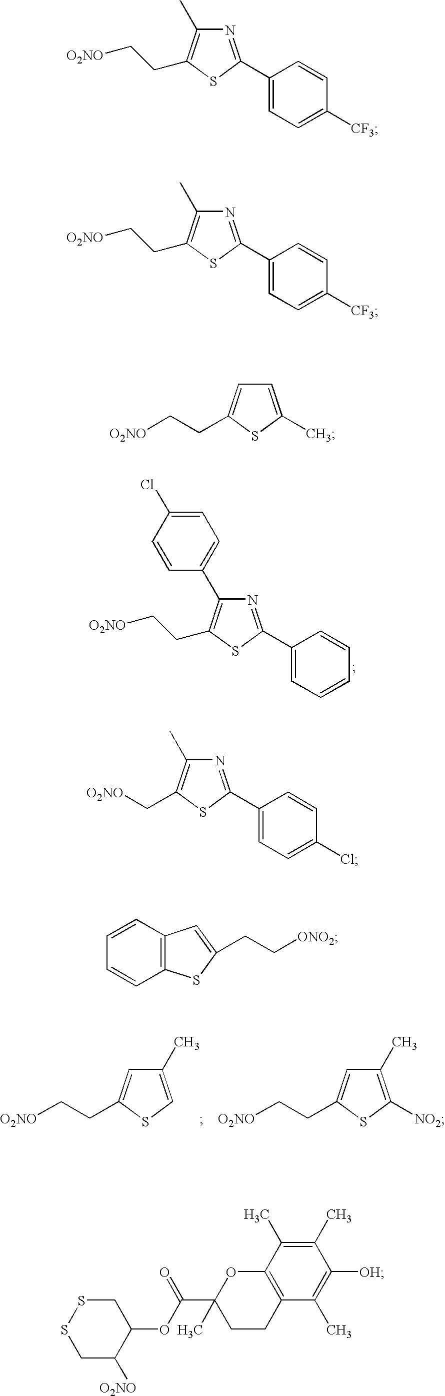 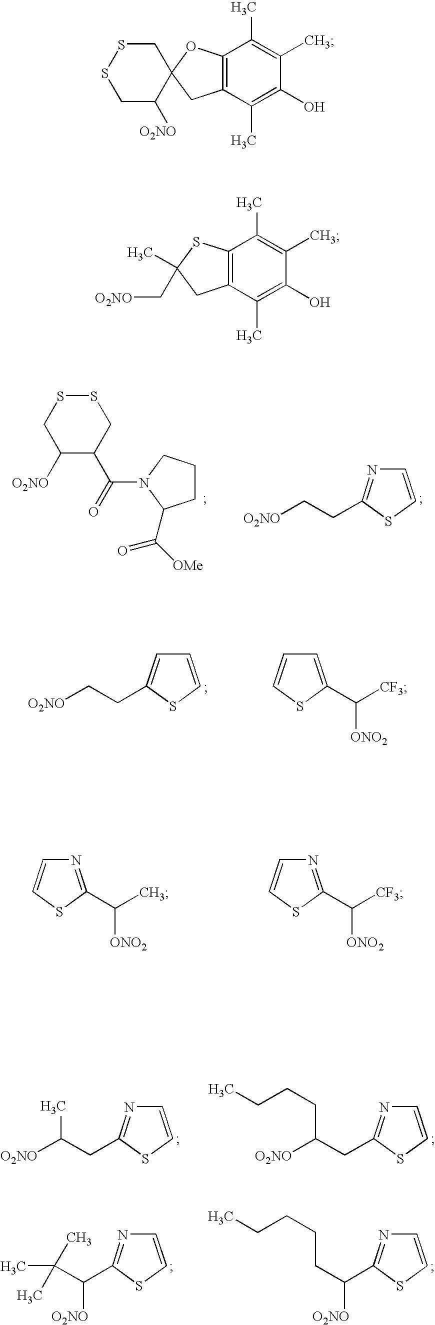 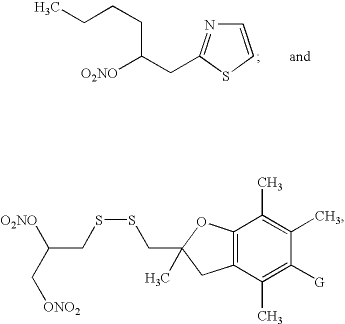
wherein G is Br, OH, NH₂, or NHCHO.

25. The method of claim 19, wherein said compound is selected from:

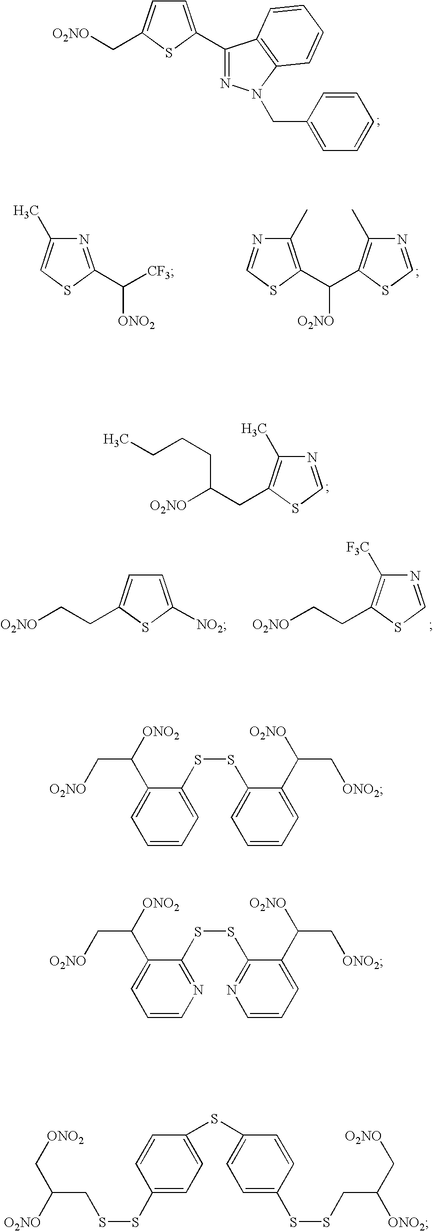

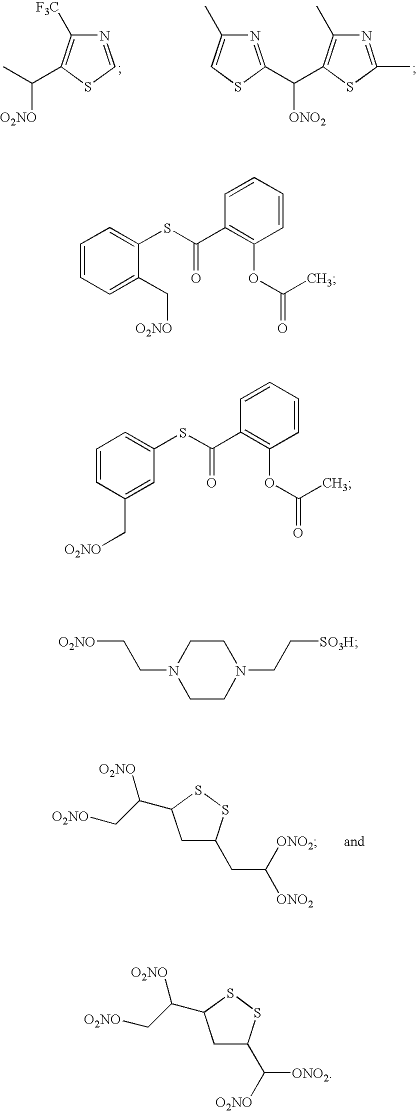

26. The method of claim 19, wherein said compound is selected from:

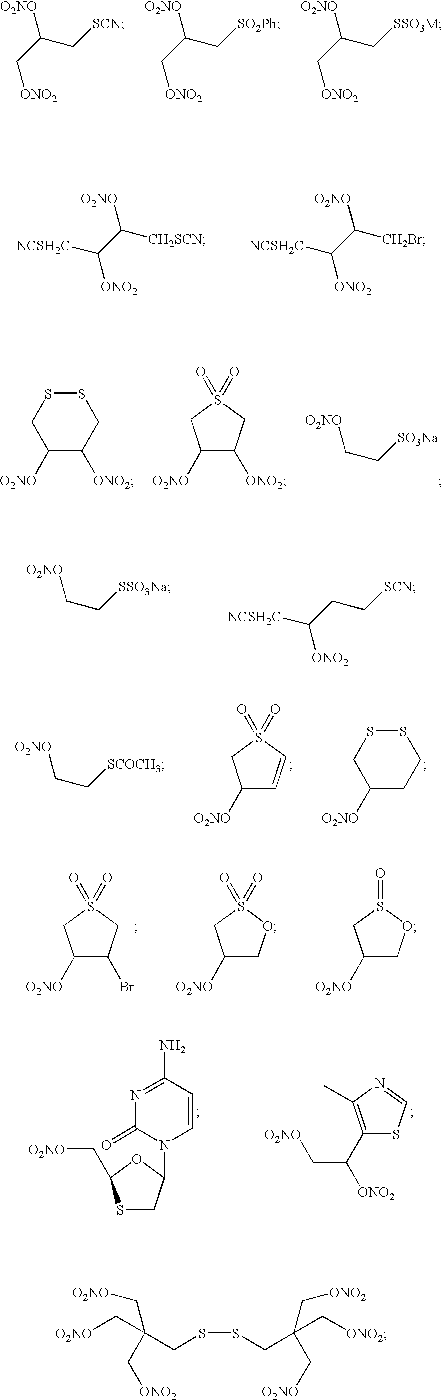

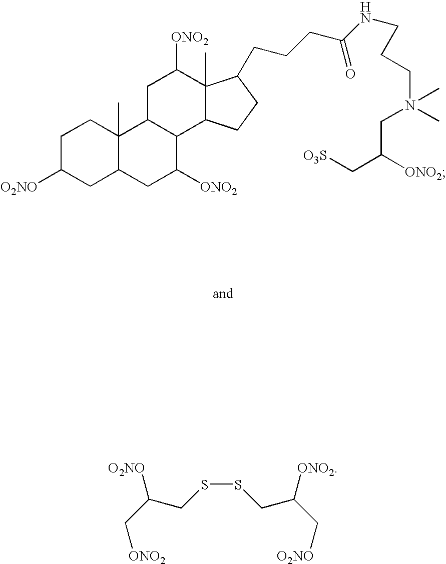

27. The method of claim 19, wherein said compound is:

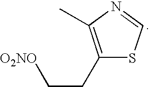

28. The method of claim 19, wherein said tissue and/or cellular damage is associated with aging, septic shock, ischemia/reperfusion injury, ulcers, gastritis, ulcerative colitis, Crohn's disease, diabetes, rheumatoid arthritis, asthma, cirrhosis of the liver, allograft rejection, encephalomyelitis, meningitis, pancreatitis, peritonitis, vasculitis, lymphocytic choriomeningitis, glomerulonephritis, uveitis, glaucoma, blepharitis, chalazion, allergic eye disease, corneal ulcer, keratitis, cataracts, age-related macular degeneration, optic neuritis, ileitis, hemorrhagic shock, anaphylactic shock, bacterial infection, viral infection, fungal infection, parasitic infection, hemodialysis, chronic fatigue syndrome, stroke, toxic shock syndrome, adult respiratory distress syndrome, cachexia, myocarditis, eczema, psoriasis, dermatitis, urticaria, cerebral ischemia, systemic lupus erythematosis, chronic neurodegenerative disease, priapism, cystic fibrosis, schizophrenia, depression, premenstrual syndrome, anxiety, addiction, migraine, gastrointestinal motility disorders, obesity; hyperphagia, hematologic cancers, myelofibrosis, graft-versus-host disease, CNS trauma, hepatitis, renal failure, chronic hepatitis C, drug-induced lung injury, fertility enhancement, bacterial translocation, circulatory shock, traumatic shock, myocardial infarction, or vascular aneurysm.

29. The method of claim 19, wherein said tissue and/or cellular damage is associated with Parkinson's disease; Alzheimer's disease; Huntington's disease; multiple sclerosis; amylotrophic lateral sclerosis; AIDS-induced dementia; epilepsy; alcoholism; alcohol withdrawal; drug-induced seizures; viral/bacterial/fever-induced seizures; trauma to the head; hypoglycemia; hypoxia due to myocardial infarct; cerebral vascular occlusion; cerebral vascular hemorrhage; hemorrhage; or environmental excitotoxins of plant, animal, or marine origin

30. The method of claim 19, wherein said tissue and/or cellular damage is associated with cytokine therapy, wherein said compound is administered to said subject before, during, and/or after the administration of said cytokine to said subject.
